(19) United States
(12) Patent Application Publication
(10) Pub. No.: US 2026/0284042 A1
(43) Pub. Date: Sep. 24, 2026

(54) DOSING REGIMEN

(71) Applicant: ViiV Healthcare UK (No.3) Limited, Stevenage (GB)

(72) Inventors: Kelong HAN, Collegeville, PA (US); Jon COLLINS, Durham, NC (US); Alex RINEHART, Durham, NC (US)

(73) Assignee: ViiV Healthcare UK (No.3) Limited, Stevenage (GB)

(21) Appl. No.: 19/464,746

(22) Filed: Jan. 30, 2026

Related U.S. Application Data
(63) Continuation of application No. PCT/IB2025/052124, filed on Feb. 27, 2025.
(60) Provisional application No. 63/560,027, filed on Mar. 1, 2024.

(51) Int. Cl. A61K 31/4985; A61K 47/10; A61K 47/26; A61K 47/38

(52) U.S. Cl. CPC A61K 31/4985; A61K 47/10; A61K 47/26; A61K 47/38

(57) ABSTRACT

Invented are methods for preventing or treating HIV in a human, comprising administering to said human a pharmaceutical composition comprising cabotegravir once every 15 to 24 weeks.

### FIELD OF THE INVENTION

The invention relates to Human Immunodeficiency Virus (HIV) treatment or prevention. In particular, the invention relates to a dosing regimen for long-acting treatment or prevention of HIV.

### BACKGROUND TO THE INVENTION

Patients with HIV infection commonly undergo complex treatment regimens which involve taking multiple pills at regular intervals each day. Patient non-compliance is a known problem accompanying these complex HIV treatment regimens and can lead to the emergence of multiple drug resistant strains of HIV.

The application of long-acting parenteral pharmaceuticals has been established in clinical practice for decades, notably in the areas of contraception, anti-psychotics, and opiate addiction. More recently, long-acting parenteral pharmaceuticals have been proposed as a way of overcoming the non-compliance problem with HIV treatment regimens. Long-acting injectable formulations, some of which are approved and marketed, such as CABENUVA® and APRETUDE®, have demonstrated prolonged exposures (≥30 days) following injection, enabling dosing at once-monthly and bimonthly intervals.

However, patients and people at risk of HIV infection would benefit from dosing still less frequently. This would help overcome problems with patient compliance and reduce burden on healthcare providers administering long-acting products.

Previous attempts to create high concentration formulations of cabotegravir suitable for dosing regimens with longer intervals between dosing has been challenging for a number of reasons. Formulations need to be suitable for long-acting injections and stable when stored whilst also giving a similar pharmacokinetic and safety profile to the currently approved regimen i.e. achieve at least equal efficacy to the currently approved cabotegravir regimen. Further, any dosing regimen would need to have minimal injection site reactions (ISRs) i.e. pain, erythema and swelling.

Formulations and/or dosages containing high amounts of cabotegravir do not always have the required properties that make them suitable for longer acting dosing to humans, for example, the marketed cabotegravir 200 mg/mL formulation has been shown to lack physical stability when concentration is increased up to 400 mg/mL. Further, the marketed cabotegravir 200 mg/mL formulation was administered to humans in a phase 1 trial in doses up to 3200 mg/16 mL subcutaneously in combination with rHuPH20, a recombinant human hyaluronidase that increases subcutaneous drug absorption and dispersion. Whilst the results obtained from this regimen indicated potentially increased bioavailability, all study participants experienced ISRs including one participant who experienced a drug related adverse event (AE) of injection site erythema with necrosis which led to discontinuation of this approach.

Achieving a dosing regimen of cabotegravir with longer intervals between dosing which has a favourable safety and pharmacokinetic profile, whilst maintaining patient experience and minimising injection site reactions, is desirable.

### SUMMARY OF THE INVENTION

According to a first aspect of the invention, there is provided a method of preventing HIV in a human, comprising administering to said human a pharmaceutical composition comprising cabotegravir once every 15 to 24 weeks.

According to a second aspect of the invention, there is provided a method of treating HIV in a human in need thereof, comprising administering to said human a pharmaceutical composition comprising cabotegravir once every 15 to 24 weeks.

According to a third aspect of the invention, there is provided a pharmaceutical composition for use in the treatment of HIV, wherein the pharmaceutical composition comprises cabotegravir and the treatment comprises administering the pharmaceutical composition to a human once every 15 to 24 weeks.

According to a fourth aspect of the invention, there is provided a pharmaceutical composition for use in the prevention of HIV, wherein the pharmaceutical composition comprises cabotegravir and the prevention comprises said prevention comprising administering the pharmaceutical composition to a human once every 15 to 24 weeks.

According to a fifth aspect of the invention, there is provided the use of a pharmaceutical composition in the manufacture of a medicament for treating an HIV infection, wherein the pharmaceutical composition comprises cabotegravir.

According to a sixth aspect of the invention, there is provided the use of a pharmaceutical composition in the manufacture of a medicament for preventing an HIV infection, wherein the pharmaceutical composition comprises cabotegravir.

The methods of treatment and prevention and pharmaceutical compositions for use in treatment and prevention of the present invention are advantageous in a number of respects. The inventors have found that dosing regimens of pharmaceutical compositions comprising cabotegravir of the present invention have favourable safety and pharmacokinetic profiles allowing them to be dosed at longer intervals than the currently approved regimens whilst still maintaining favourable patient experience, namely low or tolerable injection site reactions (ISRs). Longer dosing intervals will contribute to improved patient compliance as well as reduced administration burden on health care providers.

### BRIEF DESCRIPTION OF THE DRAWINGS

FIG. 1 shows a simulation of a median plasma concentration/time plot of a human assigned male at birth who has had an intramuscular loading dose of 3200 mg of cabotegravir (CAB) (a 533 mg/mL formulation of cabotegravir in 3 mL twice-formulation as described herein in cohort 5, example 9) and, 1 month later a first maintenance dose of 1600 mg of cabotegravir and then 1600 mg of cabotegravir every 4 months (a 533 mg/ml formulation of cabotegravir in 3 mL as described herein in cohort 5, example 9). This has been overlaid over the plot of a median plasma concentration over time of a human assigned male at birth who has been administered the approved Cabotegravir 200 mg/mL formulation Q2M (every 2 months) (APRETUDE®-labelled CAB200 in the figure). The black line in the middle of the shaded grey area is the simulated median of the dosing regimen (name CAB-ULA in the figure). The black line in the middle of the grey shaded area is higher than the black dashed line (the median plasma concentration of CAB200). The lower boundary of the grey band (simulated 10th percentile of CAB-ULA) is higher than the lowest dashed line (simulated 10th percentile of CAB200). This thus shows that this regimen maintains CAB plasma concentrations higher than the approved Q2M regimen of CAB200 IM gluteal injections.

FIG. 2 shows a simulation of a median plasma concentration/time plot of a human assigned female at birth who has had an intramuscular loading dose of 3200 mg of cabotegravir (a 533 mg/mL formulation of cabotegravir in 3 mL twice-formulation as described herein in cohort 5, example 9) and, 1 month later a first maintenance dose of 1600 mg of cabotegravir and then 1600 mg of cabotegravir every 4 months (a 533 mg/mL formulation of cabotegravir in 3 mL as described herein in cohort 5, example 9). This has been overlaid over the plot of a median plasma concentration over time of a human assigned female at birth who has been administered the approved Cabotegravir 200 mg/mL formulation Q2M (every 2 months) (APRETUDE®-labelled CAB200 in the figure). The black line in the middle of the shaded grey area is the simulated median of the dosing regimen (name CAB-ULA in the figure). The black line in the middle of the grey shaded area is higher than the black dashed line (the median of CAB200). The lower boundary of the grey band (simulated 10th percentile of CAB-ULA) is higher than the lowest dashed line (simulated 10th percentile of CAB200). This thus shows that this regimen maintains CAB plasma concentrations higher than the approved Q2M regimen of CAB200 IM gluteal injections.

FIG. 3 shows a simulation of a median plasma concentration/time plot of a human assigned male at birth who has had an intramuscular loading dose of 2132 mg cabotegravir (a 533 mg/mL formulation of cabotegravir in 4 mL) and, 1 month later a first maintenance dose of 799.5 mg of cabotegravir and then 799.5 mg of cabotegravir every 4 months (a 533 mg/mL formulation of cabotegravir in 1.5 mL). This has been overlaid over the plot of a median plasma concentration over time of a human assigned male at birth who has been administered approved Cabotegravir 200 mg/mL formulation Q2M. The dotted black line is the median plasma concentration of the approved regimen (APRETUDE®). The grey shaded area shows 10th to 90th percentile plasma concentrations of the simulated regimen. The solid black line is the median of the simulated dosing regimen.

FIG. 4 shows a simulation of a median plasma concentration/time plot of a human assigned female at birth who has had an intramuscular loading dose of 2132 mg cabotegravir (a 533 mg/mL formulation of cabotegravir in 4 mL) and, 1 month later a first maintenance dose of 799.5 mg of cabotegravir and then 799.5 mg of cabotegravir every 4 months (a 533 mg/mL formulation of cabotegravir in 1.5 mL). This has been overlaid over the plot of a median plasma concentration over time of a human assigned female at birth who has been administered approved Cabotegravir 200 mg/ml formulation Q2M. The dotted black line is the median plasma concentration of the approved regimen (APRETUDE®). The grey shaded area shows 10th to 90th percentile plasma concentrations of the simulated regimen. The solid black line is the median of the simulated dosing regimen.

FIG. 5 shows a simulation of a median plasma concentration/time plot of a human assigned male at birth who has had an intramuscular loading dose of cabotegravir (a 400 mg/ml formulation of cabotegravir in 3 mL twice) and, 1 month later a first maintenance dose of 1200 mg of cabotegravir and then 1200 mg of cabotegravir every 4 months (a 400 mg/mL formulation of cabotegravir in 3 mL, see cohort 3, example 9 herein). This has been overlaid over the plot of a median plasma concentration over time of a human assigned male at birth who has been administered approved Cabotegravir 200 mg/mL formulation Q2M. The dotted black line in the middle of the shaded grey area is the median plasma concentration of the approved regimen (APRETUDE®). The grey shaded area is the interval of the approved regimen. The solid black line is the median of the simulated dosing regimen and the dotted black lines above and below it are the upper and lower boundaries of the predicted interval of the simulated dosing regimen.

FIG. 6 shows a simulation of a median plasma concentration/time plot of a human assigned female at birth who has had an intramuscular loading dose of cabotegravir (a 400 mg/mL formulation of cabotegravir in 3 mL twice) and, 1 month later a first maintenance dose of 1200 mg of cabotegravir and then 1200 mg of cabotegravir every 4 months (a 400 mg/mL formulation of cabotegravir in 3 mL, see cohort 3, example 9 herein). This has been overlaid over the plot of a median plasma concentration over time of a human assigned female at birth who has been administered approved Cabotegravir 200 mg/mL formulation Q2M. The dotted black line in the middle of the shaded grey area is the median plasma concentration of the approved regimen (APRETUDE®). The grey shaded area is the interval of the approved regimen. The solid black line is the median of the simulated dosing regimen and the dotted black lines above and below it are the upper and lower boundaries of the predicted interval of the simulated dosing regimen.

FIG. 7 shows the same simulation as shown in FIG. 1 but overlaid over a dashed line showing 1.05 μg/mL rather than a simulation of the approved regimen. The lower boundary of the grey band (simulated 10th percentile of the dosing regimen shown), labelled as CAB-ULA in the figure, is higher than the horizontal dashed line i.e. the benchmark for efficacy in people assigned male at birth.

FIG. 8 shows the same simulation as shown in FIG. 2 but overlaid over a dashed line showing 1.39 μg/mL rather than a simulation of the approved regimen. The lower boundary of the grey band (simulated 10th percentile of the dosing regimen shown), labelled as CAB-ULA in the figure, is higher than the horizontal dashed line i.e. the benchmark for efficacy for people assigned female at birth.

FIG. 9 shows a simulation of a median plasma concentration/time plot of a human assigned male at birth who has had an intramuscular loading dose of 2132 mg of cabotegravir (533 mg/mL of cabotegravir in 4 mL) and, 1 month later a first maintenance dose of 1066 mg of cabotegravir and then 1066 mg of cabotegravir every 4 months (533 mg/ml of cabotegravir in 2 mL). This has been overlaid over the plot of a median plasma concentration over time of a human assigned male at birth who has been administered approved Cabotegravir 200 mg/mL formulation Q2M. The dotted black is the median plasma concentration of the approved regimen (APRETUDE®). The solid black line is the median of the simulated dosing regimen (labelled Cab-ULA) and the shaded grey area around is it is the predicted interval of the simulated dosing regimen from the 10th to 90th percentile.

FIG. 10 shows a simulation of a median plasma concentration/time plot of a human assigned female at birth who has had an intramuscular loading dose of 2132 mg of cabotegravir (533 mg/ml of cabotegravir in 4 mL) and, 1 month later a first maintenance dose of 1066 mg of cabotegravir and then 1066 mg of cabotegravir every 4 months (533 mg/ml of cabotegravir in 2 mL). This has been overlaid over the plot of a median plasma concentration over time of a human assigned female at birth who has been administered approved Cabotegravir 200 mg/mL formulation Q2M. The dotted black is the median plasma concentration of the approved regimen (APRETUDE®). The solid black line is the median of the simulated dosing regimen (labelled Cab-ULA) and the shaded grey area around is it is the predicted interval of the simulated dosing regimen from the 10th to 90th percentile.

FIGS. 11 and 12 show a simulation from a second set of simulations based on CAB ULA PopPK model, wherein a 1600 mg CAB ULA (3 mL of 533 mg/mL cabotegravir) IM gluteal injection (loading dose) is followed by a maintenance regimen of 1600 mg CAB ULA (3 mL of 533 mg/mL cabotegravir) in both people assigned male at birth (FIG. 11) and people assigned female at birth (FIG. 12). Dashed lines at 22.5 μm/mL and 0.45 μg/ml represent safety threshold and treatment efficacy thresholds, respectively. The middle curve is a median concentration, and upper and lower curves represent 90% prediction intervals.

### DETAILED DESCRIPTION OF THE INVENTION

### Definitions

As used herein, the term “pharmaceutical composition” means a composition that is suitable for pharmaceutical use.

As used herein, the term “prevention” or “preventing” refers to avoidance of the stated disease in a subject who is not suffering from the stated disease.

As used herein, the term “treatment” or “treating” refers to alleviating the specified condition, eliminating or reducing the symptoms of the condition, slowing or eliminating the progression, invasion, or spread of the condition, and reducing or delaying the recurrence of the condition in a previously afflicted subject.

As used herein, the term “aqueous solution” refers to any solution comprising water or in which the solvent is water. Additionally, “aqueous solution” is used to describe solutions displaying commonalities to water or watery solutions, not limited to characteristics such as appearance, smell, colour, taste, viscosity, pH, absorbance, or physical state under particular temperatures.

As used herein, the term “lyophilization,” also known as freeze-drying or cryodesiccation, is a dehydration process which involves freezing the product without destroying the physical structure of the matter.

As used herein, the terms “lyophilized” and “freeze-dried” can be used interchangeably herein and refer to a condition and/or state of a sample, formulation, or product obtained by means of lyophilization.

As used interchangeably herein, the terms “lyophilized pharmaceutical composition” and “lyophilized composition” refer to a pharmaceutical composition in lyophilized form, as taught herein, for example a lyophilized powder.

As used herein, “reconstitution” refers to the process of restoring a dried, lyophilized, dehydrated, or concentrated matter to its original or liquid state by adding a solvent to the lyophilized matter, allowing the lyophilized matter to rehydrate, followed by agitating the mixture of the solvent and lyophilized matter. The reconstituted matter may be part of a product, formulation, sample, raw material, or any biological material but is certainly not limited to matter falling under the common definition of these terms. Reconstitution can be assessed visually with the naked eye. The lyophilized matter is deemed reconstituted when a homogeneous suspension is observed. In particular, a suspension with a cloudy appearance is considered suitably reconstituted.

As used herein, the term “self-administered” means administration by someone other than a healthcare professional, for example, a patient may administer the pharmaceutical composition to their self or someone else, other than a healthcare professional administering the pharmaceutical composition to the patient.

As used herein, the term “subject” or “patient” refers to a human.

As used herein, the term “diameter” refers to a spherical volume equivalent diameter.

As used herein, the term “Cmax” refers to maximum observed plasma concentration.

As used herein, the term “Ctau” refers to trough plasma concentration i.e. the concentration reached immediately before the next dose is administered.

As used herein the term “tmax” refers to time of maximum observed plasma concentration (tmax) of a substance after administration of that substance.

As used herein the term “Area under the curve” or “AUC” refers to area under the concentration (of substance in plasma)—time curve. AUC can be a measure of the integral of the instantaneous concentrations during a time interval and has the units mass*time/volume. AUC is typically calculated by the trapezoidal method (e.g., linear, linear-log). AUC is usually given for the time interval zero to infinity (AUC (0-inf)), and other time intervals are indicated (for example AUC (t1,t2) where t1 and t2 are the starting and finishing times for the interval).

As used herein, the term “about” generally means ±5%, ±10%, ±15%, or ±20% of the numerical value of the number with which it is being used. In an embodiment, the term “about” means ±10% of the numerical value of the number with which it is being used.

### DESCRIPTION OF THE EMBODIMENTS

In a first aspect, the present invention provides a method of preventing HIV in a human, comprising administering to said human a pharmaceutical composition comprising cabotegravir once every 15 to 24 weeks.

In a second aspect, the present invention provides a method of treating HIV in a human in need thereof, comprising administering to said human a pharmaceutical composition comprising cabotegravir once every 15 to 24 weeks.

The embodiments discussed below apply equally to the first and second aspects. Cabotegravir (3S, 11aR)-N-((2,4-difluorophenyl)methyl)-6-hydroxy-3-methyl-5,7-dioxo-2,3,5,7,11,11a-hexahydro (1,3) oxazolo (3,2-a)pyrido (1,2-d) pyrazine-8-carboxamide is described in U.S. Pat. No. 8,129,385 in example Z-9 which example is incorporated herein by reference. Cabotegravir is an integrase strand transfer inhibitor (INSTI) that exhibits subnanomolar potency and antiviral activity against a broad range of HIV-1 strains. Oral administration of cabotegravir has exhibited acceptable safety and tolerability profiles, a long half-life, and few drug-drug interactions. Cabotegravir has been demonstrated to be efficacious in treatment and prevention of HIV both in oral and parenteral dosage forms, see for instance, Margolis D A, Brinson C C, Eren J J, et al. 744 and Rilpivirine as Two Drug Oral Maintenance Therapy: LAI116482 (LATTE) Week 48 Results. 21^{st} Conference on Retroviruses and Opportunistic Infections (CROI); Mar. 3-6, 2014; Boston, MA, Margolis D A, Podzamczer D, Stellbrink H-J, et al. Cabotegravir+Rilpivirine as Long-Acting Maintenance Therapy: LATTE-2 Week 48 Results, 21^{st} International AIDS Conference; Jul. 18-22, 2016; Durban, South Africa, Abstract THAB0206LB. Levin: Conference reports for National AIDS Treatment Advocacy Project (NATAP); 2016, and Markowitz M, Frank I, Grant R, et al. ECLAIR: Phase 2A Safety and PK Study of Cabotegravir L A in HIV-Uninfected Men. Abstract presented at 23^{rd} CROI; Feb. 22-25, 2016; Boston, MA. Cabotegravir has been approved by the FDA for long-acting prevention of HIV infection dosed every two months; and in combination with Rilpivirine for long-acting treatment of HIV infection dosed once a month or once every two months.

### Cabotegravir is Represented by Compound A:

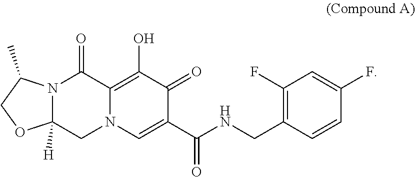 [SMILES: C[C@H]1CO[C@@H]2Cn3cc(C(=O)NCc4ccc(F)cc4F)c(=O)c(O)c3C(=O)N12]

In an embodiment of the invention, cabotegravir is present in the pharmaceutical composition as the free acid.

The dose of cabotegravir administered, which is the amount of cabotegravir in the composition for use in the invention, may be selected such that: the blood plasma concentration of cabotegravir in the human is kept above a trough plasma concentration (Ctau); or the blood plasma concentration of cabotegravir in the human is maintained at or above the Ctau of the approved 200 mg/mL cabotegravir regimen i.e. in the dosing regimen for APRETUDE®.

Trough plasma level (Ctau) refers to trough plasma concentration i.e. the concentration reached immediately before the next dose is administered. The Ctau value represents the lowest blood plasma level. The Ctau can be measured in any suitable way.

The present inventors have found that, to maintain an effective dosing regimen for treatment or prevention of HIV, the dose of cabotegravir, which is the amount of cabotegravir in the pharmaceutical composition for use in the invention, should maintain Ctau at a level higher than the 10th percentile of Ctau observed in the Phase 3 studies 201738 (HPTN 083) for people assigned male at birth (1.05 μg/mL) and 201739 (HPTN 084) for people assigned female at birth (1.39 μg/mL). The 10th percentile of Ctau observed in the Phase 3 studies mentioned are the sex-specific PrEP benchmarks which were achieved in at least 90% of participants or, the dose of cabotegravir needs to maintain the median and 10th percentile of Ctau higher than those from the approved 200 mg/ml intramuscular regimen of cabotegravir (APRETUDE®) in both people who were assigned male at birth and people who were assigned female at birth.

In an embodiment, the dose of cabotegravir administered is selected such that the Ctau is higher than the Ctau observed in the approved 200 mg/mL intramuscular regimen of cabotegravir in both people who were assigned male at birth and people who were assigned female at birth. In an embodiment, the dose of cabotegravir administered is selected such that the Ctau is higher than the Ctau observed in the dosing regimen of APRETUDE®. In an embodiment, the dose of cabotegravir administered is selected such that the Ctau is higher than the Ctau observed in the 10th percentile observed in the dosing regimen of APRETUDE®.

In an embodiment the dose of cabotegravir administered is selected such that the Ctau is at least 1 μg/mL i.e. the cabotegravir provides a Ctau of at least about 1 μg/mL. In another embodiment the cabotegravir provides a Ctau of at least 1.05 μg/mL. In another embodiment the human was assigned female at birth and the cabotegravir provides a Ctau of at least about 1.35 μg/mL. In another embodiment the human was assigned female at birth and the cabotegravir provides a Ctau of at least about 1.39 μg/mL. In another embodiment the human was assigned female at birth and the cabotegravir provides a Ctau of at least 1.39 μg/mL. In an embodiment the human was assigned male at birth and the cabotegravir provides a Ctau of at least about 1.05 μg/mL. In another embodiment the human was assigned male at birth and the cabotegravir provides a Ctau of at least 1.05 μg/mL. In an embodiment, the human was assigned female at birth and the cabotegravir provides a Ctau of at least about 1.39 μg/mL; or the human was assigned male at birth and the cabotegravir provides a Ctau of at least about 1.05 μg/mL.

In an embodiment, the Ctau levels mentioned above are achieved in at least 90% of humans being administered the dosing regimen.

The blood plasma levels of cabotegravir in a human may be kept above these Ctau levels because at lower levels the drug may no longer be effective, thereby increasing the risk of transmission of HIV infection, and may be suboptimal for treatment of HIV infected subjects. Plasma levels of cabotegravir may be kept at higher levels to avoid the development of HIV mutations, while maintaining a safety margin.

In an embodiment the use comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter.

In an embodiment the use comprises first administering a loading dose of the pharmaceutical composition to the human; 15 to 24 weeks after administering the loading dose, administering a maintenance dose of the pharmaceutical composition to the human; and then administering the maintenance dose every 15 to 24 weeks thereafter.

In an embodiment, the human is screened for HIV infection prior to receiving the loading dose. In an embodiment, the method is for prevention of HIV and human is screened for HIV infection prior to receiving the loading dose.

In an embodiment, the loading dose is a single dose of the pharmaceutical composition received at the initiation of treatment or prevention. In an embodiment, a first maintenance dose is administered to the human 2 to 6 weeks after they have been administered the loading dose. In another embodiment, a first maintenance dose is administered to the human 15 to 24 weeks after administering the loading dose. In an embodiment, the maintenance dose is then administered to the human every 15 to 24 weeks thereafter.

In an embodiment, the pharmaceutical composition used for the loading dose and the maintenance dose are the same pharmaceutical composition, but they may comprise different concentrations or amounts of cabotegravir. In an embodiment of the invention, the pharmaceutical composition used for the loading dose and the maintenance dose comprise the same concentration of cabotegravir. In an embodiment of the invention, the pharmaceutical composition used for the loading dose and the maintenance dose comprise the same concentration of cabotegravir, in this embodiment, the loading dose of cabotegravir is higher than the maintenance dose of cabotegravir. In an embodiment the loading dose and the maintenance dose are the same dose (for example, the loading dose and the maintenance dose are the same volume and each comprises the same pharmaceutical composition, with the same concentration and same amount of cabotegravir). In an embodiment the loading dose and maintenance dose are different doses (for example, the loading dose and the maintenance dose are different volumes of the same pharmaceutical composition with the same concentration of cabotegravir, or the loading dose and the maintenance dose are different volumes of different pharmaceutical compositions with different concentrations of cabotegravir). In an embodiment, the loading dose of cabotegravir is higher than the maintenance dose of cabotegravir (for example, the loading dose and the maintenance dose each comprise the same concentration of cabotegravir wherein the volume of the loading dose is greater than the volume of the maintenance dose, or the loading dose and the maintenance dose comprises different concentrations of cabotegravir wherein the volume of the loading dose is greater than the volume of the maintenance dose).

In another embodiment, the pharmaceutical composition used for the loading dose is a different pharmaceutical composition as the pharmaceutical composition used for the maintenance dose (for example, the pharmaceutical composition used for the loading dose has different excipient(s) from the pharmaceutical composition used for the maintenance dose).

In yet another embodiment, a loading dose comprises two different pharmaceutical compositions.

### Method of Administration

The pharmaceutical compositions described in the present invention are suitable for injection. In an embodiment the pharmaceutical composition is administered intramuscularly or subcutaneously via injection. In an embodiment the pharmaceutical composition is administered subcutaneously. In an embodiment the pharmaceutical composition is administered intramuscularly. In an embodiment, the pharmaceutical composition is administered in a gluteus muscle of the human. In a specific embodiment, the pharmaceutical composition is administered at the ventrogluteal or dorsogluteal site. In an embodiment, the pharmaceutical composition is administered in the gluteus medius, the gluteus maximus, or both the gluteus medius and the gluteus maximus. In an embodiment the pharmaceutical composition is administered in the gluteus medius. In an embodiment, the pharmaceutical composition is administered in the gluteus maximus.

### Loading Dose

In an embodiment the loading dose of the pharmaceutical composition comprises at least 600 mg of cabotegravir. In an embodiment the loading dose of the pharmaceutical composition comprises at least 1200 mg of cabotegravir. In an embodiment the loading dose of the pharmaceutical composition comprises at least 1400 mg of cabotegravir. In an embodiment the loading dose of the pharmaceutical composition comprises at least 1500 mg of cabotegravir. In another embodiment the loading dose of the pharmaceutical composition comprises at least 1600 mg of cabotegravir. In another embodiment the loading dose of the pharmaceutical composition comprises at least 1800 mg of cabotegravir. In another embodiment the loading dose of the pharmaceutical composition comprises at least 2000 mg of cabotegravir. In another embodiment the loading dose of the pharmaceutical composition comprises at least 2200 mg of cabotegravir. In another embodiment the loading dose of the pharmaceutical composition comprises at least 2400 mg of cabotegravir. In another embodiment the loading dose of the pharmaceutical composition comprises at least 2600 mg of cabotegravir. In another embodiment the loading dose of the pharmaceutical composition comprises at least 2800 mg of cabotegravir. In another embodiment the loading dose of the pharmaceutical composition comprises at least 3000 mg of cabotegravir. In an embodiment, the loading dose comprises about 600 mg to about 4000 mg of cabotegravir. In an embodiment, the loading dose comprises about 1200 mg to about 4000 mg of cabotegravir. In an embodiment the loading dose of the pharmaceutical composition comprises about 2000 mg to about 3500 mg of cabotegravir. In an alternative embodiment, the loading dose of the pharmaceutical composition comprises 3000 mg to about 3500 mg of cabotegravir. In an alternative embodiment, the loading dose of the pharmaceutical composition comprises 3100 mg to about 3300 mg of cabotegravir. In an alternative embodiment, the loading dose of the pharmaceutical composition comprises 3150 mg to about 3250 mg of cabotegravir. In an embodiment, the loading dose of the pharmaceutical composition comprises about 600 mg of cabotegravir. In an embodiment, the loading dose of the pharmaceutical composition comprises about 1600 mg of cabotegravir. In an embodiment, the loading dose of the pharmaceutical composition comprises about 2000 mg of cabotegravir. In an embodiment, the loading dose of the pharmaceutical composition comprises about 2132 mg of cabotegravir. In an embodiment, the loading dose of the pharmaceutical composition comprises about 2200 mg of cabotegravir. In an embodiment, the loading dose of the pharmaceutical composition comprises about 2400 mg of cabotegravir. In an embodiment, the loading dose of the pharmaceutical composition comprises about 3200 mg of cabotegravir. In an embodiment, the loading dose of the pharmaceutical composition comprises 600 mg of cabotegravir. In an embodiment, the loading dose of the pharmaceutical composition comprises 1600 mg of cabotegravir. In an embodiment, the loading dose of the pharmaceutical composition comprises 2200 mg of cabotegravir. In an embodiment, the loading dose of the pharmaceutical composition comprises 2400 mg of cabotegravir. In an embodiment, the loading dose of the pharmaceutical composition comprises 3200 mg of cabotegravir.

The loading dose may be administered in one injection or in two separate injections. In embodiments where the loading dose is administered in two separate injections, these injections are administered sequentially. In an embodiment where the loading dose is administered in two separate injections, the two separate injections are administered in the same site or in different sites. In an embodiment where the loading dose is administered in two separate injections, the two separate injections are administered sequentially in the same site. In another embodiment where the loading dose is administered in two separate injections, the two separate injections are administered sequentially in sites at least 2 cm apart (for example, one injection in left gluteus maximus and one injection in right gluteus maximus, or one injection in left gluteus medius and the other injection in right gluteus medius). In an embodiment, each of the two separate injections comprises the same amount of cabotegravir. In an embodiment, each of the two separate injections comprises different amounts of cabotegravir (for example, one injection comprises about 600 mg of cabotegravir and the other injection comprises about 1600 mg of cabotegravir).

In an embodiment where the loading dose is administered in two separate injections, the injections each contain the same pharmaceutical composition or different pharmaceutical compositions. In an embodiment where the loading dose is administered in two separate injections, the injections each contain the same pharmaceutical composition. In an embodiment where the loading dose is administered in two separate injections, the injections contain different pharmaceutical compositions.

In an embodiment, the loading dose of the pharmaceutical composition comprises 600 mg of cabotegravir wherein the loading dose is administered in one 2 mL injection.

In an embodiment, the loading dose of the pharmaceutical composition comprises 1600 mg of cabotegravir wherein the loading dose is administered in one 3 mL injection.

In an embodiment, the loading dose of the pharmaceutical composition comprises 2200 mg of cabotegravir wherein the loading dose is administered in two separate injections, wherein one injection comprises about 600 mg of cabotegravir and the other injection comprises about 1600 mg of cabotegravir.

In an embodiment, the loading dose of the pharmaceutical composition comprises 2400 mg of cabotegravir wherein the loading dose is administered in two separate injections, each comprising about 1200 mg of cabotegravir.

In an embodiment, the loading dose of the pharmaceutical composition comprises 3200 mg of cabotegravir wherein the loading dose is administered in two separate injections, each comprising about 1600 mg of cabotegravir.

In an embodiment, the loading dose is administered as a 1 mL injection. In an embodiment, the loading dose is administered as a 2 mL injection. In an embodiment the loading dose is administered as a 2.25 mL injection. In an embodiment, the loading dose is administered as a 3 mL injection.

In an embodiment, the loading dose of the pharmaceutical composition comprises 2132 mg of cabotegravir wherein the loading dose is administered in two separate 2 mL injections, each comprising about 1066 mg of cabotegravir.

In an embodiment, the loading dose of the pharmaceutical composition comprises 2200 mg of cabotegravir wherein the loading dose is administered in two separate injections, one comprising about 600 mg of cabotegravir in a 2 mL injection and the other comprising about 1600 mg cabotegravir in a 3 mL injection.

In an embodiment, the loading dose of the pharmaceutical composition comprises 2400 mg of cabotegravir wherein the loading dose is administered in two separate injections, each comprising about 1200 mg of cabotegravir in a 2.25 mL injection.

In an embodiment, the loading dose of the pharmaceutical composition comprises 3200 mg of cabotegravir wherein the loading dose is administered in two separate 3 mL injections, each comprising about 1600 mg of cabotegravir.

In another embodiment, the loading dose may refer to a first loading dose when one or more subsequent loading doses is administered at a later point in time (e.g., a second loading dose is administered 2-6 weeks after a first loading dose). Subsequent loading doses are as described in any of the preceding embodiments of a loading dose. In an embodiment, a first loading dose is followed by a second loading dose. In another embodiment, a first loading dose is followed by a second loading dose and a third loading dose. In another embodiment, a first loading dose is followed by a second loading dose, a third loading dose, and a fourth loading dose (for example, a second loading dose is administered 2-6 weeks after a first loading dose, a third loading dose is administered 6-11 weeks after the second loading dose, and a fourth loading dose is administered 6-11 weeks after the third loading dose).

### Maintenance Dose

In an embodiment, the maintenance dose of the pharmaceutical composition comprises at least 1000 mg of cabotegravir. In another embodiment the maintenance dose of the pharmaceutical composition comprises at least 1200 mg of cabotegravir. In an embodiment, the maintenance dose of the pharmaceutical composition comprises at least 1300 mg of cabotegravir. In an embodiment, the maintenance dose of the pharmaceutical composition comprises at least 1330 mg of cabotegravir. In another embodiment the maintenance dose of the pharmaceutical composition comprises at least 1500 mg of cabotegravir. In another embodiment the maintenance dose of the pharmaceutical composition comprises at least 1600 mg of cabotegravir. In an embodiment, the maintenance dose comprises about 1000 mg to about 1800 mg of cabotegravir. In an embodiment, the maintenance dose comprises about 1200 mg to about 1800 mg of cabotegravir. In another embodiment the maintenance dose of the pharmaceutical composition comprises about 1250 mg to about 1800 mg of cabotegravir. In another embodiment the maintenance dose of the pharmaceutical composition comprises about 1300 mg to about 1800 mg of cabotegravir. In another embodiment the maintenance dose of the pharmaceutical composition comprises about 1400 mg to about 1800 mg, about 1400 to about 1700 mg, about 1450 to about 1700 mg, about 1450 to about 1650 mg, about 1500 mg to about 1650 mg, or about 1550 mg to about 1650 mg of cabotegravir. In an embodiment the maintenance dose of the pharmaceutical composition comprises about 1550 mg of cabotegravir. In an embodiment the maintenance dose of the pharmaceutical composition comprises 1550 mg of cabotegravir. In an embodiment the maintenance dose of the pharmaceutical composition comprises about 1600 mg of cabotegravir. In an embodiment the maintenance dose of the pharmaceutical composition comprises 1600 mg of cabotegravir. In an embodiment the maintenance dose of the pharmaceutical composition comprises about 1650 mg of cabotegravir. In an embodiment the maintenance dose of the pharmaceutical composition comprises 1650 mg of cabotegravir.

In an embodiment, the maintenance dose is administered as a 1 mL injection. In an embodiment, the maintenance dose is administered as a 2 mL injection. In an embodiment, the maintenance dose is administered as a 3 mL injection.

In an embodiment the maintenance dose of the pharmaceutical composition comprises about 1066 mg of cabotegravir in a 2 mL injection, about 1332 mg of cabotegravir in a 2.5 mL injection or about 1600 mg of cabotegravir in a 3 mL injection.

In an embodiment the maintenance dose of the pharmaceutical composition comprises about 1066 mg of cabotegravir in a 2 mL injection.

In an embodiment the maintenance dose of the pharmaceutical composition comprises about 1600 mg of cabotegravir in a 3 mL injection.

In an embodiment, the first maintenance dose is administered 2 to 6 weeks after administering the loading dose and then every 15 to 24 weeks thereafter.

In an embodiment, the first maintenance dose is administered 2 to 6 weeks, or 3 to 4 weeks after the loading dose. In an embodiment the first maintenance dose is administered 1 month after the loading dose i.e. 3 to 5 weeks after the loading dose. In an embodiment, the first maintenance dose is administered about 3 weeks after the loading dose, about 4 weeks after the loading dose, or about 5 weeks after the loading dose. In an embodiment, the first maintenance dose is administered 3 weeks after the loading dose, 4 weeks after the loading dose or 5 weeks after the loading dose.

In another embodiment, the first maintenance dose is administered 6 to 11 weeks after administering the loading dose and then every 15 to 24 weeks thereafter.

In another embodiment, the first maintenance dose is administered 6 to 11 weeks, or 7 to 10 weeks after the loading dose. In an embodiment the first maintenance dose is administered about 2 months after the loading dose. In an embodiment the first maintenance dose is administered 2 months after the loading dose. In an embodiment, the first maintenance dose is administered about 7 weeks after the loading dose, about 8 weeks after the loading dose, about 9 weeks after the loading dose, or about 10 weeks after the loading dose. In an embodiment, the first maintenance dose is administered 7 weeks after the loading dose, 8 weeks after the loading dose, 9 weeks after the loading dose, or 10 weeks after the loading dose.

In another embodiment, the first maintenance dose is administered 15 to 19 weeks after administering the loading dose and then every 15 to 24 weeks thereafter.

In another embodiment, the first maintenance dose is administered 15 to 19 weeks, or 16 to 18 weeks after the loading dose. In an embodiment the first maintenance dose is administered about 4 months after the loading dose. In an embodiment the first maintenance dose is administered 4 months after the loading dose. In an embodiment, the first maintenance dose is administered about 16 weeks after the loading dose, about 17 weeks after the loading dose, or about 18 weeks after the loading dose. In an embodiment, the first maintenance dose is administered 16 weeks after the loading dose, 17 weeks after the loading dose or 18 weeks after the loading dose.

In an embodiment, the maintenance dose is administered once every 15 to 20, or every 15 to 19 weeks.

In an embodiment the maintenance dose is administered once every 4 months i.e. once every 16 to 18 weeks. In an embodiment, the maintenance dose is administered once every 16 weeks, every 17 weeks or every 18 weeks.

In an embodiment, the maintenance dose of the pharmaceutical composition comprises at least 1200 mg of cabotegravir and is administered every 4 months i.e. every 16 to 18 weeks. In an embodiment, the maintenance dose of the pharmaceutical composition comprises 1200 mg to 1800 mg of cabotegravir and is administered every 4 months i.e. every 16 to 18 weeks. In an embodiment, the maintenance dose of the pharmaceutical composition comprises about 1600 mg of cabotegravir and is administered every 4 months i.e. every 16 to 18 weeks.

In an embodiment, the method comprises first administering a loading dose of the pharmaceutical composition to the human wherein the pharmaceutical composition comprises at least 1500 mg of cabotegravir; 1 month or 3 to 5 weeks after administering the loading dose, administering a maintenance dose of the pharmaceutical composition comprising at least 1200 mg of cabotegravir to the human; and then administering the maintenance dose every 4 months or 16 to 18 weeks thereafter.

In an embodiment, the method comprises first administering a loading dose of the pharmaceutical composition to the human wherein the pharmaceutical composition comprises about 1200 mg to 4000 mg of cabotegravir; 1 month or 3 to 5 weeks after administering the loading dose, administering a maintenance dose of the pharmaceutical composition comprising 1200 to 1800 mg of cabotegravir to the human; and then administering the maintenance dose every 4 months or 16 to 18 weeks thereafter.

In an embodiment, the method comprises first administering a loading dose of the pharmaceutical composition to the human wherein the pharmaceutical composition comprises about 3000 mg to 3500 mg of cabotegravir; 1 month or 3 to 5 weeks after administering the loading dose, administering a maintenance dose of the pharmaceutical composition comprising 1500 to 1700 mg of cabotegravir to the human; and then administering the maintenance dose every 4 months or 16 to 18 weeks thereafter.

In an embodiment, the method comprises first administering a loading dose of the pharmaceutical composition to the human wherein the pharmaceutical composition comprises about 3200 mg of cabotegravir administered in two injections, each comprising 1600 mg of cabotegravir; 1 months or 3 to 5 weeks after administering the loading dose, administering a maintenance dose of the pharmaceutical composition comprising about 1600 mg of cabotegravir to the human; and then administering the maintenance dose every 4 months or 16 to 18 weeks thereafter.

In an embodiment, the method comprises first administering a first loading dose of the pharmaceutical composition(s) to the human wherein the pharmaceutical composition(s) comprises about 3200 mg of cabotegravir, wherein the first loading dose is optionally administered in two separate injections, each comprising 1600 mg of cabotegravir; 4 months or 16 to 18 weeks after administering the first loading dose, administering to the human a second loading dose of the pharmaceutical composition(s) comprising about 1600 mg of cabotegravir; 4 months or 16 to 18 weeks after administering the second loading dose, administering to the human a maintenance dose of the pharmaceutical composition(s) comprising about 1600 mg of cabotegravir; and then administering to the human the maintenance dose comprising about 1600 mg of cabotegravir every 4 months or 16 to 18 weeks thereafter.

In an embodiment, the method comprises first administering a first loading dose of the pharmaceutical composition(s) to the human wherein the pharmaceutical composition(s) comprises about 1600 mg of cabotegravir; 1 month or 3 to 5 weeks after administering the first loading dose, administering a second loading dose of the pharmaceutical composition(s) comprising about 1600 mg of cabotegravir to the human; 2 months or 7 to 10 weeks after administering the second loading dose, administering to the human a third loading dose of the pharmaceutical composition(s) comprising about 1600 mg of cabotegravir; four months or 16 to 18 weeks after administering the third loading dose, administering to the human a maintenance dose of the pharmaceutical composition(s) comprising about 1600 mg of cabotegravir; and then administering to the human the maintenance dose every 4 months or 16 to 18 weeks thereafter.

In an embodiment, the method comprises first administering a first loading dose of the pharmaceutical composition(s) to the human wherein the pharmaceutical composition(s) comprises about 2400 mg of cabotegravir, wherein the first loading dose is optionally administered in two separate injections, each comprising 1200 mg of cabotegravir; 2 months or 7 to 10 weeks after administering the first loading dose, administering to the human a second loading dose of the pharmaceutical composition(s) comprising about 1600 mg of cabotegravir; 4 months or 16 to weeks after administering the second loading dose, administering to the human a maintenance dose of the pharmaceutical composition(s) comprising about 1600 mg of cabotegravir; and then administering to the human the maintenance dose comprising about 1600 mg of cabotegravir every 4 months or 16 to 18 weeks thereafter.

In an embodiment, the method comprises first administering a first loading dose of the pharmaceutical composition(s) to the human wherein the pharmaceutical composition(s) comprises about 2200 mg of cabotegravir, wherein the first loading dose is optionally administered in two separate injections (e.g., one injection of about 600 mg cabotegravir and another injection of about 1600 mg cabotegravir); 2 months or 7 to 10 weeks after administering the first loading dose, administering to the human a second loading dose of the pharmaceutical composition(s) comprising about 1600 mg of cabotegravir; 2 months or 7 to 10 weeks after administering the second loading dose, administering to the human a third loading dose of the pharmaceutical composition(s) comprising about 1600 mg of cabotegravir; four months or 16 to 18 weeks after administering the third loading dose, administering to the human a maintenance dose of the pharmaceutical composition comprising about 1600 mg of cabotegravir; and then administering to the human the maintenance dose comprising about 1600 mg of cabotegravir every 4 months or 16 to 18 weeks thereafter.

In an embodiment, the method comprises first administering a first loading dose of the pharmaceutical composition(s) to the human wherein the pharmaceutical composition(s) comprises about 600 mg of cabotegravir; 1 month or 3 to 5 weeks after administering the first loading dose, administering a second loading dose of the pharmaceutical composition(s) comprising about 1600 mg of cabotegravir to the human; 2 months or 7 to 10 weeks after administering the second loading dose, administering to the human a third loading dose of the pharmaceutical composition(s) comprising about 1600 mg of cabotegravir; 4 months or 16 to 18 weeks after administering the third loading dose, administering to the human a maintenance dose of the pharmaceutical composition(s) comprising about 1600 mg of cabotegravir; and then administering to the human the maintenance dose every 4 months or 16 to 18 weeks thereafter.

In an embodiment, the method comprises first administering a first loading dose of the pharmaceutical composition(s) to the human wherein the pharmaceutical composition(s) comprises about 600 mg of cabotegravir; 1 month or 3 to 5 weeks after administering the first loading dose, administering a second loading dose of the pharmaceutical composition(s) comprising about 600 mg of cabotegravir to the human; 2 months or 7 to 10 weeks after administering the second loading dose, administering to the human a third loading dose of the pharmaceutical composition(s) comprising about 1600 mg of cabotegravir; 2 months or 7 to 10 weeks after administering the third loading dose, administering to the human a fourth loading dose of the pharmaceutical composition(s) comprising about 1600 mg of cabotegravir; 4 months or 16 to 18 weeks after administering the fourth loading dose, administering to the human a maintenance dose of the pharmaceutical composition comprising about 1600 mg of cabotegravir; and then administering to the human the maintenance dose every 4 months or 16 to 18 weeks thereafter.

### Oral Lead-In and Oral Bridging

In an embodiment, prior to the loading dose, an oral lead-in is given to the human to assess tolerability of cabotegravir. In an embodiment, the oral lead-in is taken once daily for about 1 month i.e. at least 28 days prior to administering the loading dose. In an embodiment the oral lead-in is a tablet of cabotegravir taken once daily. In an embodiment, the tablet comprises cabotegravir sodium. In an embodiment, the tablet comprises about 30 mg of cabotegravir sodium. In an embodiment, the tablet comprises 30 mg of cabotegravir (equivalent to 31.62 mg of cabotegravir sodium). In an embodiment, the loading dose is administered on the last day of oral lead-in. In an embodiment, the loading dose is administered within 3 days of stopping the oral lead in.

In an embodiment, the maintenance dose is discontinued and replaced with oral tablets. In an embodiment, the oral tablet is taken once daily. In an embodiment the oral tablet is a tablet of cabotegravir taken once daily. In an embodiment, the tablet comprises cabotegravir sodium. In an embodiment, the tablet comprises about 30 mg of cabotegravir sodium. In an embodiment, the tablet comprises 30 mg of cabotegravir (equivalent to 31.62 mg of cabotegravir sodium). In an embodiment, the human restarts the maintenance dose after taking daily oral tablets for a maximum of 4 months or a maximum of 16 to 18 weeks. In this embodiment, the maintenance dose is discontinued and replaced with oral tablets of cabotegravir taken daily for up to 4 months or up to 16 to 18 weeks and then the maintenance dose is reinitiated and taken once every 15 to 20 weeks thereafter.

In a separate embodiment, the human is taking daily oral tablets for more than 4 months or more than 16 to 18 weeks, in this embodiment the loading dose is taken before the maintenance dose is reinitiated. In this embodiment, the maintenance dose is discontinued and replaced with oral tablets of cabotegravir taken daily for at least 4 months or at least 16 to 18 weeks and then the loading dose is administered to the human; 2 to 6 weeks after administering the loading dose, administering the maintenance dose of the pharmaceutical composition to the human; and then administering the maintenance dose every 15 to 24 weeks thereafter.

### Pharmaceutical Compositions

Pharmaceutical compositions used in the methods of the present invention comprise cabotegravir, as described herein.

In an embodiment, the pharmaceutical composition contains about 100 to about 800 mg/mL of cabotegravir. In a further embodiment, the pharmaceutical composition contains about 200 mg/mL to about 700 mg/mL, from about 300 mg/mL to about 650 mg/mL, from about 400 mg/mL to about 600 mg/mL, from about 450 mg/mL to about 600 mg/mL, from about 500 mg/mL to about 600 mg/mL, from about 550 mg/ml to about 600 mg/mL, about 200 mg/mL, about 400 mg/mL, about 500 mg/mL, or about 533 mg/ml of cabotegravir. In an embodiment the pharmaceutical composition contains about 200 mg/ml of cabotegravir. In an embodiment the pharmaceutical composition contains 200 mg/ml of cabotegravir. In an embodiment the pharmaceutical composition contains about 400 mg/ml of cabotegravir. In a still further embodiment the pharmaceutical composition contains 400 mg/mL of cabotegravir. In a further embodiment, the pharmaceutical composition contains about 533 mg/ml of cabotegravir. In a still further embodiment the pharmaceutical composition contains 533 mg/mL of cabotegravir.

In an embodiment, the pharmaceutical composition has been reconstituted from a lyophilized powder and the pharmaceutical composition contains, after reconstitution, about 100 to about 800 mg/mL of cabotegravir. In a further embodiment, the pharmaceutical composition contains, after reconstitution, about 200 mg/ml to about 700 mg/mL, from about 300 mg/mL to about 650 mg/mL, from about 400 mg/mL to about 600 mg/mL, about 400, about 500, or about 533 mg/mL of cabotegravir. In an embodiment the pharmaceutical composition contains, after reconstitution, about 400 mg/ml of cabotegravir. In a further embodiment, the pharmaceutical composition contains, after reconstitution, about 533 mg/mL of cabotegravir.

Wetting agents or surfactants are compounds that, when dissolved in a liquid, can reduce the surface tension of a gas, liquid, or solid surface in that liquid. They are often amphiphilic and may aid in wetting and enhance manufacturability of the drug product. In addition, a surfactant may additionally impart long-term product stability by steric or electrostatic repulsion. Non-ionic surfactants are preferred over ionic surfactants as they are generally non-toxic, non-irritating, and inert. Examples of surfactants include, but are not limited to, polysorbate 20 (Tween-20), polysorbate 80 (Tween-80), sorbitan monolaurate (Span-20), sorbitan monooleate (Span-80), poloxamer 188 (Kolliphor P188), poloxamer 338 (Kolliphor P338), and poloxamer 407 (Kolliphor P407).

In an embodiment, the pharmaceutical composition used in the methods of the invention comprises polysorbate 80 (PS80) as the wetting agent.

PS80 (IUPAC name: polyoxyethylene (20) sorbitan monooleate; CAS No. 9005-65-6) is a nonionic surfactant and emulsifier derived from polyethoxylated sorbitan and oleic acid. The hydrophilic groups in PS80 are polyethers also known as polyoxyethylene groups, which are polymers of ethylene oxide. In the nomenclature of polysorbates, the numeric designation following “polysorbate” (e.g., “polysorbate 80”) refers to the lipophilic group, in this case, the oleic acid. The structure of PS80 is provided by formula (II):

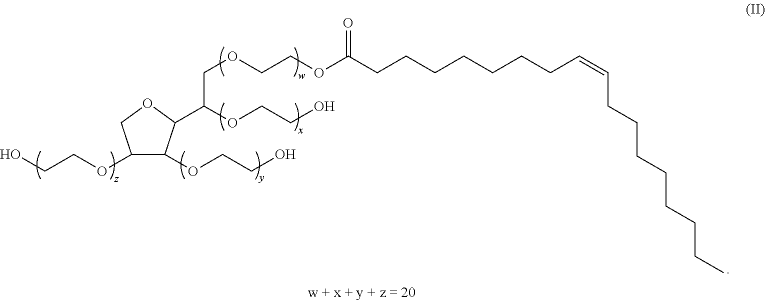

In an embodiment, the pharmaceutical composition contains from about 0.1 mg/mL to about 150 mg/ml of the wetting agent. In a further embodiment, the pharmaceutical composition contains from about 1 mg/mL to about 80 mg/mL, from about 2 mg/mL to about 40 mg/mL, from about 2.5 mg/mL to about 6 mg/mL, or from about 2.5 mg/mL to about 5 mg/ml of the wetting agent.

In an embodiment, the pharmaceutical composition contains about, in mg/ml, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 of the wetting agent. In another embodiment, the pharmaceutical composition contains about 3.0 mg/ml of the wetting agent. In another embodiment, the pharmaceutical composition contains about 4.0 mg/ml of the wetting agent. In another embodiment, the pharmaceutical composition contains about 5.3 mg/ml of the wetting agent.

In an embodiment, the pharmaceutical composition has been reconstituted from a lyophilized powder and contains from about 0.1 mg/mL to about 150 mg/ml of the wetting agent. In a further embodiment, the pharmaceutical composition contains, after reconstitution, from about 1 mg/mL to about 80 mg/mL, from about 2 mg/ml to about 40 mg/mL, from about 2.5 mg/mL to about 6 mg/mL, or from about 2.5 mg/mL to about 5 mg/ml of the wetting agent.

In an embodiment, the pharmaceutical composition has been reconstituted from a lyophilized powder and contains about, in mg/ml, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 of the wetting agent. In another embodiment, the pharmaceutical composition contains, after reconstitution, about 3.0 mg/ml of the wetting agent. In another embodiment, the pharmaceutical composition contains, after reconstitution, about 4.0 mg/ml of the wetting agent. In another embodiment, the pharmaceutical composition contains, after reconstitution, about 5.3 mg/ml of the wetting agent.

In an embodiment, the pharmaceutical composition contains about 0.1 mg to about 900 mg of the wetting agent. In another embodiment, the pharmaceutical composition contains about 0.5 mg to about 200 mg of the wetting agent. In another embodiment, the pharmaceutical composition comprises about 1.0 mg to about 100 mg of the wetting agent. In another embodiment, the pharmaceutical composition contains about 2.0 mg to about 50 mg of the wetting agent. In another embodiment, the pharmaceutical composition contains about, in mg, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0 of the wetting agent. In an embodiment, the pharmaceutical composition comprises about 5.9 mg of the wetting agent. In an embodiment, the pharmaceutical composition comprises about 8.0 mg of the wetting agent.

In an embodiment, a weight ratio of the wetting agent to cabotegravir is in a range of from 1:10 to 1:400. In another embodiment, the weight ratio of the wetting agent to cabotegravir is in a range of from 1:50 to 1:200. In another embodiment, the weight ratio of the wetting agent to cabotegravir is in a range of from 1:100 to 1:150. In another embodiment, the weight ratio of the wetting agent to cabotegravir is about 1:100, about 1:101, about 1:102, about 1:103, about 1:104, about 1:105, about 1:106, about 1:107, about 1:108, about 1:109, about 1:110, about 1:111, about 1:112, about 1:113, about 1:114, about 1:115, about 1:116, about 1:117, about 1:118, about 1:119, about 1:120, about 1:121, about 1:122, about 1:123, about 1:124, about 1:125, about 1:126, about 1:127, about 1:128, about 1:129, about 1:130, about 1:131, about 1:132, about 1:133, about 1:134, about 1:135, about 1:136, about 1:137, about 1:138, about 1:139, about 1:140, about 1:141, about 1:142, about 1:143, about 1:144, about 1:145, about 1:146, about 1:147, about 1:148, about 1:149, or about 1:150. In another embodiment, the weight ratio of the wetting agent to cabotegravir is about 1:100. In another embodiment, the weight ratio of the wetting agent to cabotegravir is about 1:105. In another embodiment, the weight ratio of the wetting agent to cabotegravir is about 1:110. In another embodiment, the weight ratio of the wetting agent to cabotegravir is about 1:115. In another embodiment, the weight ratio of the wetting agent to cabotegravir is about 1:136.

Stabilizers are components added to help preserve critical product attributes throughout shelf life. In the case of suspensions, stabilizers can be used to induce charge effects, add steric stabilization, increase viscosity of the vehicle, etc. These factors can preserve particle size, product resuspendability, and/or improve manufacturability. Examples of stabilizers include, but are not limited to, sodium carboxymethylcellulose (CMC), polyethylene glycol 3350, polyethylene glycol 4000, povidone K12, and povidone K17.

In an embodiment, the pharmaceutical composition of the invention comprises sodium CMC as the stabilizer.

In an embodiment, the pharmaceutical composition contains about 0.1 to about 150 mg/ml of the stabilizer. In a further embodiment, the pharmaceutical composition contains about 1 mg/mL to about 25 mg/mL, from about 2 mg/mL to about 15 mg/mL, from about 2 mg/mL to about 10 mg/mL, or from about 3 mg/mL to about 10 mg/mL of the stabilizer.

In an embodiment, the pharmaceutical composition contains about, in mg/ml, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 of the stabilizer. In another embodiment, the pharmaceutical composition contains about 3.7 mg/ml of the stabilizer. In another embodiment, the pharmaceutical composition contains about 5.0 mg/ml of the stabilizer. In another embodiment, the pharmaceutical composition contains about 6.7 mg/mL of the stabilizer.

In an embodiment, the pharmaceutical composition has been reconstituted from a lyophilized powder and contains about 0.1 to about 150 mg/mL of the stabilizer. In a further embodiment, the pharmaceutical composition contains, after reconstitution, about 1 mg/ml to about 25 mg/mL, from about 2 mg/mL to about 15 mg/mL, from about 2 mg/ml to about 10 mg/mL, or from about 3 mg/mL to about 10 mg/ml of the stabilizer.

In an embodiment, the pharmaceutical composition has been reconstituted from a lyophilized powder and contains about, in mg/ml, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 of the stabilizer. In another embodiment, the pharmaceutical composition contains, after reconstitution, about 3.7 mg/mL of the stabilizer. In another embodiment, the pharmaceutical composition contains, after reconstitution, about 5.0 mg/ml of the stabilizer. In another embodiment, the pharmaceutical composition contains, after reconstitution, about 6.7 mg/ml of the stabilizer.

In an embodiment, the pharmaceutical composition contains about 0.1 mg to about 300 mg of the stabilizer. In another embodiment, the pharmaceutical composition contains about 1.0 mg to about 200 mg of the stabilizer. In another embodiment, the pharmaceutical composition comprises about 2.0 mg to about 100 mg of the stabilizer. In another embodiment, the pharmaceutical composition contains about 4.0 mg to about 50 mg of the stabilizer. In another embodiment, the pharmaceutical composition contains about, in mg, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0 of the stabilizer. In an embodiment, the pharmaceutical composition comprises about 7.4 mg of the stabilizer. In an embodiment, the pharmaceutical composition comprises about 10.0 mg of the stabilizer.

In an embodiment, a weight ratio of the stabilizer to cabotegravir is in a range of from 1:10 to 1:400. In another embodiment, the weight ratio of the stabilizer to cabotegravir is in a range of from 1:40 to 1:200. In another embodiment, the weight ratio of the stabilizer to cabotegravir is in a range of from 1:70 to 1:120. In another embodiment, the weight ratio of the stabilizer to cabotegravir is about 1:70, about 1:71, about 1:72, about 1:73, about 1:74, about 1:75, about 1:76, about 1:77, about 1:78, about 1:79, about 1:80, about 1:81, about 1:82, about 1:83, about 1:84, about 1:85, about 1:86, about 1:87, about 1:88, about 1:89, about 1:90, about 1:91, about 1:92, about 1:93, about 1:94, about 1:95, about 1:96, about 1:97, about 1:98, about 1:99, about 1:100, about 1:101, about 1:102, about 1:103, about 1:104, about 1:105, about 1:106, about 1:107, about 1:108, about 1:109, about 1:110, about 1:111, about 1:112, about 1:113, about 1:114, about 1:115, about 1:116, about 1:117, about 1:118, about 1:119, or about 1:120. In another embodiment, the weight ratio of the stabilizer to cabotegravir is about 1:80. In another embodiment, the weight ratio of the stabilizer to cabotegravir is about 1:100. In another embodiment, the weight ratio of the stabilizer to cabotegravir is about 1:101. In another embodiment, the weight ratio of the stabilizer to cabotegravir is about 1:102. In another embodiment, the weight ratio of the stabilizer to cabotegravir is about 1:103. In another embodiment, the weight ratio of the stabilizer to cabotegravir is about 1:104. In another embodiment, the weight ratio of the stabilizer to cabotegravir is about 1:105. In another embodiment, the weight ratio of the stabilizer to cabotegravir is about 1:106. In another embodiment, the weight ratio of the stabilizer to cabotegravir is about 1:107. In another embodiment, the weight ratio of the stabilizer to cabotegravir is about 1:108.

Tonicity adjusters act to provide and maintain a stable tonicity for the pharmaceutical composition disclosed herein. In some embodiments, tonicity adjusters also function as a non-aqueous solvent, a solubilizer, and/or a stabilizer. In such instances, tonicity adjusters may be used at concentrations higher than needed for tonicity if their primary purpose is stabilization or may be used at concentrations higher than needed for stabilization if their primary purpose is tonicity adjustment.

In some embodiments, the tonicity adjuster is a pharmaceutically acceptable inorganic chloride, e.g., potassium chloride, sodium chloride, magnesium chloride or calcium chloride. In yet other embodiments, the tonicity adjuster is a saccharide such as mannitol, sorbitol, lactose, trehalose, raffinose, dextrose, maltose, galactose, sucrose, or polysucrose. In still further aspects, the tonicity adjuster is mannitol. In other aspects, the tonicity adjuster is a non-aqueous polar aprotic or protic materials such as polyethylene glycol, N,N-dimethylacetamide, N-methyl pyrrolidone, glycerol, propylene glycol, ethanol, t-butyl alcohol, benzyl alcohol, benzyl benzoate, dimethyl sulfoxide, or glycerol. In further aspects, the tonicity adjuster is a polymer such as polyethylene glycol, polygalacturonic acid, galacturonic acid, polyvinylpyrrolidine (PVP), for example, PEG 300, PEG 400, PEG 3350, PEG 6000, or PEG 8000. In still other aspects, the tonicity adjuster is an amino acid such as lysine, arginine, glycine, methionine, or other amino acids. In yet further aspects, the tonicity adjuster is a cyclodextrin such as dextran, Ficoll, and polyvinylpyrrolidone, and other similar excipients and combinations of these agents.

In an embodiment, the pharmaceutical composition of the invention comprises mannitol as the tonicity adjuster.

In an embodiment, the pharmaceutical composition contains about 0.1 to about 250 mg/ml of the tonicity adjuster. In a further embodiment, the pharmaceutical composition contains about 1 mg/mL to about 150 mg/mL, from about 10 mg/mL to about 125 mg/mL, from about 15 mg/mL to about 60 mg/mL, from about 15 mg/mL to about 50 mg/mL, from about 20 mg/mL to about 50 mg/mL, or from about 20 mg/ml to about 40 mg/ml of the tonicity adjuster.

In an embodiment, the pharmaceutical composition contains about, in mg/mL, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0 of the tonicity adjuster. In another embodiment, the pharmaceutical composition contains about 25.9 mg/ml of the tonicity adjuster. In another embodiment, the pharmaceutical composition contains about 35.0 mg/ml of the tonicity adjuster. In another embodiment, the pharmaceutical composition contains about 46.6 mg/ml of the tonicity adjuster.

In an embodiment, the pharmaceutical composition has been reconstituted from a lyophilized powder and contains about 0.1 to about 250 mg/mL of the tonicity adjuster. In a further embodiment, the pharmaceutical composition contains, after reconstitution, about 1 mg/ml to about 150 mg/mL, from about 10 mg/mL to about 125 mg/mL, from about 15 mg/mL to about 60 mg/mL, from about 15 mg/mL to about 50 mg/mL, from about 20 mg/mL to about 50 mg/mL, or from about 20 mg/mL to about 40 mg/ml of the tonicity adjuster.

In an embodiment, the pharmaceutical composition has been reconstituted from a lyophilized powder and contains about, in mg/ml, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, or 50.0 of the tonicity adjuster. In another embodiment, the pharmaceutical composition contains, after reconstitution, about 25.9 mg/ml of the tonicity adjuster. In another embodiment, the pharmaceutical composition contains, after reconstitution, about 35.0 mg/ml of the tonicity adjuster. In another embodiment, the pharmaceutical composition contains, after reconstitution, about 46.6 mg/ml of the tonicity adjuster.

In an embodiment, the pharmaceutical composition contains about 0.1 mg to about 400 mg of the tonicity adjuster. In another embodiment, the pharmaceutical composition contains about 1.0 mg to about 300 mg of the tonicity adjuster. In another embodiment, the pharmaceutical composition comprises about 10 mg to about 100 mg of the tonicity adjuster. In another embodiment, the pharmaceutical composition contains about 35 mg to about 80 mg of the tonicity adjuster. In another embodiment, the pharmaceutical composition contains about, in mg, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, 50.0, 50.1, 50.2, 50.3, 50.4, 50.5, 50.6, 50.7, 50.8, 50.9, 51.0, 51.1, 51.2, 51.3, 51.4, 51.5, 51.6, 51.7, 51.8, 51.9, 52.0, 52.1, 52.2, 52.3, 52.4, 52.5, 52.6, 52.7, 52.8, 52.9, 53.0, 53.1, 53.2, 53.3, 53.4, 53.5, 53.6, 53.7, 53.8, 53.9, 54.0, 54.1, 54.2, 54.3, 54.4, 54.5, 54.6, 54.7, 54.8, 54.9, 55.0, 55.1, 55.2, 55.3, 55.4, 55.5, 55.6, 55.7, 55.8, 55.9, 56.0, 56.1, 56.2, 56.3, 56.4, 56.5, 56.6, 56.7, 56.8, 56.9, 57.0, 57.1, 57.2, 57.3, 57.4, 57.5, 57.6, 57.7, 57.8, 57.9, 58.0, 58.1, 58.2, 58.3, 58.4, 58.5, 58.6, 58.7, 58.8, 58.9, 59.0, 59.1, 59.2, 59.3, 59.4, 59.5, 59.6, 59.7, 59.8, 59.9, 60.0, 60.1, 60.2, 60.3, 60.4, 60.5, 60.6, 60.7, 60.8, 60.9, 61.0, 61.1, 61.2, 61.3, 61.4, 61.5, 61.6, 61.7, 61.8, 61.9, 62.0, 62.1, 62.2, 62.3, 62.4, 62.5, 62.6, 62.7, 62.8, 62.9, 63.0, 63.1, 63.2, 63.3, 63.4, 63.5, 63.6, 63.7, 63.8, 63.9, 64.0, 64.1, 64.2, 64.3, 64.4, 64.5, 64.6, 64.7, 64.8, 64.9, 65.0, 65.1, 65.2, 65.3, 65.4, 65.5, 65.6, 65.7, 65.8, 65.9, 66.0, 66.1, 66.2, 66.3, 66.4, 66.5, 66.6, 66.7, 66.8, 66.9, 67.0, 67.1, 67.2, 67.3, 67.4, 67.5, 67.6, 67.7, 67.8, 67.9, 68.0, 68.1, 68.2, 68.3, 68.4, 68.5, 68.6, 68.7, 68.8, 68.9, 69.0, 69.1, 69.2, 69.3, 69.4, 69.5, 69.6, 69.7, 69.8, 69.9, 70.0, 70.1, 70.2, 70.3, 70.4, 70.5, 70.6, 70.7, 70.8, 70.9, 71.0, 71.1, 71.2, 71.3, 71.4, 71.5, 71.6, 71.7, 71.8, 71.9, 72.0, 72.1, 72.2, 72.3, 72.4, 72.5, 72.6, 72.7, 72.8, 72.9, 73.0, 73.1, 73.2, 73.3, 73.4, 73.5, 73.6, 73.7, 73.8, 73.9, 74.0, 74.1, 74.2, 74.3, 74.4, 74.5, 74.6, 74.7, 74.8, 74.9, 75.0, 75.1, 75.2, 75.3, 75.4, 75.5, 75.6, 75.7, 75.8, 75.9, 76.0, 76.1, 76.2, 76.3, 76.4, 76.5, 76.6, 76.7, 76.8, 76.9, 77.0, 77.1, 77.2, 77.3, 77.4, 77.5, 77.6, 77.7, 77.8, 77.9, 78.0, 78.1, 78.2, 78.3, 78.4, 78.5, 78.6, 78.7, 78.8, 78.9, 79.0, 79.1, 79.2, 79.3, 79.4, 79.5, 79.6, 79.7, 79.8, 79.9, or 80.0 of the tonicity adjuster. In an embodiment, the pharmaceutical composition comprises about 51.8 mg of the tonicity adjuster. In an embodiment, the pharmaceutical composition comprises about 70 mg of the tonicity adjuster. In an embodiment, the pharmaceutical composition comprises about 105 mg of the tonicity adjuster. In an embodiment, the pharmaceutical composition comprises about 140 mg of the tonicity adjuster. In an embodiment, the pharmaceutical composition comprises about 175 mg of the tonicity adjuster. In an embodiment, the pharmaceutical composition comprises about 210 mg of the tonicity adjuster. In an embodiment, the pharmaceutical composition comprises about 245 mg of the tonicity adjuster. In an embodiment, the pharmaceutical composition comprises about 280 mg of the tonicity adjuster.

In an embodiment, a weight ratio of the tonicity adjuster to cabotegravir is in a range of from 1:1 to 1:100. In another embodiment, the weight ratio of the tonicity adjuster to cabotegravir is in a range of from 1:5 to 1:50. In another embodiment, the weight ratio of the tonicity adjuster to cabotegravir is in a range of from 1:8 to 1:25. In another embodiment, the weight ratio of the tonicity adjuster to cabotegravir is about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, or about 1:25. In another embodiment, the weight ratio of the tonicity adjuster to cabotegravir is about 1:8. In another embodiment, the weight ratio of the tonicity adjuster to cabotegravir is about 1:9. In another embodiment, the weight ratio of the tonicity adjuster to cabotegravir is about 1:10. In another embodiment, the weight ratio of the tonicity adjuster to cabotegravir is about 1:11. In another embodiment, the weight ratio of the tonicity adjuster to cabotegravir is about 1:12. In another embodiment, the weight ratio of the tonicity adjuster to cabotegravir is about 1:13. In another embodiment, the weight ratio of the tonicity adjuster to cabotegravir is about 1:14. In another embodiment, the weight ratio of the tonicity adjuster to cabotegravir is about 1:15.

In an embodiment, the pharmaceutical composition of the present invention comprises particles of crystalline cabotegravir. In an embodiment, cabotegravir particles of the pharmaceutical composition have an X50 value greater than or equal to 2.5 μm and less than or equal to 10 μm (i.e., 2.5 μm≤X50≤10 μm). Particle size distribution may be measured by any suitable method, for example, by laser diffraction as described in the Examples section herein.

As used herein, X50 (or “the X50 value”) is the cabotegravir particle diameter, in microns, at which 50% by volume of the cabotegravir particles have a smaller diameter and 50% by volume have a larger diameter, also known as the mass median diameter (MMD) or the median of the particle size distribution by volume.

As used herein, X90 (or “the X90 value”) is the cabotegravir particle diameter, in microns, at which 90% by volume of the cabotegravir particles have a smaller diameter and 10% by volume have a larger diameter.

As used herein, X10 (or “the X10 value”) is the cabotegravir particle diameter, in microns, at which 10% by volume of the cabotegravir particles have a smaller diameter and 90% by volume have a larger diameter.

In an embodiment, the pharmaceutical composition has a particle size distribution by volume such that 90% of the cabotegravir particles have a particle diameter less than or equal to 25 μm (i.e., X90 is 25 μm). In an embodiment, cabotegravir particles of the pharmaceutical composition have an X90 value greater than or equal to 5 μm and less than or equal to 25 μm (i.e., 5 μm≤X90≤25 μm). In an embodiment, the pharmaceutical composition has a particle size distribution by volume such that 90% of the cabotegravir particles have a particle diameter less than or equal to 20 μm (i.e., X90 is 20 μm). In an embodiment, cabotegravir particles of the pharmaceutical composition have an X90 value greater than or equal to 6 μm and less than or equal to 20 μm (i.e., 6 μm≤X90≤20 μm). In an embodiment, the pharmaceutical composition has a particle size distribution by volume such that 90% of the cabotegravir particles have a particle diameter less than or equal to 18 μm (i.e., X90 is 18 μm). In an embodiment, cabotegravir particles of the pharmaceutical composition have an X90 value greater than or equal to 7 μm and less than or equal to 18 μm (i.e., 7 μm≤X90≤18 μm).

In an embodiment, the pharmaceutical composition has a particle size distribution by volume such that 90% of the cabotegravir particles (X90) have a particle diameter smaller than or equal to 7.0 μm, 7.1 μm, 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, 10.0 μm, 10.1 μm, 10.2 μm, 10.3 μm, 10.4 μm, 10.5 μm, 10.6 μm, 10.7 μm, 10.8 μm, 10.9 μm, 11.0 μm, 11.1 μm, 11.2 μm, 11.3 μm, 11.4 μm, 11.5 μm, 11.6 μm, 11.7 μm, 11.8 μm, 11.9 μm, 12.0 μm, 12.1 μm, 12.2 μm, 12.3 μm, 12.4 μm, 12.5 μm, 12.6 μm, 12.7 μm, 12.8 μm, 12.9 μm, 13.0 μm, 13.1 μm, 13.2 μm, 13.3 μm, 13.4 μm, 13.5 μm, 13.6 μm, 13.7 μm, 13.8 μm, 13.9 μm, 14.0 μm, 14.1 μm, 14.2 μm, 14.3 μm, 14.4 μm, 14.5 μm, 14.6 μm, 14.7 μm, 14.8 μm, 14.9 μm, 15.0 μm, 15.1 μm, 15.2 μm, 15.3 μm, 15.4 μm, 15.5 μm, 15.6 μm, 15.7 μm, 15.8 μm, 15.9 μm, 16.0 μm, 16.1 μm, 16.2 μm, 16.3 μm, 16.4 μm, 16.5 μm, 16.6 μm, 16.7 μm, 16.8 μm, 16.9 μm, 17.0 μm, 17.1 μm, 17.2 μm, 17.3 μm, 17.4 μm, 17.5 μm, 17.6 μm, 17.7 μm, 17.8 μm, 17.9 μm, or 18.0 μm. In another embodiment, the pharmaceutical composition has a particle size distribution by volume such that 90% of the cabotegravir particles (X90) have a particle diameter smaller than or equal to 9 μm (i.e., X90 is 9 μm). In another embodiment, the pharmaceutical composition has a particle size distribution by volume such that 90% of the cabotegravir particles (X90) have a particle diameter smaller than or equal to 14 μm (i.e., X90 is 14 μm). In another embodiment, the pharmaceutical composition has a particle size distribution by volume such that 90% of the cabotegravir particles (X90) have a particle diameter smaller than or equal to 17 μm (i.e., X90 is 17 μm).

In an embodiment, the pharmaceutical composition has a particle size distribution by volume such that 50% of the cabotegravir particles have a particle diameter less than or equal to 10 μm (i.e., X50 is 10 μm). In an embodiment, cabotegravir particles of the pharmaceutical composition have an X50 value greater than or equal to 2.5 μm and less than or equal to 10 μm (i.e., 2.5 μm≤X50≤10 μm). In an embodiment, the pharmaceutical composition has a particle size distribution by volume such that 50% of the cabotegravir particles have a particle diameter less than or equal to 8.5 μm (i.e., X50 is 8.5 μm). In an embodiment, cabotegravir particles of the pharmaceutical composition have an X50 value greater than or equal to 3 μm and less than or equal to 8.5 μm (i.e., 3 μm≤X50≤8.5 μm). In an embodiment, the pharmaceutical composition has a particle size distribution by volume such that 50% of the cabotegravir particles have a particle diameter less than or equal to 8 μm (i.e., X50 is 8 μm). In an embodiment, cabotegravir particles of the pharmaceutical composition have an X50 value greater than or equal to 3.5 μm and less than or equal to 8 μm (i.e., 3.5 μm≤X50≤8 μm).

In an embodiment, the pharmaceutical composition has a particle size distribution by volume such that 50% of the cabotegravir particles (X50) have a particle diameter smaller than or equal to 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 5.1 μm, 5.2 μm, 5.3 μm, 5.4 μm, 5.5 μm, 5.6 μm, 5.7 μm, 5.8 μm, 5.9 μm, 6.0 μm, 6.1 μm, 6.2 μm, 6.3 μm, 6.4 μm, 6.5 μm, 6.6 μm, 6.7 μm, 6.8 μm, 6.9 μm, 7.0 μm, 7.1 μm, 7.2 μm, 7.3 μm, 7.4 μm, 7.5 μm, 7.6 μm, 7.7 μm, 7.8 μm, 7.9 μm, or 8.0 μm. In another embodiment, the pharmaceutical composition has a particle size distribution by volume such that 50% of the cabotegravir particles (X50) have a particle diameter smaller than or equal to 4 μm (i.e., X50 is 4 μm). In another embodiment, the pharmaceutical composition has a particle size distribution by volume such that 50% of the cabotegravir particles (X50) have a particle diameter smaller than or equal to 6 μm (i.e., X50 is 6 μm).

In an embodiment, the pharmaceutical composition has a particle size distribution by volume such that 10% of the cabotegravir particles have a particle diameter smaller than or equal to 4 μm (i.e., X10 is 4 μm). In an embodiment, cabotegravir particles of the pharmaceutical composition have an X10 value greater than or equal to 0.5 μm and less than or equal to 4 μm (i.e., 0.5 μm≤X10≤4 μm). In an embodiment, the pharmaceutical composition has a particle size distribution by volume such that 10% of the cabotegravir particles have a particle diameter less than or equal to 3.5 μm. In an embodiment, cabotegravir particles of the pharmaceutical composition have an X10 value greater than or equal to 1 μm and less than or equal to 3.5 μm (i.e., 1 μm≤X10≤3.5 μm). In an embodiment, the pharmaceutical composition has a particle size distribution by volume such that 10% of the cabotegravir particles have a particle diameter less than or equal to 3 μm. In an embodiment, cabotegravir particles of the pharmaceutical composition have an X10 value greater than or equal to 1.5 μm and less than or equal to 3 μm (i.e., 1.5 μm≤X10≤3 μm).

In an embodiment, the pharmaceutical composition has a particle size distribution by volume such that 10% of the cabotegravir particles (X10) have a particle diameter smaller than or equal to 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm, 3.1 μm, 3.2 μm, 3.3 μm, 3.4 μm, 3.5 μm, 3.6 μm, 3.7 μm, 3.8 μm, 3.9 μm, or 4.0 μm. In another embodiment, the pharmaceutical composition has a particle size distribution by volume such that 10% of the cabotegravir particles (X10) have a particle diameter smaller than or equal to 1.7 μm (i.e., X10 is 1.7 μm). In another embodiment, the pharmaceutical composition has a particle size distribution by volume such that 10% of the cabotegravir particles (X10) have a particle diameter smaller than or equal to 2.2 μm (i.e., X10 is 2.2 μm). In another embodiment, the pharmaceutical composition has a particle size distribution by volume such that 10% of the cabotegravir particles (X10) have a particle diameter smaller than or equal to 2.6 μm (i.e., X10 is 2.6 μm).

In an embodiment, all X90, X50, and X10 values described herein are by volume and determined by laser diffraction method. The laser diffraction method is sensitive to the volume of a particle and provides a volume-average particle size, which is equivalent to the weight-average particle size if the density is constant. It will be apparent to those skilled in the art that the results of the particle size distribution determination by one technique can be correlated with that from another technique, for example on an empirical basis by routine experimentation. Alternatively, particle size distribution can be determined by microscopy, in particular electron microscopy or scanning electron microscopy.

Lyophilization comprises at least a freezing step and a sublimation step. Lyophilization may be used in the manufacturing of pharmaceutical products and intermediates thereof. During freezing, the material is cooled to a temperature wherein the solid, liquid, and gas phases of the material may exist. Active pharmaceutical product ingredients (APIs) may be lyophilized to achieve chemical and physical stability allowing room temperature storage. This is different from a conventional method that evaporates water using heat. Advantages of lyophilization may include, but are not limited to, enhanced stability of a dry powder, the removal of water without excessive heating of the product, and enhanced product stability in a dry state.

In an exemplary method of preparing a lyophilized formulation of the invention, micronized cabotegravir free acid is packaged in antistatic linear low-density polyethylene bags. The packaged cabotegravir is enclosed in a corrugated plastic box and gamma irradiated as a bioburden reduction step and referred to as gamma irradiated cabotegravir. Gamma irradiated cabotegravir is dispersed in a filtered aqueous vehicle comprising a stabilizer (e.g., sodium CMC), a tonicity agent (e.g., mannitol), and a wetting agent (e.g., PS80). The resulting suspension is filled into washed, sterilized/depyrogenated 10 mL Type I clear glass vials. Container materials are then processed: vials are depyrogenated by dry heat, and stoppers and overseals are sterilized by steam. Product vials are lyophilized, backflushed with nitrogen, sealed with halobutyl rubber stoppers and secured by an aluminum overseal. The sealed vials are terminally sterilized by gamma irradiation.

In an embodiment, the lyophilized pharmaceutical composition is a suspension. The lyophilized pharmaceutical composition advantageously suspends when reconstituted in an aqueous or non-aqueous solution, that is, all or substantially all, such as at least 90 percent, at least 95 percent, at least 96 percent, at least 97 percent, at least 98 percent, at least 99 percent, at least 99.5 percent or 100 percent of the lyophilized pharmaceutical composition is suspended when reconstituted.

Reconstitution can be assessed visually with the naked eye. The lyophilized matter is deemed reconstituted when a homogeneous suspension is observed. In particular, a suspension with a cloudy appearance is considered suitably reconstituted.

It will be apparent to those skilled in the art that pharmaceutical compositions described herein may be reconstituted in an aqueous or non-aqueous solution to a desired concentration. For example, the pharmaceutical composition described below in Examples 1-3 may be reconstituted in 1.7 mL water to achieve a cabotegravir concentration of 400 mg/mL. Similarly, the same pharmaceutical composition described below in Examples 1-3 may be reconstituted in 1.1 mL water to achieve a cabotegravir concentration of 533 mg/mL.

In an embodiment, the present disclosure provides a lyophilized pharmaceutical composition comprising cabotegravir, wherein cabotegravir is present in the form of particles having a mass median diameter (X50) of between (and including) 2.5 μm and 10 μm; a wetting agent; a stabilizer; and a tonicity adjuster; wherein the lyophilized pharmaceutical composition, when reconstituted in an aqueous solution, has a reconstitution time of 15 minutes or less, 10 minutes or less, or 5 minutes or less. In another embodiment, the present disclosure provides a lyophilized pharmaceutical composition comprising cabotegravir, wherein cabotegravir is present in the form of particles having a mass median diameter (X50) of between (and including) 2.5 μm and 10 μm; PS80; sodium CMC; and mannitol; wherein the lyophilized pharmaceutical composition, when reconstituted in an aqueous or non-aqueous solution, has a reconstitution time of 15 minutes or less, 10 minutes or less, or 5 minutes or less.

In an embodiment, the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment, the pharmaceutical composition comprises 800 mg cabotegravir (X50=6 μm); 51.8 mg mannitol; 5.9 mg PS80; and 7.4 mg sodium CMC. In an embodiment, the pharmaceutical composition comprises 800 mg cabotegravir (X10=2.2 μm); 51.8 mg mannitol; 5.9 mg PS80; and 7.4 mg sodium CMC. In an embodiment, the pharmaceutical composition comprises 800 mg cabotegravir (X90=17 μm); 51.8 mg mannitol; 5.9 mg PS80; and 7.4 mg sodium CMC. In an embodiment, the pharmaceutical composition comprises 800 mg cabotegravir (X10=2.2 μm; X50=6 μm; X90=17 μm); 51.8 mg mannitol; 5.9 mg PS80; and 7.4 mg sodium CMC.

In an embodiment, the pharmaceutical composition comprises 800 mg cabotegravir (X50=6 μm); 70.0 mg mannitol; 8.0 mg PS80; and 10.0 mg sodium CMC. In an embodiment, the pharmaceutical composition comprises 800 mg (X10=2.6 μm); 70.0 mg mannitol; 8.0 mg PS80; and 10.0 mg sodium CMC. In an embodiment, the pharmaceutical composition comprises 800 mg cabotegravir (X90=14 μm); 70.0 mg mannitol; 8.0 mg PS80; and 10.0 mg sodium CMC. In an embodiment, the pharmaceutical composition comprises 800 mg cabotegravir (X10=2.6 μm; X50=6 μm; X90=14 μm); 70.0 mg mannitol; 8.0 mg PS80; and 10.0 mg sodium CMC.

In an embodiment, the pharmaceutical composition comprises 800 mg cabotegravir (X50=4 μm); 70.0 mg mannitol; 8.0 mg PS80; and 10.0 mg sodium CMC. In an embodiment, the pharmaceutical composition comprises 800 mg cabotegravir (X10=1.7 μm); 70.0 mg mannitol; 8.0 mg PS80; and 10.0 mg sodium CMC. In an embodiment, the pharmaceutical composition comprises 800 mg cabotegravir (X90=9 μm); 70.0 mg mannitol; 8.0 mg PS80; and 10.0 mg sodium CMC. In an embodiment, the pharmaceutical composition comprises 800 mg cabotegravir (X10=1.7 μm; X50=4 μm; X90=9 μm); 70.0 mg mannitol; 8.0 mg PS80; and 10.0 mg sodium CMC.

In another embodiment, the pharmaceutical composition comprises cabotegravir, wherein cabotegravir is present in the form of particles having an X50 value greater than or equal to 3.5 μm and less than or equal to 8 μm; PS80; sodium CMC; and mannitol; wherein a weight ratio of cabotegravir: PS80:sodium CMC:mannitol is about 100:1:1.25:8.75.

In another embodiment, the pharmaceutical composition comprises cabotegravir, wherein cabotegravir is present in the form of particles having an X50 value greater than or equal 3.5 μm and less than or equal to 8 μm; PS80; sodium CMC; and mannitol; wherein a weight ratio of cabotegravir: PS80: sodium CMC:mannitol is about 400:3:3.7:25.9.

In another embodiment, the pharmaceutical composition, after lyophilization and reconstitution, is as described in Tables 1a-1 or 1a-2:

TABLE 1a-1

| Ingredient | Concentration (mg/mL) |
|---|---|
| Cabotegravir | 340-500 (X10 = 1.4-3.3 μm; X50 = |
| | 3.4-7.5 μm; X90 = 7.6-21.3 μm) |
| Polysorbate 80 | 2.5-5.0 |
| Sodium CMC | 3.1-6.3 |
| Mannitol | 22-44 |

TABLE 1a-2

| Ingredient | Concentration (mg/mL) |
|---|---|
| Cabotegravir | 340-550 (X10 = 1.4-3.3 μm; X50 = |
| | 3.4-7.5 μm; X90 = 7.6-21.3 μm) |
| Polysorbate 80 | 2.5-5.5 |
| Sodium CMC | 3.1-6.9 |
| Mannitol | 22-50 |

In another embodiment, the pharmaceutical composition, after lyophilization and reconstitution, is as described in Table 1b:

TABLE 1b

| Ingredient | Concentration (mg/mL) |
|---|---|
| Cabotegravir | 450-667 (X10 = 1.4-3.3 μm; X50 = |
| | 3.4-7.5 μm; X90 = 7.6-21.3 μm) |
| Polysorbate 80 | 2.5-5 |
| Sodium CMC | 3.1-6.9 |
| Mannitol | 22-50 |

In another embodiment, the pharmaceutical composition, after lyophilization and reconstitution with 1.7 or 1.1 mL water, is as described in Table 1c:

TABLE 1c

| | Reconstituted with | Reconstituted with |
|---|---|---|
| | 1.7 mL water | 1.1 mL water |
| Ingredient | Concentration (mg/mL) | |
| Cabotegravir | 400 (X10 = 2.2 μm; X50 = | 533 (X10 = 2.2 μm; X50 = |
| | 6 μm; X90 = 17 μm) | 6 μm; X90 = 17 μm) |
| Polysorbate 80 | 3.0 | 4.0 |
| Sodium CMC | 3.7 | 4.9 |
| Mannitol | 25.9 | 34.5 |

In another embodiment, the pharmaceutical composition, after lyophilization and reconstitution with 1.7 or 1.1 mL water, is as described in Table 1d:

TABLE 1d

| | Reconstituted with | Reconstituted with |
|---|---|---|
| | 1.7 mL water | 1.1 mL water |
| Ingredient | Concentration (mg/mL) | |
| Cabotegravir | 400 (X10 = 2.6 μm; X50 = | 533 (X10 = 2.6 μm; X50 = |
| | 6 μm; X90 = 14 μm) | 6 μm; X90 = 14 μm) |
| Polysorbate 80 | 4.0 | 5.3 |
| Sodium CMC | 5.0 | 6.7 |
| Mannitol | 35.0 | 46.6 |

In another embodiment, the pharmaceutical composition, after lyophilization and reconstitution with 1.7 or 1.1 mL water, is as described in Table 1e:

TABLE 1e

| | Reconstituted with | Reconstituted with |
|---|---|---|
| | 1.7 mL water | 1.1 mL water |
| Ingredient | Concentration (mg/mL) | |
| Cabotegravir | 400 (X10 = 1.7 μm; X50 = | 533 (X10 = 1.7 μm; X50 = |
| | 4 μm; X90 = 9 μm) | 4 μm; X90 = 9 μm) |
| Polysorbate 80 | 4.0 | 5.3 |
| Sodium CMC | 5.0 | 6.7 |
| Mannitol | 35.0 | 46.6 |

In an embodiment the pharmaceutical composition of the invention has a pH of about 4 or greater. A pH of about 4 or more reduces pain to the patient if the composition is administered via injection. In an embodiment, the pharmaceutical composition of the invention has a pH of about 6.5. In an alternative embodiment, the pharmaceutical composition has a pH in the range of about 4 to about 8. In an alternative embodiment, the pharmaceutical composition has a pH in the range of about 5 to about 7. In an alternative embodiment, the pharmaceutical composition has a pH in the range of about 6 to about 7.

In another embodiment, the pharmaceutical composition comprises about 200 mg/ml of cabotegravir. In an embodiment, the pharmaceutical composition comprises about 200 mg/ml of cabotegravir and at least one pharmaceutically acceptable excipient. In an embodiment, the pharmaceutical composition comprises 200 mg/mL of cabotegravir. In an embodiment, the pharmaceutical composition comprises 200 mg/mL of cabotegravir and at least one pharmaceutically acceptable excipient. In an embodiment the pharmaceutical composition is an injectable suspension. In an embodiment the pharmaceutical composition comprises about 600 mg of cabotegravir. In an embodiment, the pharmaceutical composition comprises about 600 mg of cabotegravir and at least one pharmaceutically acceptable excipient. In another embodiment of the invention, the pharmaceutical composition comprises 600 mg of cabotegravir. In another embodiment, the pharmaceutical composition comprises 600 mg of cabotegravir and at least one pharmaceutically acceptable excipient.

In an embodiment the pharmaceutical composition further comprises polyethylene glycol (PEG), which is a pharmaceutically acceptable excipient. In an embodiment the PEG is PEG 3350. In an embodiment, the pharmaceutical composition further comprises polysorbate 20, which is a pharmaceutically acceptable excipient. In a further embodiment, the pharmaceutical composition further comprises mannitol, which is a pharmaceutically acceptable excipient. In an embodiment, the pharmaceutical composition comprises 600 mg of cabotegravir, PEG 3350, polysorbate 20, and mannitol. In an embodiment, the pharmaceutical composition is as described in WO2012/037320 which is incorporated herein by reference.

In an embodiment the pharmaceutical composition is suitable for use as injectable. In an embodiment, the pharmaceutical composition is an injectable suspension. In an embodiment, the pharmaceutical composition is an injectable suspension suitable for gluteal administration.

In an embodiment the pharmaceutical composition is APRETUDE®. APRETUDE®, also referred to herein as Apretude, is an approved extended release cabotegravir injectable suspension used to prevent HIV infection. Apretude comprises 3 mL of the composition shown in Table 2.

TABLE 2

| Ingredient | Concentration |
|---|---|
| Cabotegravir | 200 mg/mL |
| Mannitol | 35 mg/mL |
| Polyethylene glycol (PEG) 3350 | 20 mg/mL |
| Polysorbate 20 | 20 mg/mL |

### Coadministration

In an embodiment, the method is a method of treating HIV and the pharmaceutical composition is administered to the patient in combination with a second anti-HIV agent. In this embodiment the second anti-HIV agent may be any known anti-HIV agent. In an embodiment, the second anti-HIV agent is a non-nucleoside reverse transcriptase inhibitor (NNRTI), a nucleoside reverse transcriptase inhibitor (NRTI) or a capsid inhibitor or a broadly neutralising antibody.

### Exemplary Embodiments

In an embodiment, the cabotegravir provides a Ctau of at least 1 μg/mL, and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment, the cabotegravir provides, a Ctau of at least 1.05 μg/mL and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment the method comprises first administering a loading dose of the pharmaceutical composition to the human; 2 to 6 weeks after administering the loading dose, administering a maintenance dose of the pharmaceutical composition to the human;

and then administering the maintenance dose every 15 to 24 weeks thereafter wherein the cabotegravir provides, a Ctau of at least 1.05 μg/mL and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment the method comprises first administering a loading dose of the pharmaceutical composition to the human wherein the first loading dose comprises at least 1600 mg of cabotegravir; 2 to 6 weeks after administering the loading dose, administering a maintenance dose of the pharmaceutical composition to the human; and then administering the maintenance dose every 15 to 24 weeks thereafter, wherein the maintenance dose comprises at least 1000 mg of cabotegravir and wherein the cabotegravir provides, a Ctau of at least 1.05 μg/mL and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment the method comprises first administering a loading dose of the pharmaceutical composition to the human wherein the first loading dose comprises at least 1600 mg of cabotegravir; 1 month i.e. 3 to 5 weeks after administering the loading dose, administering a maintenance dose of the pharmaceutical composition to the human; and then administering the maintenance dose every 4 months i.e. 6 to 8 weeks thereafter, wherein the maintenance dose comprises at least 1000 mg of cabotegravir and wherein the cabotegravir provides, a Ctau of at least 1.05 μg/mL and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment the method comprises first administering a loading dose of the pharmaceutical composition to the human wherein the first loading dose comprises at least 1600 mg of cabotegravir; 2 to 6 weeks after administering the loading dose, administering a maintenance dose of the pharmaceutical composition to the human; and then administering the maintenance dose every 15 to 24 weeks thereafter, wherein the maintenance dose comprises at least 1000 mg of cabotegravir and wherein the cabotegravir provides, a Ctau of at least 1.05 μg/mL and the pharmaceutical composition comprises cabotegravir in a concentration of 533 mg/mL, mannitol, PS80, and sodium CMC; wherein cabotegravir is present in the form of particles having a mass median diameter (X50) of between (and including) 2.5 μm and 10 μm.

In an embodiment the method comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter wherein the pharmaceutical composition is as described in Table 1a-1.

In an embodiment the method comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter wherein the pharmaceutical composition is as described in Table 1a-2.

In an embodiment the method comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter wherein the pharmaceutical composition is as described in Table 1a-1 and wherein the cabotegravir provides a Ctau of at least 1.05 μg/mL.

In an embodiment the method comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter wherein the pharmaceutical composition is as described in Table 1a-2 and wherein the cabotegravir provide a Ctau of at least 1.05 μg/mL.

In an embodiment the loading dose of the pharmaceutical composition comprises 1600 to 4000 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises 1000 to 1800 mg of cabotegravir.

In an embodiment the loading dose of the pharmaceutical composition comprises 3000 to 4000 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises 1400 to 1800 mg of cabotegravir.

In an embodiment the loading dose of the pharmaceutical composition comprises 3000 to 4000 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises 1400 to 1800 mg of cabotegravir wherein the pharmaceutical composition comprises cabotegravir, mannitol, PS80, and sodium CMC; wherein cabotegravir is present in the form of particles having a mass median diameter (X50) of between (and including) 2.5 μm and 10 μm and the pharmaceutical composition has a cabotegravir concentration of about 533 mg/mL.

In an embodiment the loading dose of the pharmaceutical composition comprises 3000 to 4000 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises 1400 to 1800 mg of cabotegravir wherein the pharmaceutical composition comprises cabotegravir, mannitol, PS80, and sodium CMC; wherein cabotegravir is present in the form of particles having a mass median diameter (X50) of between (and including) 2.5 μm and 10 μm and the pharmaceutical composition has a cabotegravir concentration of about 400 mg/mL.

In an embodiment the loading dose of the pharmaceutical composition comprises about 3200 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises about 1600 mg of cabotegravir wherein the pharmaceutical composition comprises cabotegravir, mannitol, PS80, and sodium CMC; wherein cabotegravir is present in the form of particles having a mass median diameter (X50) of between (and including) 2.5 μm and 10 μm and the pharmaceutical composition has a cabotegravir concentration of about 533 mg/mL.

In an embodiment the loading dose of the pharmaceutical composition comprises about 3200 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises about 1600 mg of cabotegravir wherein the pharmaceutical composition comprises cabotegravir, mannitol, PS80, and sodium CMC; wherein cabotegravir is present in the form of particles having a mass median diameter (X50) of between (and including) 2.5 μm and 10 μm and the pharmaceutical composition has a cabotegravir concentration of about 400 mg/mL.

### Use in the Treatment or Prevention of HIV

In a third aspect, the present invention provides a pharmaceutical composition for use in the treatment of HIV, wherein the pharmaceutical composition comprises cabotegravir and the treatment comprises administering the pharmaceutical composition to a human once every 15 to 24 weeks.

In a fourth aspect, the present invention provides a pharmaceutical composition for use in the prevention of HIV, wherein the pharmaceutical composition comprises cabotegravir and the prevention comprises said prevention comprising administering the pharmaceutical composition to a human once every 15 to 24 weeks.

The embodiments discussed below apply equally to the third and fourth aspects.

Cabotegravir is as discussed herein above.

In an embodiment of the invention, cabotegravir is present in the pharmaceutical composition as the free acid.

The dose of cabotegravir administered, which is the amount of cabotegravir in the composition for use in the invention, may be selected such that: the blood plasma concentration of cabotegravir in the human is kept above a trough plasma concentration (Ctau); or the blood plasma concentration of cabotegravir in the human is maintained at or above the Ctau of the approved 200 mg/mL cabotegravir regimen i.e. in CABENUVA® or APRETUDE®.

Trough plasma level (Ctau) refers to trough plasma concentration i.e. the concentration reached immediately before the next dose is administered. The Ctau value represents the lowest blood plasma level. The Ctau can be measured in any suitable way.

The present inventors have found that, to maintain an effective dosing regimen for treatment or prevention of HIV, the dose of cabotegravir, which is the amount of cabotegravir in the pharmaceutical composition for use in the invention, should maintain Ctau at a level higher than the 10th percentile of Ctau observed in the Phase 3 studies 201738 (HPTN 083) for people assigned male at birth (1.05 μg/mL) and 201739 (HPTN 084) for people assigned female at birth (1.39 μg/mL). The 10th percentile of Ctau observed in the Phase 3 studies mentioned are the sex-specific PrEP benchmarks which were achieved in at least 90% of participants or the dose of cabotegravir needs to maintain the median and 10th percentile of Ctau higher than those from the approved 200 mg/ml intramuscular regimen of cabotegravir in both people who were assigned male at birth and people who were assigned female at birth.

In an embodiment, the dose of cabotegravir administered is selected such that the Ctau is higher than the Ctau observed in the approved 200 mg/mL intramuscular regimen of cabotegravir in both people who were assigned male at birth and people who were assigned female at birth. In an embodiment, the dose of cabotegravir administered is selected such that the Ctau is higher than the Ctau observed in the dosing regimen of APRETUDE®. In an embodiment, the dose of cabotegravir administered is selected such that the Ctau is higher than the Ctau observed in the 10th percentile observed in the dosing regimen of APRETUDE®.

In an embodiment the dose of cabotegravir administered is selected such that the Ctau is at least 1 μg/mL i.e. the cabotegravir provides a Ctau of at least about 1 μg/mL. In another embodiment the cabotegravir provides a Ctau of at least 1.05 μg/mL. In another embodiment the human was assigned female at birth and the cabotegravir provides a Ctau of at least about 1.35 μg/mL. In another embodiment the human was assigned female at birth and the cabotegravir provides a Ctau of at least about 1.39 μg/mL. In another embodiment the human was assigned female at birth and the cabotegravir provides a Ctau of at least 1.39 μg/mL. In an embodiment the human was assigned male at birth and the cabotegravir provides a Ctau of at least about 1.05 μg/mL. In another embodiment the human was assigned male at birth and the cabotegravir provides a Ctau of at least 1.05 μg/mL. In an embodiment, the human was assigned female at birth and the cabotegravir provides a Ctau of at least about 1.39 μg/mL; or the human was assigned male at birth and the cabotegravir provides a Ctau of at least about 1.05 μg/mL.

In an embodiment, the Ctau levels mentioned above are achieved in at least 90% of humans being administered the dosing regimen.

The blood plasma levels of cabotegravir in a human may be kept above these Ctau levels because at lower levels the drug may no longer be effective, thereby increasing the risk of transmission of HIV infection, and may be suboptimal for treatment of HIV infected subjects. Plasma levels of cabotegravir may be kept at higher levels to avoid the development of HIV mutations, while maintaining a safety margin.

In an embodiment the use comprises first administering a loading dose of the pharmaceutical composition to the human; 15 to 24 weeks after administering the loading dose, administering a maintenance dose of the pharmaceutical composition to the human; and then administering the maintenance dose every 15 to 24 weeks thereafter.

In an embodiment, the human is screened for HIV-1 infection immediately prior to receiving the loading dose or oral lead in. In an embodiment, the pharmaceutical composition is for use in the prevention of HIV and human is screened for HIV-1 infection immediately prior to receiving the loading dose or oral lead in.

The loading dose is a single dose of the pharmaceutical composition received at the initiation of use. The maintenance dose is then administered to the human every 15 to 24 weeks thereafter.

The pharmaceutical composition used for the loading dose and the maintenance dose are the same composition, but they may comprise different concentrations or amounts of cabotegravir. In an embodiment of the invention, the pharmaceutical composition used for the loading dose and the maintenance dose are the same concentration, but the loading dose is administered in a higher volume. In an embodiment the loading dose and the maintenance dose are the same dose (for example, the loading dose and the maintenance dose are the same volume and each comprises the same pharmaceutical composition, with the same concentration and same amount of cabotegravir). In an embodiment the loading dose and maintenance dose are different doses (for example, the loading dose and the maintenance dose are different volumes of the same pharmaceutical composition with the same concentration of cabotegravir, or the loading dose and the maintenance dose are different volumes of different pharmaceutical compositions with different concentrations of cabotegravir). In an embodiment, the loading dose of cabotegravir is higher than the maintenance dose of cabotegravir (for example, the loading dose and the maintenance dose each comprise the same concentration of cabotegravir wherein the volume of the loading dose is greater than the volume of the maintenance dose, or the loading dose and the maintenance dose comprises different concentrations of cabotegravir wherein the volume of the loading dose is greater than the volume of the maintenance dose).

In another embodiment, the pharmaceutical composition used for the loading dose is a different pharmaceutical composition as the pharmaceutical composition used for the maintenance dose (for example, the pharmaceutical composition used for the loading dose has different excipient(s) from the pharmaceutical composition used for the maintenance dose).

In yet another embodiment, a loading dose comprises two different pharmaceutical compositions.

### Method of Administration

The pharmaceutical compositions described in the present invention are suitable for injection. In an embodiment the pharmaceutical composition is administered intramuscularly or subcutaneously via injection. In an embodiment the pharmaceutical composition is administered subcutaneously. In an embodiment the pharmaceutical composition is administered intramuscularly. In an embodiment, the pharmaceutical composition is administered in a gluteus muscle of the human. In a specific embodiment, the pharmaceutical composition is administered at the ventrogluteal or dorsogluteal site. In an embodiment the pharmaceutical composition is administered in the gluteus medius, the gluteus maximus, or both the gluteus medius and the gluteus maximus. In an embodiment the pharmaceutical composition is administered in the gluteus medius. In an embodiment the pharmaceutical composition is administered in the gluteus maximus.

### Loading Dose

In an embodiment the loading dose of the pharmaceutical composition comprises at least 600 mg of cabotegravir. In an embodiment the loading dose of the pharmaceutical composition comprises at least 1200 mg of cabotegravir. In an embodiment the loading dose of the pharmaceutical composition comprises at least 1400 mg of cabotegravir. In an embodiment the loading dose of the pharmaceutical composition comprises at least 1500 mg of cabotegravir. In another embodiment the loading dose of the pharmaceutical composition comprises at least 1600 mg of cabotegravir. In another embodiment the loading dose of the pharmaceutical composition comprises at least 1800 mg of cabotegravir. In another embodiment the loading dose of the pharmaceutical composition comprises at least 2000 mg of cabotegravir. In another embodiment the loading dose of the pharmaceutical composition comprises at least 2200 mg of cabotegravir. In another embodiment the loading dose of the pharmaceutical composition comprises at least 2400 mg of cabotegravir. In another embodiment the loading dose of the pharmaceutical composition comprises at least 2600 mg of cabotegravir. In another embodiment the loading dose of the pharmaceutical composition comprises at least 2800 mg of cabotegravir. In another embodiment the loading dose of the pharmaceutical composition comprises at least 3000 mg of cabotegravir. In an embodiment, the loading dose comprises about 600 mg to about 4000 mg of cabotegravir. In an embodiment, the loading dose comprises about 1200 mg to about 4000 mg of cabotegravir. In an embodiment the loading dose of the pharmaceutical composition comprises about 2000 mg to about 3500 mg of cabotegravir. In an alternative embodiment, the loading dose of the pharmaceutical composition comprises 3000 mg to about 3500 mg of cabotegravir. In an alternative embodiment, the loading dose of the pharmaceutical composition comprises 3100 mg to about 3300 mg of cabotegravir. In an alternative embodiment, the loading dose of the pharmaceutical composition comprises 3150 mg to about 3250 mg of cabotegravir. In an embodiment, the loading dose of the pharmaceutical composition comprises about 600 mg of cabotegravir. In an embodiment, the loading dose of the pharmaceutical composition comprises about 1600 mg of cabotegravir. In an embodiment, the loading dose of the pharmaceutical composition comprises about 2000 mg of cabotegravir. In an embodiment, the loading dose of the pharmaceutical composition comprises about 2132 mg of cabotegravir. In an embodiment, the loading dose of the pharmaceutical composition comprises about 2200 mg of cabotegravir. In an embodiment, the loading dose of the pharmaceutical composition comprises about 2400 mg of cabotegravir. In an embodiment, the loading dose of the pharmaceutical composition comprises about 3200 mg of cabotegravir. In an embodiment, the loading dose of the pharmaceutical composition comprises 3200 mg of cabotegravir. In an embodiment, the loading dose of the pharmaceutical composition comprises 600 mg of cabotegravir. In an embodiment, the loading dose of the pharmaceutical composition comprises 1600 mg of cabotegravir. In an embodiment, the loading dose of the pharmaceutical composition comprises 2200 mg of cabotegravir. In an embodiment, the loading dose of the pharmaceutical composition comprises 2400 mg of cabotegravir. In an embodiment, the loading dose of the pharmaceutical composition comprises 3200 mg of cabotegravir.

The loading dose may be administered in one injection or in two separate injections. In embodiments where the loading dose is administered in two separate injections, these injections are administered sequentially. In an embodiment where the loading dose is administered in two separate injections, the two separate injections are administered in the same site or in different sites. In an embodiment where the loading dose is administered in two separate injections, the two separate injections are administered sequentially in the same site. In another embodiment where the loading dose is administered in two separate injections, the two separate injections are administered sequentially in sites at least 2 cm apart (for example, one injection in left gluteus maximus and one injection in right gluteus maximus, or one injection in left gluteus medius and the other injection in right gluteus medius). In an embodiment, each of the two separate injections comprises the same amount of cabotegravir. In an embodiment, each of the two separate injections comprises different amounts of cabotegravir (for example, one injection comprises about 600 mg of cabotegravir and the other injection comprises about 1600 mg of cabotegravir).

In an embodiment where the loading dose is administered in two separate injections, the injections each contain the same pharmaceutical composition or different pharmaceutical compositions. In an embodiment where the loading dose is administered in two separate injections, the injections each contain the same pharmaceutical composition. In an embodiment where the loading dose is administered in two separate injections, the injections contain different pharmaceutical compositions.

In an embodiment, the loading dose of the pharmaceutical composition comprises 600 mg of cabotegravir wherein the loading dose is administered in one 2 mL injection.

In an embodiment, the loading dose of the pharmaceutical composition comprises 1600 mg of cabotegravir wherein the loading dose is administered in one 3 mL injection.

In an embodiment, the loading dose of the pharmaceutical composition comprises 2200 mg of cabotegravir wherein the loading dose is administered in two separate injections, wherein one injection comprises about 600 mg of cabotegravir and the other injection comprises about 1600 mg of cabotegravir.

In an embodiment, the loading dose of the pharmaceutical composition comprises 2400 mg of cabotegravir wherein the loading dose is administered in two separate injections, each comprising about 1200 mg of cabotegravir.

In an embodiment, the loading dose of the pharmaceutical composition comprises 3200 mg of cabotegravir wherein the loading dose is administered in two separate injections, each comprising about 1600 mg of cabotegravir.

In an embodiment, the loading dose is administered as a 1 mL injection. In an embodiment, the loading dose is administered as a 2 mL injection. In an embodiment the loading dose is administered as a 2.25 mL injection. In an embodiment, the loading dose is administered as a 3 mL injection.

In an embodiment, the loading dose of the pharmaceutical composition comprises 2132 mg of cabotegravir wherein the loading dose is administered in two separate 2 mL injections, each comprising about 1066 mg of cabotegravir.

In an embodiment, the loading dose of the pharmaceutical composition comprises 2200 mg of cabotegravir wherein the loading dose is administered in two separate injections, one comprising about 600 mg of cabotegravir in a 2 mL injection and the other comprising about 1600 mg cabotegravir in a 3 mL injection.

In an embodiment, the loading dose of the pharmaceutical composition comprises 2400 mg of cabotegravir wherein the loading dose is administered in two separate injections, each comprising about 1200 mg of cabotegravir in a 2.25 mL injection.

In an embodiment, the loading dose of the pharmaceutical composition comprises 3200 mg of cabotegravir wherein the loading dose is administered in two separate 3 mL injections, each comprising about 1600 mg of cabotegravir.

In another embodiment, the loading dose may refer to a first loading dose when one or more subsequent loading doses is administered at a later point in time (e.g., a second loading dose is administered 2-6 weeks after a first loading dose). Subsequent loading doses are as described in any of the preceding embodiments of a loading dose.

In an embodiment, a first loading dose is followed by a second loading dose. In another embodiment, a first loading dose is followed by a second loading dose and a third loading dose. In another embodiment, a first loading dose is followed by a second loading dose, a third loading dose, and a fourth loading dose (for example, a second loading dose is administered 2-6 weeks after a first loading dose, a third loading dose is administered 6-11 weeks after the second loading dose, and a fourth loading dose is administered 6-11 weeks after the third loading dose).

### Maintenance Dose

In an embodiment, the maintenance dose of the pharmaceutical composition comprises at least 1000 mg of cabotegravir. In another embodiment the maintenance dose of the pharmaceutical composition comprises at least 1200 mg of cabotegravir. In an embodiment, the maintenance dose of the pharmaceutical composition comprises at least 1300 mg of cabotegravir. In an embodiment, the maintenance dose of the pharmaceutical composition comprises at least 1330 mg of cabotegravir. In another embodiment the maintenance dose of the pharmaceutical composition comprises at least 1500 mg of cabotegravir. In another embodiment the maintenance dose of the pharmaceutical composition comprises at least 1600 mg of cabotegravir. In an embodiment, the maintenance dose comprises about 1000 mg to about 1800 mg of cabotegravir. In an embodiment, the maintenance dose comprises about 1200 mg to about 1800 mg of cabotegravir. In another embodiment the maintenance dose of the pharmaceutical composition comprises about 1250 mg to about 1800 mg of cabotegravir. In another embodiment the maintenance dose of the pharmaceutical composition comprises about 1300 mg to about 1800 mg of cabotegravir. In another embodiment the maintenance dose of the pharmaceutical composition comprises about 1400 mg to about 1800 mg, about 1400 to about 1700 mg, about 1450 to about 1700 mg, about 1450 to about 1650 mg, about 1500 mg to about 1650 mg, or about 1550 mg to about 1650 mg of cabotegravir. In an embodiment the maintenance dose of the pharmaceutical composition comprises about 1550 mg of cabotegravir. In an embodiment the maintenance dose of the pharmaceutical composition comprises 1550 mg of cabotegravir. In an embodiment the maintenance dose of the pharmaceutical composition comprises about 1600 mg of cabotegravir. In an embodiment the maintenance dose of the pharmaceutical composition comprises 1600 mg of cabotegravir. In an embodiment the maintenance dose of the pharmaceutical composition comprises about 1650 mg of cabotegravir. In an embodiment the maintenance dose of the pharmaceutical composition comprises 1650 mg of cabotegravir.

In an embodiment, the maintenance dose is administered as a 1 mL injection. In an embodiment, the maintenance dose is administered as a 2 mL injection. In an embodiment, the maintenance dose is administered as a 3 mL injection.

In an embodiment the maintenance dose of the pharmaceutical composition comprises about 1066 mg of cabotegravir in a 2 mL injection, about 1332 mg of cabotegravir in a 2.5 mL injection or about 1600 mg of cabotegravir in a 3 mL injection.

In an embodiment the maintenance dose of the pharmaceutical composition comprises about 1066 mg of cabotegravir in a 2 mL injection.

In an embodiment the maintenance dose of the pharmaceutical composition comprises about 1600 mg of cabotegravir in a 3 mL injection.

In an embodiment, the first maintenance dose is administered 2 to 6 weeks after administering the loading dose and then every 15 to 24 weeks thereafter.

In an embodiment, the first maintenance dose is administered 2 to 6 weeks, or 3 to 4 weeks after the loading dose. In an embodiment the first maintenance dose is administered 1 months after the loading dose i.e. 3 to 5 weeks after the loading dose. In an embodiment, the first maintenance dose is administered about 3 weeks after the loading dose, about 4 weeks after the loading dose or about 5 weeks after the loading dose. In an embodiment, the first maintenance dose is administered 3 weeks after the loading dose, 4 weeks after the loading dose or 5 weeks after the loading dose.

In another embodiment, the first maintenance dose is administered 6 to 11 weeks after administering the loading dose and then every 15 to 24 weeks thereafter.

In another embodiment, the first maintenance dose is administered 6 to 11 weeks, or 7 to 10 weeks after the loading dose. In an embodiment the first maintenance dose is administered about 2 months after the loading dose. In an embodiment the first maintenance dose is administered 2 months after the loading dose. In an embodiment, the first maintenance dose is administered about 7 weeks after the loading dose, about 8 weeks after the loading dose, about 9 weeks after the loading dose, or about 10 weeks after the loading dose. In an embodiment, the first maintenance dose is administered 7 weeks after the loading dose, 8 weeks after the loading dose, 9 weeks after the loading dose, or 10 weeks after the loading dose.

In an embodiment, the maintenance dose is administered once every 15 to 20, or every 15 to 19 weeks.

In an embodiment the maintenance dose is administered once every 4 months i.e. once every 16 to 18 weeks. In an embodiment, the maintenance dose is administered once every 16 weeks, every 17 weeks or every 18 weeks.

In an embodiment, the maintenance dose of the pharmaceutical composition comprises at least 1200 mg of cabotegravir and is administered every 4 months i.e. every 16 to 18 weeks. In an embodiment, the maintenance dose of the pharmaceutical composition comprises 1200 mg to 1800 mg of cabotegravir and is administered every 4 months i.e. every 16 to 18 weeks. In an embodiment, the maintenance dose of the pharmaceutical composition comprises about 1600 mg of cabotegravir and is administered every 4 months i.e. every 16 to 18 weeks.

In an embodiment, the use comprises first administering a loading dose of the pharmaceutical composition to the human wherein the pharmaceutical composition comprises about 1200 mg to 4000 mg of cabotegravir; 1 month or 3 to 5 weeks after administering the loading dose, administering a maintenance dose of the pharmaceutical composition comprising 1200 to 1800 mg of cabotegravir to the human; and then administering the maintenance dose every 4 months or 16 to 18 weeks thereafter.

In an embodiment, the use comprises first administering a loading dose of the pharmaceutical composition to the human wherein the pharmaceutical composition comprises about 3000 mg to 3500 mg of cabotegravir; 1 month or 3 to 5 weeks after administering the loading dose, administering a maintenance dose of the pharmaceutical composition comprising 1500 to 1700 mg of cabotegravir to the human; and then administering the maintenance dose every 4 months or 16 to 18 weeks thereafter.

In an embodiment, the use comprises first administering a loading dose of the pharmaceutical composition to the human wherein the pharmaceutical composition comprises about 3200 mg of cabotegravir administered in two injections, each comprising 1600 mg of cabotegravir; 1 months or 3 to 5 weeks after administering the loading dose, administering a maintenance dose of the pharmaceutical composition comprising about 1600 mg of cabotegravir to the human; and then administering the maintenance dose every 4 months or 16 to 18 weeks thereafter.

In an embodiment, the use comprises first administering a first loading dose of the pharmaceutical composition(s) to the human wherein the pharmaceutical composition(s) comprises about 3200 mg of cabotegravir, wherein the first loading dose is optionally administered in two separate injections, each comprising 1600 mg of cabotegravir; 4 months or 16 to 18 weeks after administering the first loading dose, administering to the human a second loading dose of the pharmaceutical composition(s) comprising about 1600 mg of cabotegravir; 4 months or 16 to 18 weeks after administering the second loading dose, administering to the human a maintenance dose of the pharmaceutical composition(s) comprising about 1600 mg of cabotegravir; and then administering to the human the maintenance dose comprising about 1600 mg of cabotegravir every 4 months or 16 to 18 weeks thereafter.

In an embodiment, the use comprises first administering a first loading dose of the pharmaceutical composition(s) to the human wherein the pharmaceutical composition(s) comprises about 1600 mg of cabotegravir; 1 month or 3 to 5 weeks after administering the first loading dose, administering a second loading dose of the pharmaceutical composition(s) comprising about 1600 mg of cabotegravir to the human; 2 months or 7 to 10 weeks after administering the second loading dose, administering to the human a third loading dose of the pharmaceutical composition(s) comprising about 1600 mg of cabotegravir; four months or 16 to 18 weeks after administering the third loading dose, administering to the human a maintenance dose of the pharmaceutical composition(s) comprising about 1600 mg of cabotegravir; and then administering to the human the maintenance dose every 4 months or 16 to 18 weeks thereafter.

In an embodiment, the use comprises first administering a first loading dose of the pharmaceutical composition(s) to the human wherein the pharmaceutical composition(s) comprises about 2400 mg of cabotegravir, wherein the first loading dose is optionally administered in two separate injections, each comprising 1200 mg of cabotegravir; 2 months or 7 to 10 weeks after administering the first loading dose, administering to the human a second loading dose of the pharmaceutical composition(s) comprising about 1600 mg of cabotegravir; 4 months or 16 to weeks after administering the second loading dose, administering to the human a maintenance dose of the pharmaceutical composition(s) comprising about 1600 mg of cabotegravir; and then administering to the human the maintenance dose comprising about 1600 mg of cabotegravir every 4 months or 16 to 18 weeks thereafter.

In an embodiment, the use comprises first administering a first loading dose of the pharmaceutical composition(s) to the human wherein the pharmaceutical composition(s) comprises about 2200 mg of cabotegravir, wherein the first loading dose is optionally administered in two separate injections (e.g., one injection of about 600 mg cabotegravir and another injection of about 1600 mg cabotegravir); 2 months or 7 to 10 weeks after administering the first loading dose, administering to the human a second loading dose of the pharmaceutical composition(s) comprising about 1600 mg of cabotegravir; 2 months or 7 to 10 weeks after administering the second loading dose, administering to the human a third loading dose of the pharmaceutical composition(s) comprising about 1600 mg of cabotegravir; four months or 16 to 18 weeks after administering the third loading dose, administering to the human a maintenance dose of the pharmaceutical composition comprising about 1600 mg of cabotegravir; and then administering to the human the maintenance dose comprising about 1600 mg of cabotegravir every 4 months or 16 to 18 weeks thereafter.

In an embodiment, the use comprises first administering a first loading dose of the pharmaceutical composition(s) to the human wherein the pharmaceutical composition(s) comprises about 600 mg of cabotegravir; 1 month or 3 to 5 weeks after administering the first loading dose, administering a second loading dose of the pharmaceutical composition(s) comprising about 1600 mg of cabotegravir to the human; 2 months or 7 to 10 weeks after administering the second loading dose, administering to the human a third loading dose of the pharmaceutical composition(s) comprising about 1600 mg of cabotegravir; 4 months or 16 to 18 weeks after administering the third loading dose, administering to the human a maintenance dose of the pharmaceutical composition(s) comprising about 1600 mg of cabotegravir; and then administering to the human the maintenance dose every 4 months or 16 to 18 weeks thereafter.

In an embodiment, the use comprises first administering a first loading dose of the pharmaceutical composition(s) to the human wherein the pharmaceutical composition(s) comprises about 600 mg of cabotegravir; 1 month or 3 to 5 weeks after administering the first loading dose, administering a second loading dose of the pharmaceutical composition(s) comprising about 600 mg of cabotegravir to the human; 2 months or 7 to 10 weeks after administering the second loading dose, administering to the human a third loading dose of the pharmaceutical composition(s) comprising about 1600 mg of cabotegravir; 2 months or 7 to 10 weeks after administering the third loading dose, administering to the human a fourth loading dose of the pharmaceutical composition(s) comprising about 1600 mg of cabotegravir; 4 months or 16 to 18 weeks after administering the fourth loading dose, administering to the human a maintenance dose of the pharmaceutical composition comprising about 1600 mg of cabotegravir; and then administering to the human the maintenance dose every 4 months or 16 to 18 weeks thereafter.

### Oral Lead-In and Oral Bridging

In an embodiment, prior to the loading dose, an oral lead-in is given to the human to assess tolerability of cabotegravir. In an embodiment, the oral lead-in is taken once daily for about 1 month i.e. at least 28 days prior to administering the loading dose. In an embodiment the oral lead-in is a tablet of cabotegravir taken once daily. In an embodiment, the tablet comprises cabotegravir sodium. In an embodiment, the tablet comprises about 30 mg of cabotegravir sodium. In an embodiment, the tablet comprises 30 mg of cabotegravir (equivalent to 31.62 mg of cabotegravir sodium). In an embodiment, the loading dose is administered on the last day of oral lead-in. In an embodiment, the loading dose is administered within 3 days of stopping the oral lead in.

In an embodiment, the maintenance dose is discontinued and replaced with oral tablets. In an embodiment, the oral tablet is taken once daily. In an embodiment the oral tablet is a tablet of cabotegravir taken once daily. In an embodiment, the tablet comprises cabotegravir sodium. In an embodiment, the tablet comprises about 30 mg of cabotegravir sodium. In an embodiment, the tablet comprises 30 mg of cabotegravir (equivalent to 31.62 mg of cabotegravir sodium). In an embodiment, the human restarts the maintenance dose after taking daily oral tablets for a maximum of 4 months or a maximum of 16 to 18 weeks. In this embodiment, the maintenance dose is discontinued and replaced with oral tablets of cabotegravir taken daily for up to 4 months or up to 16 to 18 weeks and then the maintenance dose is reinitiated and taken once every 15 to 20 weeks thereafter.

In a separate embodiment, the human is taking daily oral tablets for more than 4 months or more than 16 to 18 weeks, in this embodiment the loading dose is taken before the maintenance dose is reinitiated. In this embodiment, the maintenance dose is discontinued and replaced with oral tablets of cabotegravir taken daily for at least 4 months or at least 16 to 18 weeks and then the loading dose is administered to the human; 2 to 6 weeks after administering the loading dose, administering the maintenance dose of the pharmaceutical composition to the human; and then administering the maintenance dose every 15 to 24 weeks thereafter.

### Pharmaceutical Compositions

The pharmaceutical composition is as described hereinabove for the first and second aspect.

### Coadministration

In an embodiment, the use is a use in the treatment of HIV and the pharmaceutical composition is administered to the patient in combination with a second anti-HIV agent. In this embodiment the second anti-HIV agent may be any known anti-HIV agent. In an embodiment, the second anti-HIV agent is a non-nucleoside reverse transcriptase inhibitor (NNRTI), a nucleoside reverse transcriptase inhibitor (NRTI) or a capsid inhibitor or a broadly neutralising antibody.

### Exemplary Embodiments

In an embodiment, the cabotegravir provides a Ctau of at least 1 μg/mL, and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment, the cabotegravir provides, a Ctau of at least 1.05 μg/mL and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment the use comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter wherein the cabotegravir provides, a Ctau of at least 1.05 μg/mL and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment the use comprises first administering a loading dose of the pharmaceutical composition to the human wherein the first loading dose comprises at least 1600 mg of cabotegravir; 2 to 6 weeks after administering the loading dose, administering a maintenance dose of the pharmaceutical composition to the human; and then administering the maintenance dose every 15 to 24 weeks thereafter, wherein the maintenance dose comprises at least 1200 mg of cabotegravir and wherein the cabotegravir provides, a Ctau of at least 1.05 μg/mL and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment the use comprises first administering a loading dose of the pharmaceutical composition to the human wherein the first loading dose comprises at least 1600 mg of cabotegravir; 2 to 6 weeks after administering the loading dose, administering a maintenance dose of the pharmaceutical composition to the human; and then administering the maintenance dose every 15 to 24 weeks thereafter, wherein the maintenance dose comprises at least 1000 mg of cabotegravir and wherein the cabotegravir provides, a Ctau of at least 1.05 μg/mL and the pharmaceutical composition comprises cabotegravir in a concentration of 533 mg/mL, mannitol, PS80, and sodium CMC; wherein cabotegravir is present in the form of particles having a mass median diameter (X50) of between (and including) 2.5 μm and 10 μm.

In an embodiment the use comprises first administering a loading dose of the pharmaceutical composition to the human wherein the first loading dose comprises at least 1600 mg of cabotegravir; 1 month i.e. 3 to 5 weeks after administering the loading dose, administering a maintenance dose of the pharmaceutical composition to the human; and then administering the maintenance dose every 4 months i.e. 6 to 8 weeks thereafter, wherein the maintenance dose comprises at least 1200 mg of cabotegravir and wherein the cabotegravir provides, a Ctau of at least 1.05 μg/mL and the pharmaceutical composition comprises cabotegravir, mannitol, PS80, and sodium CMC; wherein cabotegravir is present in the form of particles having a mass median diameter (X50) of between (and including) 2.5 μm and 10 μm.

In an embodiment the use comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter wherein the pharmaceutical composition is as described in Table 1a-1.

In an embodiment the use comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter wherein the pharmaceutical composition is as described in Table 1a-2.

In an embodiment the use comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter wherein the pharmaceutical composition is as described in Table 1a-1 and wherein the cabotegravir provides a Ctau of at least 1.05 μg/mL.

In an embodiment the use comprises first administering a loading dose of the pharmaceutical composition to the human; 2 to 6 weeks after administering the loading dose, administering a maintenance dose of the pharmaceutical composition to the human; and then administering the maintenance dose every 15 to 24 weeks thereafter wherein the pharmaceutical composition is as described in Table 1a-2 and wherein the cabotegravir provide a Ctau of at least 1.05 g/mL.

In an embodiment the loading dose of the pharmaceutical composition comprises 1600 to 4000 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises 1000 to 1800 mg of cabotegravir.

In an embodiment the loading dose of the pharmaceutical composition comprises 3000 to 4000 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises 1400 to 1800 mg of cabotegravir.

In an embodiment the loading dose of the pharmaceutical composition comprises 3000 to 4000 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises 1400 to 1800 mg of cabotegravir wherein the pharmaceutical composition comprises cabotegravir, mannitol, PS80, and sodium CMC; wherein cabotegravir is present in the form of particles having a mass median diameter (X50) of between (and including) 2.5 μm and 10 μm and the pharmaceutical composition has a cabotegravir concentration of about 533 mg/mL.

In an embodiment the loading dose of the pharmaceutical composition comprises 3000 to 4000 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises 1400 to 1800 mg of cabotegravir wherein the pharmaceutical composition comprises cabotegravir, mannitol, PS80, and sodium CMC; wherein cabotegravir is present in the form of particles having a mass median diameter (X50) of between (and including) 2.5 μm and 10 μm and the pharmaceutical composition has a cabotegravir concentration of about 400 mg/mL.

In an embodiment the loading dose of the pharmaceutical composition comprises about 3200 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises about 1600 mg of cabotegravir wherein the pharmaceutical composition comprises cabotegravir, mannitol, PS80, and sodium CMC; wherein cabotegravir is present in the form of particles having a mass median diameter (X50) of between (and including) 2.5 μm and 10 μm and the pharmaceutical composition has a cabotegravir concentration of about 533 mg/mL.

In an embodiment the loading dose of the pharmaceutical composition comprises about 3200 mg of cabotegravir; the first maintenance dose is administered 1 months i.e. 3 to 5 weeks after administering the loading dose, then the maintenance dose is administered every 4 months i.e. 16 to 18 weeks thereafter and the maintenance dose of the pharmaceutical composition comprises about 1600 mg of cabotegravir wherein the pharmaceutical composition comprises cabotegravir, mannitol, PS80, and sodium CMC; wherein cabotegravir is present in the form of particles having a mass median diameter (X50) of between (and including) 2.5 μm and 10 μm and the pharmaceutical composition has a cabotegravir concentration of about 400 mg/mL.

In a fifth aspect, there is provided the use of a pharmaceutical composition in the manufacture of a medicament for treating an HIV infection. The cabotegravir, doses of cabotegravir, method of administration, loading dose(s), maintenance dose, oral lead-in and oral bridging, pharmaceutical compositions, coadministration, and exemplary embodiments, are as described hereinabove for the first, second, third, and fourth aspects.

In a sixth aspect, there is provided the use of a pharmaceutical composition in the manufacture of a medicament for preventing an HIV infection. The cabotegravir, doses of cabotegravir, method of administration, loading dose(s), maintenance dose, oral lead-in and oral bridging, pharmaceutical compositions, coadministration, and exemplary embodiments, are as described hereinabove for the first, second, third, and fourth aspects.

The following numbered embodiments are exemplary and non-limiting:
Embodiment 1: A method of preventing HIV in a human, comprising administering to said human a pharmaceutical composition comprising cabotegravir once every 15 to 24 weeks.
Embodiment 2: A method of treating HIV in a human in need thereof, comprising administering to said human a pharmaceutical composition comprising cabotegravir once every 15 to 24 weeks.
Embodiment 3: The method according to Embodiment 1 or Embodiment 2, wherein the cabotegravir provides Ctau of at least 1 μg/mL.
Embodiment 4: The method according to Embodiment 3, wherein the human was assigned female at birth and the cabotegravir provides a Ctau of at least about 1.39 μg/mL; or wherein the human was assigned male at birth and the cabotegravir provides a Ctau of at least about 1.05 μg/mL.
Embodiment 5: The method according to Embodiment 1 or Embodiment 2, wherein the cabotegravir provides a Ctau higher than the Ctau achieved in the dosing regimen of APRETUDE®.
Embodiment 6: The method according to any preceding Embodiment, wherein the method comprises:
   a. first administering a loading dose of the pharmaceutical composition to the human;
   b. 2 to 6 weeks after administering the loading dose, administering a maintenance dose of the pharmaceutical composition to the human; and then
   c. administering the maintenance dose every 15 to 24 weeks thereafter.
Embodiment 7: The method according to Embodiment 6, wherein the loading dose of the pharmaceutical composition comprises at least 1600 mg of cabotegravir.
Embodiment 8: The method according to Embodiment 7, wherein the loading dose of the pharmaceutical composition comprises about 1600 mg to about 4000 mg of cabotegravir.
Embodiment 9: The method according to Embodiment 8, wherein the loading dose of the pharmaceutical composition comprises about 3200 mg of cabotegravir.
Embodiment 10: The method according to Embodiment 9, wherein the loading dose of the pharmaceutical composition is administered in two separate injections, each comprising about 1600 mg of cabotegravir.
Embodiment 11: The method according to any one of Embodiments 6 to 10, wherein the maintenance dose of the pharmaceutical composition comprises at least 1050 mg of cabotegravir.
Embodiment 12: The method according to Embodiment 11, wherein the maintenance dose of the pharmaceutical composition comprises about 1300 to about 1700 mg of cabotegravir.
Embodiment 13: The method according to Embodiment 12, wherein the maintenance dose of the pharmaceutical composition comprises about 1550 to about 1650 mg of cabotegravir.
Embodiment 14: The method according to Embodiment 12, wherein the maintenance dose of the pharmaceutical composition comprises about 1600 mg of cabotegravir.
Embodiment 15: The method according to any one of Embodiments 6 to 14, wherein the maintenance dose is administered once every 15 to 19 weeks.
Embodiment 16: The method according to Embodiment 15, wherein the maintenance dose is administered once every 4 months i.e. every 16 to 18 weeks.
Embodiment 17: The method according to any preceding Embodiment, wherein the pharmaceutical composition further comprises polysorbate 80.
Embodiment 18: The method according to any preceding Embodiment, wherein the pharmaceutical composition further comprises sodium carboxymethylcellulose.
Embodiment 19: The method according to any preceding Embodiment, wherein the pharmaceutical composition further comprises mannitol.
Embodiment 20: The method according to any preceding Embodiment, wherein the cabotegravir in the pharmaceutical composition is present in the form of particles with an X90 value greater than or equal to 5 μm and less than or equal to 25 μm.
Embodiment 21: The method according to any preceding Embodiment, wherein the cabotegravir in the pharmaceutical composition is present in the form of particles with an X50 value of between (and including) to 2.5 μm and 10 μm.
Embodiment 22: The method according to Embodiment 20 or 21, wherein the cabotegravir particles have an X50 value greater than or equal to 3 μm and less than or equal to 8.5 μm, and wherein the cabotegravir particles have an X90 value greater than or equal to 6 μm and less than or equal to 20 μm.
Embodiment 23: The method according to Embodiment 21, wherein the cabotegravir particles have an X50 value greater than or equal to 3.5 μm and less than or equal to 8.0 μm.
Embodiment 24: The method according to Embodiment 23, wherein the cabotegravir particles have an X50 value greater than or equal to 3.5 μm and less than or equal to 8.0 μm, and wherein the cabotegravir particles have an X90 value greater than or equal to 7.0 μm and less than or equal to 18.0 μm.
Embodiment 25: The method according to any preceding Embodiment, wherein the pharmaceutical composition comprises polysorbate 80, sodium carboxymethylcellulose, and mannitol, and wherein a weight ratio of cabotegravir: PS80: sodium CMC:mannitol is about 100:1:1.25:8.75.
Embodiment 26: The method according to any preceding Embodiment, wherein the pharmaceutical composition comprises polysorbate 80, sodium carboxymethylcellulose, and mannitol, and wherein a weight ratio of cabotegravir: PS80: sodium CMC:mannitol is about 400:3:3.7:25.9.
Embodiment 27: The method according to any preceding Embodiment, wherein a concentration of cabotegravir in the pharmaceutical composition is in a range of from about 300 mg/ml to about 650 mg/mL.
Embodiment 28: The method according to Embodiment 27, wherein the concentration of cabotegravir is about 533 mg/mL.
Embodiment 29: The method according to any preceding Embodiment, wherein the pharmaceutical composition has been reconstituted from a lyophilized powder with a suitable liquid.
Embodiment 30: The method according to any preceding Embodiment, wherein the pharmaceutical composition is administered intramuscularly or subcutaneously via injection.
Embodiment 31: The method according to Embodiment 30, wherein the pharmaceutical composition is administered intramuscularly.
Embodiment 32: The method according to Embodiment 31, wherein the pharmaceutical composition is administered in the gluteus medius.
Embodiment 33: The method according to any preceding Embodiment, wherein the human is screened for HIV before administration of the pharmaceutical composition.
Embodiment 34: A pharmaceutical composition for use in the treatment of HIV, wherein the pharmaceutical composition comprises cabotegravir and the treatment comprises administering the pharmaceutical composition to a human once every 15 to 24 weeks.
Embodiment 35: A pharmaceutical composition for use in the prevention of HIV, wherein the pharmaceutical composition comprises cabotegravir and the prevention comprises said prevention comprising administering the pharmaceutical composition to a human once every 15 to 24 weeks.
Embodiment 36: The pharmaceutical composition for use according to Embodiment 34 or Embodiment 35, wherein the cabotegravir provides Ctau of at least 1 μg/mL.
Embodiment 37: The pharmaceutical composition for use according to Embodiment 36, wherein the human was assigned female at birth and the cabotegravir provides a Ctau of at least about 1.39 μg/mL; or wherein the human was assigned male at birth and the cabotegravir provides a Ctau of at least about 1.05 μg/mL.
Embodiment 38: The pharmaceutical composition for use according to Embodiment 34 or Embodiment 35, wherein the cabotegravir provides a Ctau higher than the Ctau achieved in the dosing regimen of APRETUDE®.
Embodiment 39: The pharmaceutical composition for use according to Embodiment 34 or 35, wherein the use comprises:
   a. first administering a loading dose of the pharmaceutical composition to the human;
   b. 2 to 6 weeks after administering the loading dose, administering a maintenance dose of the pharmaceutical composition to the human; and then
   c. administering the maintenance dose every 15 to 24 weeks thereafter.
Embodiment 40: The pharmaceutical composition for use according to Embodiment 39 wherein the loading dose of the pharmaceutical composition comprises at least 1600 mg of cabotegravir.
Embodiment 41: The pharmaceutical composition for use according to Embodiment 40, wherein the loading dose of the pharmaceutical composition comprises about 1600 mg to about 4000 mg of cabotegravir.
Embodiment 42: The pharmaceutical composition for use according to Embodiment 41, wherein the loading dose of the pharmaceutical composition comprises about 3200 mg of cabotegravir.
Embodiment 43: The pharmaceutical composition for use according to Embodiment 42, wherein the loading dose of the pharmaceutical composition is administered in two separate injections, each comprising about 1600 mg of cabotegravir.
Embodiment 44: The pharmaceutical composition for use according to any one of Embodiments 34 to 43, wherein the maintenance dose of the pharmaceutical composition comprises at least 1050 mg of cabotegravir.
Embodiment 45: The pharmaceutical composition for use according to Embodiment 44, wherein the maintenance dose of the pharmaceutical composition comprises about 1300 to about 1800 mg of cabotegravir.
Embodiment 46: The pharmaceutical composition for use according to Embodiment 45, wherein the maintenance dose of the pharmaceutical composition comprises about 1550 to about 1650 mg of cabotegravir.
Embodiment 47: The pharmaceutical composition for use according to Embodiment 46, wherein the maintenance dose of the pharmaceutical composition comprises about 1600 mg of cabotegravir.
Embodiment 48: The pharmaceutical composition for use according to any one of Embodiments 34 to 47, wherein the maintenance dose is administered once every 15 to 20 weeks.
Embodiment 49: The pharmaceutical composition for use according to Embodiment 48, wherein the maintenance dose is administered once every 4 months i.e. every 16 to 18 weeks.
Embodiment 50: The pharmaceutical composition for use according to any one of Embodiments 34 to 49, wherein the pharmaceutical composition further comprises polysorbate 80.
Embodiment 51: The pharmaceutical composition for use according to any one of Embodiments 34 to 50, wherein the pharmaceutical composition further comprises sodium carboxymethylcellulose.
Embodiment 52: The pharmaceutical composition for use according to any one of Embodiments 34 to 51, wherein the pharmaceutical composition further comprises mannitol.
Embodiment 53: The pharmaceutical composition for use according to any one of Embodiments 34 to 52, wherein the cabotegravir in the pharmaceutical composition is present in the form of particles with an X90 value greater than or equal to 5 μm and less than or equal to 25 μm.
Embodiment 54: The pharmaceutical composition for use according to any one of Embodiments 34 to 53, wherein the cabotegravir in the pharmaceutical composition is present in the form of particles with an X50 value greater than or equal to 3 μm and less than or equal to 8.5 μm.
Embodiment 55: The pharmaceutical composition for use according to Embodiment 53 or 54, wherein the cabotegravir particles have an X50 value greater than or equal to 3 μm and less than or equal to 8.5 μm, and wherein the cabotegravir particles have an X90 value greater than or equal to 6 μm and less than or equal to 20 μm.
Embodiment 56: The pharmaceutical composition for use according to Embodiment 55, wherein the cabotegravir particles have an X50 value greater than or equal to 3.5 μm and less than or equal to 8.0 μm.
Embodiment 57: The pharmaceutical composition for use according to Embodiment 56, wherein the cabotegravir particles have an X50 value greater than or equal to 3.5 μm and less than or equal to 8.0 μm, and wherein the cabotegravir particles have an X90 value greater than or equal to 7.0 μm and less than or equal to 18.0 μm.
Embodiment 58: The pharmaceutical composition for use according to any one of Embodiments 34 to 57, wherein the pharmaceutical composition comprises polysorbate 80, sodium carboxymethylcellulose, and mannitol, and wherein a weight ratio of cabotegravir: PS80: sodium CMC:mannitol is about 100:1:1.25:8.75.
Embodiment 59: The pharmaceutical composition for use according to any one of Embodiments 34 to 58, wherein the pharmaceutical composition comprises polysorbate 80, sodium carboxymethylcellulose, and mannitol, and wherein a weight ratio of cabotegravir: PS80: sodium CMC:mannitol is about 400:3:3.7:25.9.
Embodiment 60: The pharmaceutical composition for use according to any one of Embodiments 34 to 59, wherein a concentration of cabotegravir in the pharmaceutical composition is in a range of from about 300 mg/mL to about 650 mg/mL.
Embodiment 61: The pharmaceutical composition for use according to Embodiment 60, wherein the concentration of cabotegravir is about 533 mg/mL.
Embodiment 62: The pharmaceutical composition for use according to any one of Embodiments 34 to 61, wherein the pharmaceutical composition has been reconstituted from a lyophilized powder with a suitable liquid.
Embodiment 63: The pharmaceutical composition for use according to any one of Embodiments 34 to 62, wherein the pharmaceutical composition is administered intramuscularly or subcutaneously via injection.
Embodiment 64: The pharmaceutical composition for use according to Embodiments 63, wherein the pharmaceutical composition is administered intramuscularly.
Embodiment 65: The pharmaceutical composition for use according to Embodiments 64, wherein the pharmaceutical composition is administered in the gluteus medius.
Embodiment 66: The pharmaceutical composition for use according to any one of Embodiments 34 to 65, wherein the human is screened for HIV before administration of the pharmaceutical composition.
Embodiment 67: A method of preventing or treating HIV infection in a human, or cabotegravir for use in prevention or treatment of HIV infection in a human, wherein the method or use comprises:
   a. administering to the human a first loading dose, wherein the first loading dose comprises a pharmaceutical composition comprising cabotegravir in a concentration of from about 300 mg/mL to about 650 mg/mL (pharmaceutical composition A) or a pharmaceutical composition comprising cabotegravir in a concentration of from about 150 mg/ml to about 230 mg/ml (pharmaceutical composition B);
   b. administering to the human a second loading dose, wherein the second loading dose comprises the pharmaceutical composition A;
   c. administering to the human a third loading dose, wherein the third loading dose comprises the pharmaceutical composition A;
   d. administering to the human a maintenance dose of the pharmaceutical composition A; and
   e. administering the maintenance dose of the pharmaceutical composition A every 15-24 weeks thereafter.
Embodiment 68: The method or cabotegravir for use according to Embodiment 67, wherein step (b) occurs 2-6 weeks after step (a); step (c) occurs 6-11 weeks after step (b); step (d) occurs 15-19 weeks after step (c); and step (e) begins 15-19 weeks after step (d) and occurs every 15-24 weeks thereafter.
Embodiment 69: The method or cabotegravir for use according to Embodiment 67, wherein step (b) occurs 3-5 weeks after step (a); step (c) occurs 7-10 weeks after step (b); step (d) occurs 16-18 weeks after step (c); and step (e) begins 16-18 weeks after step (d) and occurs every 16-18 weeks thereafter.
Embodiment 70: The method or cabotegravir for use according to any one of Embodiments 67-69, wherein the first loading dose comprises the pharmaceutical composition A.
Embodiment 71: The method or cabotegravir for use according to any one of Embodiments 67-69, wherein the first loading dose comprises the pharmaceutical composition B.
Embodiment 72: A method of preventing or treating HIV infection in a human, or cabotegravir for use in prevention or treatment of HIV infection in a human, wherein the method or use comprises:
   a. administering to the human a first loading dose, wherein the first loading dose comprises a pharmaceutical composition comprising cabotegravir in a concentration of from about 300 mg/mL to about 650 mg/ml (pharmaceutical composition A) or a pharmaceutical composition comprising cabotegravir in a concentration of from about 150 mg/ml to about 230 mg/mL (pharmaceutical composition B);
   b. administering to the human a second loading dose, wherein the second loading dose comprises the pharmaceutical composition A;
   c. administering to the human a maintenance dose of the pharmaceutical composition A; and
   d. administering the maintenance dose of the pharmaceutical composition A every 15-24 weeks.
Embodiment 73: The method or cabotegravir for use according to Embodiment 72, wherein step (b) occurs 15-19 weeks after step (a); step (c) occurs 15-19 weeks after step (b); and step (d) begins 15-19 weeks after step (c) and occurs every 15-19 weeks thereafter.
Embodiment 74: The method or cabotegravir for use according to Embodiment 72, wherein step (b) occurs 16-18 weeks after step (a); step (c) occurs 16-18 weeks after step (b); and step (d) begins 16-18 weeks after step (c) and occurs every 16-18 weeks thereafter.
Embodiment 75: A method of preventing or treating HIV infection in a human, or cabotegravir for use in prevention or treatment of HIV infection in a human, wherein the method or use comprises:
   a. administering to the human a first loading dose, wherein the first loading dose comprises a pharmaceutical composition comprising cabotegravir in a concentration of from about 300 mg/mL to about 650 mg/ml (pharmaceutical composition A);
   b. administering to the human a second loading dose, wherein the second loading dose comprises the pharmaceutical composition A;
   c. administering to the human a maintenance dose of the pharmaceutical composition A; and
   d. administering the maintenance dose of the pharmaceutical composition A every 15-24 weeks thereafter.
Embodiment 76: The method or cabotegravir for use according to Embodiment 75, wherein step (b) occurs 6-11 weeks after step (a); step (c) occurs 15-19 weeks after step (b); and step (d) begins 15-19 weeks after step (c) and occurs every 15-19 weeks thereafter.
Embodiment 77: The method or cabotegravir for use according to Embodiment 75, wherein step (b) occurs 7-10 weeks after step (a); step (c) occurs 16-18 weeks after step (b); and step (d) begins 16-18 weeks after step (c) and occurs every 16-18 weeks thereafter.
Embodiment 78: A method of preventing or treating HIV infection in a human, or cabotegravir for use in prevention or treatment of HIV infection in a human, wherein the method or use comprises:
   a. administering to the human a first loading dose, wherein the first loading dose comprises a pharmaceutical composition comprising cabotegravir in a concentration of from about 300 mg/mL to about 630 mg/ml (pharmaceutical composition A) and a pharmaceutical composition comprising cabotegravir in a concentration of from about 150 mg/ml to about 230 mg/ml (pharmaceutical composition B), wherein the pharmaceutical composition A and the pharmaceutical composition B are injected separately;
   b. administering to the human a second loading dose, wherein the second loading dose comprises the pharmaceutical composition A;
   c. administering to the human a third loading dose, wherein the third loading dose comprises the pharmaceutical composition A;
   d. administering the third loading dose, administering to the human a maintenance dose of the pharmaceutical composition A; and
   e. administering the maintenance dose of the pharmaceutical composition A every 15-24 weeks thereafter.
Embodiment 79: The method or cabotegravir for use according to Embodiment 78, wherein step (b) occurs 6-11 weeks after step (a); step (c) occurs 6-11 weeks after step (b); step (d) occurs 15-19 weeks after step (c); and step (e) begins 15-19 weeks after step (d) and occurs every 15-19 weeks thereafter.
Embodiment 80: The method or cabotegravir for use according to Embodiment 78, wherein step (b) occurs 7-10 weeks after step (a); step (c) occurs 7-10 weeks after step (b); step (d) occurs 16-18 weeks after step (c); and step (e) begins 16-18 weeks after step (d) and occurs every 16-18 weeks thereafter.
Embodiment 81: A method of preventing or treating HIV infection in a human, or cabotegravir for use in prevention or treatment of HIV infection in a human, wherein the method or use comprises:
   a. administering to the human a first loading dose, wherein the first loading dose comprises a pharmaceutical composition comprising cabotegravir in a concentration of from about 150 mg/mL to about 230 mg/ml (pharmaceutical composition B);
   b. administering to the human a second loading dose, wherein the second loading dose comprises the pharmaceutical composition B;
   c. administering to the human a third loading dose, wherein the third loading dose comprises a pharmaceutical composition comprising cabotegravir in a concentration of from about 300 mg/mL to about 650 mg/mL (pharmaceutical composition A);
   d. administering to the human a fourth loading dose, wherein the fourth loading dose comprises the pharmaceutical composition A;
   e. administering to the human a maintenance dose of the pharmaceutical composition A; and
   f. administering the maintenance dose of the pharmaceutical composition A every 15-24 weeks thereafter.
Embodiment 82: The method or cabotegravir for use according to Embodiment 81, wherein step (b) occurs 2-6 weeks after step (a); step (c) occurs 6-11 weeks after step (b); step (d) occurs 6-11 weeks after step (c); step (e) occurs 15-19 weeks after step (d); and step (f) begins 15-19 weeks after step (e) and occurs every 15-19 weeks thereafter.
Embodiment 83: The method or cabotegravir for use according to Embodiment 81, wherein step (b) occurs 3-5 weeks after step (a); step (c) occurs 7-10 weeks after step (b); step (d) occurs 7-10 weeks after step (c); step (e) occurs 16-18 weeks after step (d); and step (f) begins 16-18 weeks after step (e) and occurs every 16-18 weeks thereafter.
Embodiment 84: A method of preventing or treating HIV infection in a human, or cabotegravir for use in prevention or treatment of HIV infection in a human, wherein the method or use comprises:
   a. administering to the human a first loading dose, wherein the first loading dose comprises a pharmaceutical composition comprising cabotegravir in a concentration of from about 300 mg/mL to about 650 mg/ml (pharmaceutical composition A);
   b. administering to the human a maintenance dose of the pharmaceutical composition A; and
   c. administering the maintenance dose of the pharmaceutical composition A every 15-24 weeks.
Embodiment 85: The method or cabotegravir for use according to Embodiment 84, wherein step (b) occurs 6-11 weeks after step (a); and step (c) begins 15-19 weeks after step (b) and occurs every 15-19 weeks thereafter.
Embodiment 86: The method or cabotegravir for use according to Embodiment 84, wherein step (b) occurs 7-10 weeks after step (a); and step (c) begins 16-18 weeks after step (b) and occurs every 16-18 weeks thereafter.
Embodiment 87: The method or cabotegravir for use of any one of Embodiments 67-86, wherein the first loading dose comprises at least about 600 mg of cabotegravir.
Embodiment 88: The method or cabotegravir for use of Embodiment 70, wherein the first loading dose comprises about 1600 mg of cabotegravir, the second loading dose comprises about 1600 mg of cabotegravir, the third loading dose comprises about 1600 mg of cabotegravir, and each maintenance doses comprises about 1600 mg of cabotegravir.
Embodiment 89: The method or cabotegravir for use of Embodiment 70 or 88, wherein the first loading dose comprises 1600 mg of cabotegravir, the second loading dose comprises 1600 mg of cabotegravir, the third loading dose comprises 1600 mg of cabotegravir, and each maintenance dose comprises 1600 mg of cabotegravir.
Embodiment 90: The method or cabotegravir for use of Embodiment 71, wherein the first loading dose comprises about 600 mg of cabotegravir, the second loading dose comprises about 1600 mg of cabotegravir, the third loading dose comprises about 1600 mg of cabotegravir, and each maintenance dose comprises about 1600 mg of cabotegravir.
Embodiment 91: The method or cabotegravir for use of Embodiment 71 or 90, wherein the first loading dose comprises 600 mg of cabotegravir, the second loading dose comprises 1600 mg of cabotegravir, the third loading dose comprises 1600 mg of cabotegravir, and each maintenance dose comprises 1600 mg of cabotegravir.
Embodiment 92: The method or cabotegravir for use of any one of Embodiments 72-74 wherein the first loading dose comprises about 3200 mg of cabotegravir, the second loading dose comprises about 1600 mg of cabotegravir, and each maintenance dose comprises about 1600 mg of cabotegravir.
Embodiment 93: The method or cabotegravir for use of any one of Embodiments 72-74 and 92, wherein the first loading dose comprises 3200 mg of cabotegravir, the second loading dose comprises 1600 mg of cabotegravir, and each maintenance dose comprises 1600 mg of cabotegravir.
Embodiment 94: The method or cabotegravir for use of any one of Embodiments 75-77, wherein the first loading dose comprises about 2400 mg of cabotegravir, the second loading dose comprises about 1600 mg of cabotegravir, and each maintenance dose comprises about 1600 mg of cabotegravir.
Embodiment 95: The method or cabotegravir for use of any one of Embodiments 75-77 and 94, wherein the first loading dose comprises 2400 mg of cabotegravir, the second loading dose comprises 1600 mg of cabotegravir, and each maintenance dose comprises 1600 mg of cabotegravir.
Embodiment 96: The method or cabotegravir for use of any one of Embodiments 78-80, wherein the first loading dose comprises the pharmaceutical composition A containing about 1600 mg of cabotegravir and the pharmaceutical composition B containing about 600 mg of cabotegravir, the second loading dose comprises about 1600 mg of cabotegravir, the third loading dose comprises about 1600 mg of cabotegravir, and each maintenance dose comprises about 1600 mg of cabotegravir.
Embodiment 97: The method or cabotegravir for use of any one of Embodiments 78-80 and 96, wherein the first loading dose comprises the pharmaceutical composition A containing 1600 mg of cabotegravir and the pharmaceutical composition B containing 600 mg of cabotegravir, the second loading dose comprises 1600 mg of cabotegravir, the third loading dose comprises 1600 mg of cabotegravir, and each maintenance dose comprises 1600 mg of cabotegravir.
Embodiment 98: The method or cabotegravir for use of any one of Embodiments 81-83, wherein the first loading dose comprises about 600 mg of cabotegravir, the second loading dose comprises about 600 mg of cabotegravir, the third loading dose comprises about 1600 mg of cabotegravir, the fourth loading dose comprises about 1600 mg of cabotegravir, and each maintenance dose comprises about 1600 mg of cabotegravir.
Embodiment 99: The method or cabotegravir for use of any one of Embodiments 81-83 and 98, wherein the first loading dose comprises 600 mg of cabotegravir, the second loading dose comprises 600 mg of cabotegravir, the third loading dose comprises 1600 mg of cabotegravir, the fourth loading dose comprises 1600 mg of cabotegravir, and each maintenance dose comprises 1600 mg of cabotegravir.
Embodiment 100: The method or cabotegravir for use of any one of Embodiments 84-86, wherein the first loading dose comprises about 1600 mg of cabotegravir, and each maintenance dose comprises about 1600 mg of cabotegravir.
Embodiment 101: The method or cabotegravir for use of any one of Embodiments 84-86 and 100, wherein the first loading dose comprises 1600 mg of cabotegravir, and each maintenance dose comprises 1600 mg of cabotegravir.
Embodiment 102: The method or cabotegravir for use according to any one of Embodiments 67-69, 71, 78-83, 90, 91, and 98-101, wherein the pharmaceutical composition B comprises cabotegravir in a concentration of about 200 mg/mL.
Embodiment 103: The method or cabotegravir for use according to any one of Embodiments 67-69, 71, 78-83, 90, 91, and 98-102, wherein the pharmaceutical composition B comprises cabotegravir in a concentration of 200 mg/mL.
Embodiment 104: The method or cabotegravir for use according to any one of Embodiments 67-103, wherein the pharmaceutical composition A comprises cabotegravir is in a concentration of from about 400 mg/mL to about 550 mg/mL.
Embodiment 105: The method or cabotegravir for use according to any one of Embodiments 67-104, wherein the pharmaceutical composition A comprises cabotegravir is in a concentration of about 400 mg/mL.
Embodiment 106: The method or cabotegravir for use according to any one of Embodiments 67-105, wherein the pharmaceutical composition A comprises cabotegravir is in a concentration of 400 mg/mL.
Embodiment 107: The method or cabotegravir for use according to any one of Embodiments 67-104, wherein the pharmaceutical composition A comprises cabotegravir is in a concentration of about 533 mg/mL.
Embodiment 108: The method or cabotegravir for use according to any one of Embodiments 67-104 and 107, wherein the pharmaceutical composition A comprises cabotegravir is in a concentration of 533 mg/mL.
Embodiment 109: The method or cabotegravir for use according to any one of Embodiments 67-108, wherein the pharmaceutical composition A further comprises polysorbate 80.
Embodiment 110: The method or cabotegravir for use according to any one of Embodiments 67-109, wherein the pharmaceutical composition A further comprises sodium carboxymethylcellulose.
Embodiment 111: The method or cabotegravir for use according to any one of Embodiments 67-110, wherein the pharmaceutical composition A further comprises mannitol.
Embodiment 112: The method or cabotegravir for use according to any one of Embodiments 67-111, wherein the cabotegravir in the pharmaceutical composition A is present in the form of particles with an X90 value greater than or equal to 5 μm and less than or equal to 25 μm.
Embodiment 113: The method or cabotegravir for use according to any one of Embodiments 67-112, wherein the cabotegravir in the pharmaceutical composition A is present in the form of particles with an X50 value of between (and including) to 2.5 μm and 10 μm.
Embodiment 114: The method or cabotegravir for use according to Embodiment 112 or 113, wherein the cabotegravir particles in the pharmaceutical composition A have an X50 value greater than or equal to 3 μm and less than or equal to 8.5 μm, and wherein the cabotegravir particles in the pharmaceutical composition A have an X90 value greater than or equal to 6 μm and less than or equal to 20 μm.
Embodiment 115: The method or cabotegravir for use according to any one of Embodiments 112-114, wherein the cabotegravir particles have an X50 value greater than or equal to 3.5 μm and less than or equal to 8.0 μm.
Embodiment 116: The method or cabotegravir for use according to Embodiment 115, wherein the cabotegravir particles have an X50 value greater than or equal to 3.5 μm and less than or equal to 8.0 μm, and wherein the cabotegravir particles have an X90 value greater than or equal to 7.0 μm and less than or equal to 18.0 μm.
Embodiment 117: The method or cabotegravir for use according to any one of Embodiments 67-116, wherein the pharmaceutical composition A comprises polysorbate 80, sodium carboxymethylcellulose, and mannitol, a weight ratio of and wherein cabotegravir: PS80: sodium CMC:mannitol is about 100:1:1.25:8.75.
Embodiment 118: The method or cabotegravir for use according to any one of Embodiments 67-116, wherein the pharmaceutical composition A comprises polysorbate 80, sodium carboxymethylcellulose, and mannitol, and wherein a weight ratio of cabotegravir: PS80: sodium CMC:mannitol is about 400:3:3.7:25.9.
Embodiment 119: The method or cabotegravir for use according to any one of Embodiments 67-118, wherein the pharmaceutical composition A has been reconstituted from a lyophilized powder with a suitable liquid.
Embodiment 120: The method or cabotegravir for use according to any one of Embodiments 67-119, wherein the pharmaceutical composition A is administered intramuscularly or subcutaneously via injection, and, if present, the pharmaceutical composition B is administered intramuscularly or subcutaneously via injection.
Embodiment 121: The method or cabotegravir for use according to Embodiment 120, wherein the pharmaceutical composition A is administered intramuscularly, and, if present, the pharmaceutical composition B is administered intramuscularly.
Embodiment 122: The method or cabotegravir for use according to Embodiment 121, wherein the pharmaceutical composition A is administered in the gluteus medius, and if present, the pharmaceutical composition B is administered in the gluteus medius.
Embodiment 123: The method or cabotegravir for use according to Embodiment 120, wherein the pharmaceutical composition A is administered subcutaneously, and, if present, the pharmaceutical composition B is administered subcutaneously.
Embodiment 124: The method or cabotegravir for use according to any one of Embodiments 67-123, wherein the human is screened for HIV before administration of the first loading dose.
Embodiment 125: The method or cabotegravir for use according to any one of Embodiments 67-124, wherein cabotegravir provides Ctau of at least 1 μg/mL.
Embodiment 126: The method or cabotegravir for use according to any one of Embodiments 67-125, wherein the human was assigned female at birth and cabotegravir provides a Ctau of at least about 1.39 μg/mL; or wherein the human was assigned male at birth and cabotegravir provides a Ctau of at least about 1.05 μg/mL.
Embodiment 127: The method according to any one of Embodiments 67-126 wherein cabotegravir provides a Ctau higher than the Ctau achieved in the dosing regimen of APRETUDE®.
Embodiment 128: The method according to any one of Embodiments 67-127, wherein the method is for preventing HIV infection in a human.
Embodiment 129: The method according to any one of Embodiments 67-127, wherein the method is for treating HIV infection in a human.
Embodiment 130: The cabotegravir for use according to any one of Embodiments 67-127, wherein the cabotegravir for use is for prevention of HIV infection in a human.
Embodiment 131: The cabotegravir for use according to any one of Embodiments 67-127, wherein the cabotegravir for use is for treatment of HIV infection in a human.
Embodiment 132: The method according to claim 30, wherein when two separate injections are administered in one loading dose or in one maintenance dose, one of said separate injections is administered intramuscularly and the other of said injections is administered subcutaneously.
Embodiment 133: The method according to claim 30, wherein when two separate injections are administered in one loading dose or in one maintenance dose, said separate injections are both administered intramuscularly.
Embodiment 134: The method according to claim 30, wherein when two separate injections are administered in one loading dose or in one maintenance dose, said separate injections are both administered subcutaneously.
Embodiment 135: The method according to any one of Embodiments 32 and 132-134, wherein when two separate injections are administered, said separate injections are administered to different sites at least 2 cm apart.
Embodiment 136: The method according to Embodiment 31, wherein the pharmaceutical composition is administered in the gluteus medius, the gluteus maximus, or both the gluteus medius and the gluteus maximus.
Embodiment 137: The method according to Embodiment 136, wherein the pharmaceutical composition is administered in the gluteus maximus.
Embodiment 138: The method according to Embodiment 137, wherein when two separate injections are administered, one of said separate injections is administered in the left gluteus maximus and the other of said separate injections is administered in the right gluteus maximus.
Embodiment 139: The method according to Embodiment 32, wherein when two separate injections are administered, one of said separate injections is administered in the left gluteus medius and the other of said separate injections is administered in the right gluteus medius.
Embodiment 140: Use of a pharmaceutical composition in the manufacture of a medicament for treating an HIV infection, wherein the pharmaceutical composition comprises cabotegravir.
Embodiment 141: Use of a pharmaceutical composition in the manufacture of a medicament for preventing an HIV infection, wherein the pharmaceutical composition comprises cabotegravir.

The following non-limiting examples illustrate the present invention.

### Examples

In the following description of the Examples, specific embodiments are described. These embodiments are described in sufficient detail to enable those skilled in the art to practice certain embodiments of the present disclosure. Other embodiments may be utilized and logical and other changes may be made without departing from the scope of the disclosure. The following description is, therefore, not intended to limit the scope of the present disclosure.

### Comparative Example (Example 8 of WO 2021/116872A1)

A long-acting suspension comprising 200 mg/mL cabotegravir, 20 mg/mL PS20 and 20 mg/mL PEG3350 was investigated to test stability when the cabotegravir concentration was increased to 400 mg/mL.

The suspension was prepared in a 250 mL batch using a Netzsch miniCer and 0.3 mm YTZ grinding beads. A range of excipient concentrations were explored.

The formulation vehicle was prepared by dissolving Polysorbate 20 (Croda), Polyethylene Glycol 3350 (Clariant), and mannitol (Roquette Freres) in water for injection (WFI) and filtering the solution through a 0.2 μm filter. The formulation vehicle was then added to cabotegravir (free acid) to prepare a 400 mg/mL coarse suspension. The coarse suspension, while stirred, was circulated through a wet bead mill set at 29.7 Hz (Netzsch MiniCer) containing 0.30 mm YTZ grinding beads (Nikkato Corp) at 73-145 mL/min until the desired median particle diameter of less than 0.25 μm was reached as measured by laser diffraction. The wet bead mill was cooled to maintain a temperature between 1 and 25° C. The suspension was then filled into Type I glass vials, flushed with nitrogen, stoppered (FM457 stopper) and sealed. The filled suspension was terminally sterilized by gamma irradiation at a minimum dose of 25kGy.

30 mg/mL PS20, 20 mg/mL PEG3350 and 19 mg/mL mannitol with 400 mg/ml cabotegravir was prepared. The gamma irradiated suspension in vials was set down in the upright position and stored at 40° C./75% RH. After 1 month and 3 months of storage, suspensions were tested for suitability. Table 3 shows particle size (μm) after formulation and after 1 month of storage at 40° C./75% RH for the 400 mg/mL cabotegravir formulation with 30 mg/mL PS20, 30 mg/mL PEG3350, 19 mg/mL mannitol. At 1 month, the particle size had increased compared to the initial time point (Table 3). At 3 months, the suspension had become an unrecoverable gel which could not be removed from the vial with a syringe.

TABLE 3

| Time | X10 | X50 | X90 |
|---|---|---|---|
| Initial | 0.07 | 0.17 | 0.61 |
| 1 month | 0.14 | 0.41 | 3.2 |

Further suspensions comprising cabotegravir, PS20 and PEG3350 were prepared with the concentrations shown in Table 4. Experiments 1, 2, and 5 were prepared using the method described above for 20 mg/mL PS20 and PEG3350. In experiment 3, the suspension was first milled with PS20 (no PEG3350), and then PEG3350 was added, here 400 mg/mL cabotegravir was milled with 30 mg/mL PS20. After milling, a concentrated solution (400 mg/ml) of PEG3350 was added to dilute the 400 mg/mL suspension to 360 mg/mL cabotegravir, 27 mg/mL PS20, 27 mg/mL mannitol. Compositions were all prepared on a 250 mL batch scale using the Netzsch miniCer with 0.3 mm YTZ grinding beads.

TABLE 4

| Experiment Number | PS20 (mg/mL) | PEG3350 (mg/mL) |
|---|---|---|
| 1 | 40.0 | 40.0 |
| 2 | 30.0 | 40.0 |
| 3 | 27* | 40.0 |
| 4 | 20 | 20.0 |
| 5 | 20 | 0 |

*400 mg/mL was diluted down by 10%, resulting in 360 mg/mL cabotegravir and 27 mg/mL PS20

In experiments 1, 2, 4 and 5 during wet bead milling on the miniCer, the suspension thickened into a paste and was not recoverable. In experiment 3, within 5 minutes of adding the 400 mg/mL PEG3350 solution, the suspension thickened and could no longer be stirred. The combination of PS20 and PEG3350 with 400 mg/mL cabotegravir did not produce a physically stable suspension.

### Experimental Examples

In the following description of the Examples, specific embodiments are described. These embodiments are described in sufficient detail to enable those skilled in the art to practice certain embodiments of the present disclosure. Other embodiments may be utilized and logical and other changes may be made without departing from the scope of the disclosure. The following description is, therefore, not intended to limit the scope of the present disclosure.

Cabotegravir particle sizes in microsuspensions in the following examples were measured according to the below protocol description.

### Protocol Description

Carried out the method for Laser Diffraction Measurement of Particle Size (USP <429>).
Reconstituted the lyophilized product with water for injection (WFI) (volume of WFI determined by desired concentration of reconstituted suspension).
Cleaned and filled Malvern Mastersizer 3000 laser diffraction with MV instrument with deionized water.
Dispersion stirring speed was 2000 rpm, particle refractive index was 1.67, particle absorption Index (imaginary part of refractive index) was 0.01, General Purpose analysis mode with normal sensitivity, dispersant (DI water) refractive index was 1.33, measurement time and background time were both 10 seconds, and 2 measurements were acquired by the instrument per aliquot.
Performed an alignment and measured the background.
Agitated suspension (created in below examples) to ensure that all particles are suspended in the drug product vial.
Withdrew ~0.2 mL of content with a 1 ml syringe fitted with a needle (18 G).
Added two drops of the suspension into a microcentrifuge tube containing 0.5 mL of 6.6% w/v P338 in water solution.
Vortexed gently to create a homogenous dispersion of drug product in P338 dispersant.
Added appropriate amount of this dispersion to the instrument until obscuration of 5%-7% is achieved.
Allowed test suspension to circulate in the instrument for approximately 30 seconds prior to initiating the measurement.
Performed the measurement, and after each measurement flushed the instrument with deionized water.
Repeated this three times using a new aliquot of the sample each time and calculated the average values obtained from the volume distribution.

### Example 1

TABLE 5

| Ingredient | Concentration (mg/mL) |
|---|---|
| Cabotegravir, micronized | 400.0 |
| Sodium Carboxymethylcellulose | 3.7 |
| Polysorbate 80 | 3.0 |
| Mannitol | 25.9 |

Table 5 shows an exemplary pharmaceutical composition of the invention (pharmaceutical compositions are also described in these examples as “suspensions”), which was made using the following method.

A formulation vehicle was prepared by dissolving/diluting 24.0 g polysorbate 80 (“PS80”) (Croda) in about 300 g water. Separately, 30.0 g sodium carboxymethylcellulose (“NaCMC”) (Ashland, 7L2P) and 210.0 g mannitol (Roquette Freres) were dissolved in 4.8 kg water for injection (WFI). Once the NaCMC and mannitol were dissolved, the PS80 solution was added to the NaCMC-mannitol solution with stirring. The weighing and dilution vessels were rinsed into the compounding vessel with additional water and the compounded vehicle solution was brought to a final weight of 6.06 kg and filtered through a 0.2 μm filter. 1.6 kg Cabotegravir Micronized Free Acid (target X50=5-6 μm particle size) was added to 3.0 kg filtered vehicle and was mixed to form a homogeneous suspension. The compounded suspension was deaerated while stirring until it reached its target batch volume, and the suspension was then filled into vials. The product was lyophilized by freezing at −45° C. for at least 2 hours, annealing at −18° C. for at least 2 hours, refreezing at −45° C. for at least 2 hours (each transition at a ramp rate of +/−1° C./min), primary drying at −10° C. (ramp rate: 0.15° C./min) for at least 23 hours at approximately 150 mTorr, and secondary drying at 25° C. (ramp rate: 0.58° C./min) for at least 6 hours at approximately 150 mTorr. The lyophilized vials were backflushed with nitrogen to about 600 Torr, sealed, and sterilized by gamma irradiation at a minimum dose of 25kGy. The formulation is reconstituted with WFI and briefly shaken to resuspend prior to administration.

### Example 2

TABLE 6

| Ingredient | Concentration (mg/mL) |
|---|---|
| Cabotegravir, micronized | 400.0 |
| Sodium Carboxymethylcellulose | 5.0 |
| Polysorbate 80 | 4.0 |
| Mannitol | 35.0 |

Table 6 shows an exemplary pharmaceutical composition of the invention (pharmaceutical compositions are also described in these examples as “suspensions”), which was made using the following method.

A formulation vehicle was prepared by dissolving/diluting 24.0 g polysorbate 80 (“PS80”) (Croda) in about 300 g water. Separately, 30.0 g sodium carboxymethylcellulose (“NaCMC”) (Ashland, 7L2P) and 210.0 g mannitol (Roquette Freres) were dissolved in 3.4 kg water for injection (WFI). Once the NaCMC and mannitol were dissolved, the PS80 solution was added to the NaCMC-mannitol solution with stirring. The weighing and dilution vessels were rinsed into the compounding vessel with additional water and the compounded vehicle solution was brought to a final weight of 4.50 kg and filtered through a 0.2 μm filter. 1.6 kg Cabotegravir Micronized Free Acid (target X50=5-6 μm particle size) was added to 3.0 kg filtered vehicle and was mixed to form a homogeneous suspension. The compounded suspension was deaerated while stirring until it reached its target batch volume, and the suspension was then filled into vials. The product was lyophilized by freezing at −45° C. for at least 2 hours, annealing at −10° C. for at least 2 hours, refreezing at −45° C. for at least 2 hours (each transition at a ramp rate of +/−1° C./min), primary drying at −5° C. (ramp rate: 1° C./min) for at least 20 hours at approximately 150 mTorr, and secondary drying at 40° C. (ramp rate: 1° C./min) for at least 6 hours at approximately 150 mTorr. The lyophilized vials were backflushed with nitrogen to about 600 Torr, sealed, and sterilized by gamma irradiation at a minimum dose of 25kGy. The formulation is reconstituted with WFI and briefly shaken to resuspend prior to administration.

### Example 3

TABLE 7

| Ingredient | Concentration (mg/mL) |
|---|---|
| Cabotegravir, micronized | 400.0 |
| Sodium Carboxymethylcellulose | 5.0 |
| Polysorbate 80 | 4.0 |
| Mannitol | 35.0 |

Table 7 shows an exemplary pharmaceutical composition of the invention (pharmaceutical compositions are also described in these examples as “suspensions”), which was made using the following method.

A formulation vehicle was prepared by dissolving/diluting 24.0 g polysorbate 80 (“PS80”) (Croda) in about 300 g water. Separately, 30.0 g sodium carboxymethylcellulose (“NaCMC”) (Ashland, 7L2P) and 210.0 g mannitol (Roquette Freres) were dissolved in 3.4 kg water for injection (WFI). Once the NaCMC and mannitol were dissolved, the PS80 solution was added to the NaCMC-mannitol solution with stirring. The weighing and dilution vessels were rinsed into the compounding vessel with additional water and the compounded vehicle solution was brought to a final weight of 4.50 kg and filtered through a 0.2 μm filter. 1.6 kg Cabotegravir Micronized Free Acid (target X50=3-4 μm particle size) was added to 3.0 kg filtered vehicle and was mixed to form a homogeneous suspension. The compounded suspension was deaerated while stirring until it reached its target batch volume, and the suspension was then filled into vials. The product was lyophilized by freezing at −45° C. for at least 2 hours, annealing at −10° C. for at least 2 hours, refreezing at −45° C. for at least 2 hours (each transition at a ramp rate of +/−1° C./min), primary drying at −5° C. (ramp rate: 1° C./min) for at least 20 hours at approximately 150 mTorr, and secondary drying at 40° C. (ramp rate: 1° C./min) for at least 6 hours at approximately 150 mTorr. The lyophilized vials were backflushed with nitrogen to about 600 Torr, sealed, and sterilized by gamma irradiation at a minimum dose of 25kGy. The formulation was reconstituted with WFI and briefly shaken to resuspend prior to administration.

Table 8, below, applies to Examples 4-6.

TABLE 8

| Batch # | Formulation (when reconstituted) |
|---|---|
| 1 | Cabotegravir 400 mg/mL; 0.4 w/v % (4 mg/mL) PS80; 0.5 w/v % (5 |
| | mg/mL) NaCMC; 3.5 w/v % (35 mg/mL) Mannitol |
| 2 | Cabotegravir 400 mg/mL; 0.4 w/v % (4 mg/mL) PS20; 0.5 w/v % (5 |
| | mg/mL) NaCMC; 3.5 w/v % (35 mg/mL) Mannitol |
| 3 | Cabotegravir 400 mg/mL; 0.4 w/v % (4 mg/mL) P338; 0.5 w/v % (5 |
| | mg/mL) NaCMC; 3.5 w/v % (35 mg/mL) Mannitol |

### Example 4: Stability of a Composition Comprising Cabotegravir, Sodium Carboxymethylcellulose, Polysorbate 80 and Mannitol

A formulation vehicle was prepared by dissolving 2.10 g PS80 (Croda), 2.63 g NaCMC (Ashland), and 18.38 g mannitol (Roquette Freres) in 369.6 g WFI and filtering the solution through a 0.2 μm filter. The formulation vehicle was added to 210 g Cabotegravir (free acid) to prepare a 400 mg/mL coarse suspension. The suspension was covered and stirred for 2 hrs. The suspension was filled into Type I glass vials and lyophilized as described in Examples 1-3. The lyophilized suspension was reconstituted to a cabotegravir concentration of 400 mg/mL.

TABLE 9
Batch #1 (400 mg/mL cabotegravir; 0.4 w/v
% PS80; 0.5 w/v % NaCMC; 3.5 w/v % mannitol)

| | | Particle Size by | | |
|---|---|---|---|---|
| | | Laser Diffraction | | |
| Storage | Time | (microns) | | |
| Condition | (Months) | X10 | X50 | X90 |
| Initial | 0 | 3.1 | 7.6 | 15.6 |
| 30° C./75% | 1 | 3.2 | 7.7 | 15.4 |
| RH Upright | 3 | 3.2 | 7.5 | 14.3 |
| | 6 | 3.2 | 7.6 | 15.3 |
| | 9 | 3.2 | 7.5 | 14.8 |
| | 12 | 3.3 | 7.5 | 14.3 |
| 40° C./75% | 1 | 3.2 | 7.6 | 15.0 |
| RH Upright | 3 | 3.2 | 7.7 | 14.9 |
| | 6 | 3.1 | 7.3 | 13.9 |
| 50° C./AmbH | DY14 | 3.2 | 7.7 | 15.7 |
| Upright | 1 | 3.2 | 7.6 | 15.3 |
| | 3 | 3.3 | 7.7 | 14.7 |

Notes:
AmbH Ambient Humidity
RH Relative Humidity
— Denotes testing not scheduled at these timepoints
NGT = Not greater than
NLT = Not less than

### Example 5: Stability of a Composition Comprising Cabotegravir, sodium carboxymethylcellulose, polysorbate 20 and mannitol

A formulation vehicle was prepared by dissolving 2.10 g polysorbate 20 (Croda), 2.63 g sodium CMC (Ashland), and 18.38 g mannitol (Roquette Freres) in 550.9 g WFI and filtering the solution through a 0.2 μm filter. The formulation vehicle was added to 210 g cabotegravir (free acid) to prepare a 300 mg/mL coarse suspension. The suspension was covered and stirred for 2 hrs. The suspension was filled into Type I glass vials and lyophilized as described in Examples 1-3. The lyophilized suspension was reconstituted to a cabotegravir concentration of 400 mg/mL.

TABLE 10
Batch #2 (400 mg/mL cabotegravir; 0.4 w/v
% PS20; 0.5 w/v % NaCMC; 3.5 w/v % mannitol)

| | | Particle Size by | | |
|---|---|---|---|---|
| | | Laser Diffraction | | |
| Storage | Time | (microns) | | |
| Condition | (Months) | X10 | X50 | X90 |
| Initial | 0 | 3.0 | 7.4 | 14.9 |
| 30° C./75% | 1 | 3.0 | 7.4 | 14.8 |
| RH Upright | 3 | 3.0 | 7.4 | 15.0 |
| | 6 | 2.9 | 7.0 | 13.8 |
| | 9 | 3.0 | 7.3 | 14.0 |
| | 12 | 3.2 | 7.4 | 14.0 |
| 40° C./75% | 1 | 2.8 | 7.1 | 14.2 |
| RH Upright | 3 | 2.9 | 7.3 | 14.6 |
| | 6 | 2.9 | 7.1 | 14.4 |
| 50° C./AmbH | DY14 | 2.8 | 7.2 | 14.7 |
| Upright | 1 | 2.9 | 7.3 | 14.3 |
| | 3 | 3.0 | 7.4 | 14.4 |

Notes:
AmbH Ambient Humidity
RH Relative Humidity
— Denotes testing not scheduled at these timepoints
NGT = Not greater than
NLT = Not less than

### Example 6: Stability of a Composition Comprising Cabotegravir, sodium carboxymethylcellulose, poloxamer 338 and mannitol

A formulation vehicle was prepared by dissolving 2.10 g poloxamer 338 (BASF), 2.63 g sodium CMC (Ashland), and 18.38 g mannitol (Roquette Freres) in 369.6 g WFI and filtering the solution through a 0.2 μm filter. The formulation vehicle was added to 210 g Cabotegravir (free acid) to prepare a 400 mg/mL coarse suspension. The suspension was covered and stirred for 2 hrs. The suspension was filled into Type I glass vials and lyophilized as described in Examples 1-3. The lyophilized suspension was reconstituted to a cabotegravir concentration of 400 mg/mL.

TABLE 11
Batch #3 (400 mg/mL cabotegravir; 0.4 w/v
% P338; 0.5 w/v % NaCMC; 3.5 w/v % mannitol)

| | | Particle Size by | | |
|---|---|---|---|---|
| | | Laser Diffraction | | |
| Storage | Time | (microns) | | |
| Condition | (Months) | X10 | X50 | X90 |
| Initial | 0 | 3.4 | 7.8 | 15.2 |
| 30° C./75% | 1 | 3.3 | 7.6 | 14.6 |
| RH Upright | 3 | 3.3 | 7.7 | 15.3 |
| | 6 | 3.2 | 7.3 | 13.4 |
| | 9 | 3.3 | 7.8 | 15.3 |
| | 12 | 3.3 | 7.5 | 13.9 |
| 40° C./75% | 1 | 3.5 | 7.8 | 14.8 |
| RH Upright | 3 | 3.2 | 7.7 | 15.4 |
| | 6 | 3.2 | 7.6 | 15.6 |
| 50° C./AmbH | DY14 | 3.3 | 7.7 | 14.8 |
| Upright | 1 | 3.3 | 7.6 | 14.3 |
| | 3 | 3.3 | 7.5 | 14.5 |

Notes:
AmbH Ambient Humidity
RH Relative Humidity
— Denotes testing not scheduled at these timepoints
NGT = Not greater than
NLT = Not less than

### Example 7: Stability of a Composition Comprising Cabotegravir, sodium carboxymethylcellulose, polysorbate 80 and mannitol

A formulation vehicle was prepared by dissolving 1.04 g PS80 (Croda), 1.30 g NaCMC (Ashland; 7LF), and 9.1 g mannitol (Roquette Freres) in 183.4 g WFI. 195.0 ml of the formulation vehicle was added to 104.0 g Cabotegravir (free acid) to prepare a 400 mg/mL coarse suspension. The suspension was covered and stirred for 2 hrs. The suspension was filled into Type I glass vials and lyophilized as described in Examples 1-3. The lyophilized suspension was reconstituted to a cabotegravir concentration of 533 mg/mL.

TABLE 12
Batch #1 (533 mg/mL cabotegravir; 0.53 w/v
% PS80; 0.67 w/v % NaCMC; 4.66 w/v % mannitol)

| Storage | Time | Particle Size by Laser Diffraction (microns) | | |
|---|---|---|---|---|
| Condition | (Months) | X10 | X50 | X90 |
| Initial | 0 | 1.9 | 5.0 | 11.8 |
| 50° C./AmbH | 1 | 1.9 | 4.9 | 11.7 |
| Upright | | | | |

Notes:
AmbH Ambient Humidity
RH Relative Humidity
— Denotes testing not scheduled at these timepoints
NGT = Not greater than
NLT = Not less than

### Example 8: In Vivo Rat Pharmacokinetic Study for Compositions Comprising Cabotegravir, sodium carboxymethylcellulose, mannitol and either polysorbate 80 or poloxamer 338

Composition preparation was described in Examples 4 and 6. Formulations were dosed in 9 Sprague Dawley male rats at 30 mg/kg in the subcutaneous space of the intrascapular region. Briefly, two nanosuspension formulations of Cabotegravir at 200 (group 1) and 400 mg/ml (group 2), also differing in excipient composition, were administered subcutaneously through a single injection at target dose of 30 mg/kg in each rat. Similarly, two lyophilized powder formulations of Cabotegravir at micron-size, differing in excipient composition, were reconstituted with WFI to 400 mg/mL concentration and administered subcutaneously through a single injection at target dose of 30 mg/kg in each rat. Details of the formulations are described in Table 13.

TABLE 13
Suspension formulations details for test articles dosed in animal PK study

| | | Particle size | | | |
|---|---|---|---|---|---|
| | Cabotegravir | distribution | | | |
| | Concentration | (μm) | | | |
| Group | (mg/mL) | ×10 | ×50 | ×90 | Formulation composition |
| 1 | 200.6 | 0.10 | 0.23 | 0.60 | 2 w/v % (20 mg/mL) Polysorbate |
| | | | | | 20, 2 w/V % (20 mg/mL) |
| | | | | | Polyethylene Glycol 3350, 3.5 |
| | | | | | w/v % (35 mg/mL) Mannitol |
| 2 | 399.6 | 0.06 | 0.15 | 0.56 | 3.8 w/V % (38 mg/mL) Poloxamer |
| | | | | | 338, 3.5 w/V % (35 mg/mL) |
| | | | | | Polyethylene Glycol 3350 and 2 |
| | | | | | w/V % (20 mg/mL) Mannitol |
| 3 | 400 | 3.1 | 7.6 | 15.6 | 0.4 w/V % (4 mg/mL) Polysorbate |
| | | | | | 80, 0.5 w/V % (5 mg/mL) Sodium |
| | | | | | Carboxymethylcellulose and 3.5 (3 |
| | | | | | mg/mL) w/V % Mannitol |
| 4 | 400 | 3.0 | 7.4 | 14.9 | 0.4 w/V % (4 mg/mL) Poloxamer |
| | | | | | 338, 0.5 w/V % (5 mg/mL) Sodium |
| | | | | | Carboxymethylcellulose and 3.5 |
| | | | | | w/V % (35 mg/mL) Mannitol |

Table 14 shows Cabotegravir plasma pharmacokinetics for a preclinical study and statistical analysis through t-test for several pharmacokinetic parameters. The lyophilized formulation from Group 3 highlights a significantly reduced Cₘₐₓ (1.8 folds) and extended t_{1/2} (1.5 folds) compared to the nanosuspension formulation from Group 1. In addition, the lyophilized formulation from Group 3 demonstrates improved t_{1/2} compared to formulation from Group 4.

TABLE 14
Cabotegravir pharmacokinetic parameters for PK study in example 8 and
statistical analysis through t-test

| | | Cₘₐₓ | Tₘₐₓ | t_{1/2} | MRT_{inf} | AUCₗₐₛₜ | AUC_{inf} | Lambda_z |
|---|---|---|---|---|---|---|---|---|
| | | (ng/ml) | (h) | (h) | (h) | (h*μg/mL) | (h*μg/mL) | (1/min) |
| Group | Mean | 55250 | 360 | 462 | 859 | 48559 | 52816 | 2.74E−05 |
| 1 | SD | 12418 | 57 | 151 | 233 | 12414 | 14441 | 9.21E−06 |
| Group | Mean | 43000 | 280 | 280 | 550 | 30193 | 31289 | 4.90E−05 |
| 2 | SD | 29000 | 120 | 140 | 200 | 20721 | 22053 | 1.80E−05 |
| Group | Mean | 31000 | 470 | 700 | 1300 | 37410 | 45784 | 1.90E−05 |
| 3 | SD | 11000 | 110 | 270 | 270 | 12207 | 14775 | 9.10E−06 |
| Group | Mean | 40000 | 450 | 420 | 820 | 37126 | 39373 | 3.20E−05 |
| 4 | SD | 16000 | 120 | 160 | 170 | 16465 | 17500 | 1.40E−05 |
| p | G3 vs | 0.069 | 0.371 | ** | *** | 0.484 | 0.207 | * |
| value | G4 | | | | | | | |
| | G3 vs | *** | * | * | ** | * | 0.169 | * |
| | G1 | | | | | | | |
| | G3 vs | 0.127 | ** | *** | **** | 0.191 | 0.0604 | *** |
| | G2 | | | | | | | |

^{#} Data from 8 animals (1 animal excluded as outlier)
p value calculated from one-tailed unpaired t-test for significance:
* = 0.01 < p < 0.05;
** = 0.001 < p < 0.01;
*** = 0.0001 < p < 0.001;
p <0.0001

Tolerability, with particular focus on injection site reaction, was assessed as part of the study and results are summarized in Table 15. Occurrence of edema and scab formation were minimal and observed in few animals for a short timeframe. Nonetheless, minor differences were recorded among the 4 groups, with Groups 3 and 4 producing a lower frequency of edema, particularly Group 3.

TABLE 15
Injection site reaction observations

| | Edema | Scab formation |
|---|---|---|
| Group | Very slight in 6/9 rats - | Slight in 2/9 rats - |
| 1 | days 4-22 | days 11-43 |
| Group | Very slight in 6/9 rats - | Slight in 2/9 rats - |
| 2 | days 3-57 | days 11-15 |
| Group | Very slight in 2/9 rats - | Slight in 2/9 rats - |
| 3 | days 6-8 | days 11-57 |
| Group | Very slight in 4/9 rats - | Slight in 3/9 rats - |
| 4 | days 4-15 | days 29-43 |

### Example 9

Example 9 evaluated the safety, tolerability and pharmacokinetics of single-dose administration of a pharmaceutical composition of the invention in 64 healthy adult participants. The lyophilized formulations of Example 9 are provided in Tables 16a and 16b below:

TABLE 16a

| Formulation Components | Cohorts C1 and C2 |
|---|---|
| Component | Quantity (mg/vial) |
| Cabotegravir | 800 |
| Mannitol | 51.8 |
| Polysorbate 80 | 5.9 |
| Sodium Carboxymethylcellulose | 7.4 |
| Water for injection* | q.s. |
| Nitrogen** | q.s. |

*Water is removed during manufacturing process.
**Nitrogen is utilized as a processing aid during vial stoppering.

TABLE 16b

| Formulation Components | Cohorts C3-C7 |
|---|---|
| Component | Quantity (mg/vial) |
| Cabotegravir | 800 |
| Mannitol | 70 |
| Polysorbate 80 | 8 |
| Sodium Carboxymethylcellulose | 10 |
| Water for injection* | q.s. |
| Nitrogen** | q.s. |

*Water is removed during manufacturing process.
**Nitrogen is utilized as a processing aid during vial stoppering.

Cabotegravir particle size by diffraction (micron) is described in Table 17 below:

TABLE 17

| Quantile | Micron (μm) |
|---|---|
| X10 | 2 |
| X50 | 6 |
| X90 | 17 |

Vials containing the formulation of Table 16a were reconstituted with 1.7 mL water to achieve suspensions having a cabotegravir concentration of 400 mg/mL. Doses of the formulation of Table 16a were administered in two cohorts: 800 mg (2 ml of 400 mg/ml suspension) via the subcutaneous (SC) abdominal route (Cohort C1; 8 participants) and via the intramuscular (IM) (gluteus medius) route (Cohort C2; 8 participants).

Vials containing the formulation of Table 16b were reconstituted with either 1.7 mL water to achieve suspensions having a cabotegravir concentration of 400 mg/ml or with 1.1 mL water to achieve suspensions having a cabotegravir concentration of 533 mg/mL. Doses of the formulation of Table 16b were administered in five cohorts: 1200 mg (3 mL of 400 mg/ml suspension) via the subcutaneous (SC) abdominal route (Cohort C3; 8 participants), 1200 mg (3 mL of 400 mg/mL suspension) via the intramuscular (IM) (gluteus medius) route (Cohort C4; 8 participants), 1600 mg (3 mL of 533 mg/mL suspension) via the intramuscular (IM) (gluteus medius) route (Cohort C5; 16 participants), 2400 mg (2 injections of 2.25 mL of 533 mg/mL suspension) via the intramuscular (IM) (gluteus medius) route (Cohort C6; 9 participants), and 3200 mg (2 injections of 3 mL of 533 mg/mL suspension) via the intramuscular (IM) (gluteus medius) route (Cohort C7; 7 participants).

### Safety and Tolerability

The safety and tolerability profile of the pharmaceutical composition of Tables 16a and 16b dosed in Example 9 was acceptable. Adverse events (AEs) occurred in 63-100% of participants (Table 18). Injection site reactions (ISRs) were the most common AEs and were mostly Grade 1 (Table 19). Overall, fewer ISRs were reported following IM administration compared to SC administration; however, ISRs were predominantly Grade 1 (Table 19). No Grade 4 or serious AEs have been reported in Cohorts C1 to C7.

TABLE 18
Summary of most frequent (≥2 participants across cohorts)
AEs (ISR and non-ISR) by cohort

| | Cohort | Cohort | Cohort | Cohort | Cohort | Cohort | Cohort |
|---|---|---|---|---|---|---|---|
| | C1 | C3 | C3 | C4 | C5 | C6 | C7 |
| | 800 mg | 800 | 1200 | 1200 | 1600 | 2400 | 3200 |
| Most Frequent | SC | mg IM | mg SC | mg IM | mg IM | mg IM | mg IM |
| Adverse Events | (N = 8) | (N = 8) | (N = 8) | (N = 8) | (N = 16) | (N = 9) | (N = 7) |
| Any event, n (%) | 8 (100) | 5 (63) | 8 (100) | 8 (100) | 12 (75) | 8 (100) | 7 (100) |
| Any ISR, n (%) | 8 (100) | 3 (38) | 8 (100) | 8 (100) | 11 (69) | 8 (89) | 7 (100) |
| ISRs | | | | | | | |
| Injection site erythema | 8 (100) | 0 | 7 (88) | 0 | 1 (6) | 1 (11) | 0 |
| Injection site pain | 3 (38) | 2 (25) | 5 (63) | 8 (100) | 8 (50) | 7 (78) | 7 (100) |
| Injection site nodule | 6 (75) | 0 | 8 (100) | 0 | 2 (13) | 5 (56) | 1 (14) |
| Injection site swelling | 3 (38) | 2 (25) | 2 (25) | 0 | 3 (19) | 2 (22) | 1 (14) |
| Injection site pruritus | 0 | 0 | 1 (13) | 0 | 0 | 2 (22) | 1 (14) |
| Non-ISRS | | | | | | | |
| Nasopharyngitis/ | 1 (13) | 0 | 0 | 0 | 1 (6) | 0 | 0 |
| pharyngitis | | | | | | | |
| streptococcal | | | | | | | |
| (Viral) upper | 1 (13) | 0 | 2 (25) | 0 | 0 | 0 | 0 |
| respiratory tract | | | | | | | |
| infection | | | | | | | |
| Urinary tract infection | 0 | 0 | 1 (13) | 0 | 0 | 1 (11) | 0 |
| Abdominal | 0 | 2 (25) | 1 (13) | 0 | 1 (6) | 0 | 0 |
| discomfort/abdominal | | | | | | | |
| pain lower/abdominal | | | | | | | |
| pain upper | | | | | | | |
| Diarrhoea | 0 | 1 (13) | 0 | 0 | 1 (6) | 1 (11) | 0 |
| Nausea | 0 | 0 | 0 | 0 | 1 (6) | 0 | 1 (14) |
| Dizziness | 0 | 0 | 1 (13) | 1 (13) | 0 | 0 | 1 (14) |
| Headache | 0 | 1 (13) | 1 (13) | 2 (25) | 0 | 1 (11) | 1 (14) |
| Back pain | 0 | 0 | 0 | 0 | 2 (13) | 0 | 0 |
| Cough | 0 | 1 (13) | 0 | 0 | 1 (6) | 0 | 1 (14) |
| Pollakiuria | 0 | 0 | 0 | 0 | 2 (13) | 0 | 0 |
| Blood creatine | 0 | 0 | 0 | 1 (13) | 0 | 1 (11) | 0 |
| phosphokinase | | | | | | | |
| increased | | | | | | | |

TABLE 19
Summary of ISRs

| | Cohort C1 | Cohort C2 | Cohort C3 | Cohort C4 | Cohort C5 | Cohort C6 | Cohort C7 |
|---|---|---|---|---|---|---|---|
| | SC | IM | SC | IM | IM | IM | IM |
| | 800 mg | 800 mg | 1200 mg | 1200 mg | 1600 mg | 2400 mg | 3200 mg |
| PARAMETER, N | [2 mL] | [2 mL] | [3 mL] | [3 mL] | [3 mL] | [4.5 mL] | [8 mL] |
| (% OF PARTICIPANTS) | (n = 8) | (n = 8) | (n = 8) | (n = 8) | (n = 16) | (n = 9) | (n = 7) |
| | Single injection | | | | | Two injections | |
| Any ISR | 8 (100) | 3 (38) | 8 (100) | 8 (100) | 11 (69) | 8 (89) | 7 (100) |
| Drug-related | 8 (100) | 3 (38) | 8 (100) | 8 (100) | 10 (83) | 8 (69) | 7 (100) |
| Serious | 0 | 0 | 0 | 0 | 0 | 0 | 0 |
| Grade 3 | 0 | 0 | 0 | 0 | 0 | 1 | 0 |
| Grade 4 | 0 | 0 | 0 | 0 | 0 | 0 | 0 |
| Pain | 3 (38) | 2 (25) | 5 (63) | 8 (100) | 8 (50) | 7 (78) | 7 (100) |
| Grade 1 | 3 (30) | 1 (13) | 5 (83) | 8 (100) | 8 (50) | 7 (78) | 5 (71) |
| Grade 2 | 0 | 1 (13) | 0 | 0 | 0 | 0 | 2 (29) |
| Grade 3 | 0 | 0 | 0 | 0 | 0 | 0 | 0 |
| Erythema | 8 (100) | 0 | 7 (88) | 0 | 1 (6) | 1 (11) | 0 |
| Grade 1 | 6 (75) | 0 | 5 (63) | 0 | 1 (8) | 0 | 0 |
| Grade 2 | 2 (29) | 0 | 2 (25) | 0 | 0 | 0 | 0 |
| Grade 3 | 0 | 0 | 0 | 0 | 0 | 1 (11) | 0 |
| Swelling/induration | 3 (38) | 2 (25) | 2 (26) | 0 | 3 (10) | 2 (22) | 1 (14) |
| Grade 1 | 3 (38) | 2 (25) | 2 (25) | 0 | 2 (13) | 1 (11) | 1 (14) |
| Grade 2 | 0 | 0 | 0 | 0 | 1 (6) | 1 (11) | 0 |
| Grade 3 | 0 | | | | | 0 | 0 |
| Nochnie | 6 (9) | 0 | 8 (100) | 0 | 2 (13) | $ (58) | 1 (14) |
| Grade 1 | 0 | 0 | 8 (100) | 0 | 2 (13) | 5 (66) | 1 (14) |
| Grade 2 | 0 | 0 | 0 | 0 | 0 | 0 | 0 |
| Grade 3 | 0 | 0 | 0 | 0 | 0 | 0 | 0 |
| Total ISRs (% of total ISRs) | 21 | 8 | 24 | 9 | 15 | 33 | 21 |
| Grade 1 | 19 (80) | 4 (80) | 22 (92) | 9 (100) | 14 (93) | 29 (86) | 17 (81) |
| Grade 2 | 2 (10) | 1 (20) | 2 (8) | 0 | 1 (7) | 3 (9) | 4 (19) |
| Grade 3 | 0 | | | | | 1 (3) | 0 |
| Grade 4 | 0 | | | | | 0 | |
| Median ISR duration (IQR) | | | | | | | |
| days [n = events with | | | | | | | |
| non-missing end date/total | | | | | | | |
| number of events] ^{a} | | | | | | | |
| Pain | n = 3/3 | n = 2/2 | n = 5/6 | n = 8/8 | n = 8/8 | n = 13/13 | A = 14/14 |
| | 7 | 6.5 | 8 | 6 | 6 | 7 | 6.5 |
| | (6-12) | (5-8) | (6-15) | (3.5-6.5) | (4-7) | (6-10) | (4-10) |
| Erythema | n = 8/8 | N/A | n = 7/7 | N/A | n = 1/1 | n = 2/2 | N/A |
| | 16 | | 15 | | 4 | 5 | |
| | (10-41) | | (13-31) | | (4-4) | (5-5) | |
| Swelling/induration | n = 3/3 | n = 3/3 | n = 2/2 | N/A | n = 3/3 | n = 4/4 | n = 2/2 |
| | 42 | 5 | 1.5 | | 5 | 38.5 | 2 |
| | (13-63) | (4-43) | (1-2) | | (5-19) | (5-72) | (2-2) |
| Nodule | n = 0/6 | N/A | n = 1/8 | N/A | n = 1/2 | n = 7/9 | n = 1/1 |
| | | | 106 | | 43 | 55 | 10 |
| | | | (106-108) | | (43-43) | (19-106) | (10-10) |

^{a} ISR duration is not calculated if end date is missin

### Example 10. PK and Simulations

Cabotegravir plasma Cmax following a single SC (abdominal) or IM (gluteal) injection of the pharmaceutical compositions described in Example 9 (Cohorts C1-C7) was lower than that following a single IM injection of APRETUDE®, while plasma Cmax following bilateral 2.25 mL IM gluteal injections (4.5 mL total) was comparable to that following a single IM injection of APRETUDE®.

Based on observed PK data for cohorts C1 to C7 in Example 9, t1/2 following these cohorts SC and IM injection is predicted to be >6x (SC injections) and >2x (IM injections) the t1/2 of APRETUDE® (Q2M cabotegravir 200 mg/mL), respectively.

TABLE 20
Plasma Pharmacokinetic and Safety Results in Healthy Adult Participants
(Cohorts C1-C7)

| | | | Cohort | Cohort | Cohort | Cohort | Cohort |
|---|---|---|---|---|---|---|---|
| | | | C3 | C4 | C5 | C6 | C7 |
| | Cohort C1 | Cohort C2 | (3 mL) | (3 mL) | (3 mL) | (2 × | (2 × 3 |
| | (2 mL) SC | (2 mL) IM | SC | IM | IM | 2.25 mL) | mL) IM |
| Parameter | (N = 8) | (N = 8) | (N = 8) | (N = 8) | (N = 16) | IM (N = 9) | (N = 7) |
| Cmax, μg/mL^{a} | 0.7 (35.1) | 1.8 (43.0) | 0.9 (32.6) | 1.8 (130.7) | 1.9 (85.3) | 4.1 (40.4) | 6.5 (61) |
| Grade 1 ISRs, | 6 (75) | 2 (25) | 6 (75) | 8 (100) | 10 (63) | 7 (78) | 5 (71) |
| n (%) | | | | | | | |
| Grade 2 ISRs, | 2 (25) | 1 (13) | 2 (25) | 0 | 1 (6) | 0 | 2 (29) |
| n (%) | | | | | | | |
| Grade ≥3 | 0 | 0 | 0 | 0 | 0 | 1 (11) | 0 |
| ISRs, n (%) | | | | | | | |

ªValue reported as geometric mean (% CVb).

### Simulations

In a first set of simulations, a cabotegravir population pharmacokinetic (PopPK) model was built based on PK data following cabotegravir (CAB) 200 mg/mL intramuscular (IM) injections collected in 16 historical studies (Population pharmacokinetics of cabotegravir following administration of oral tablet and long-acting intramuscular injection in adult HIV-1-infected and uninfected subjects. Han K, Baker M, Lovern M, Paul P, Xiong Y, Patel P, Moore K P, Seal C S, Cutrell A G, D'Amico R D, Benn P D, Landovitz R J, Marzinke M A, Spreen W R, Ford S L. Br J Clin Pharmacol. 2022 October;88 (10): 4607-4622. doi: 10.1111/bcp. 15439. Epub 2022 Jul. 4. PMID: 35695476) and updated based on PK data from 19 studies: LAI116585, LAI117010, LAI117011, LAI117020, 201741, 201479, 201480, 205696, LAI116482 (LATTE), LAI115428, LAI116815, 200056 (LATTE-2), 201120 (ECLAIR), 201103 (HPTN077), 201584 (FLAIR), 201585 (ATLAS), 207966 (ATLAS-2M), 201738 (HPTN 083) and 201739 (HPTN 084). A total of 34,850 CAB plasma concentrations collected at various time points from 2,694 participants were used to build this PopPK model. It was discovered that:
Long-acting intramuscular gluteal absorption rate constant (KA2) was 46.5% lower in people assigned female at birth than people assigned male at birth.
KA2 was 50.8% higher if the injection was split in people assigned male at birth, but KA2 was not associated with split injections in people assigned female at birth.
KA2 decreased with increasing BMI, and decreased more in people assigned male at birth than people assigned female at birth for the same amount of BMI increase.
KA2 was lower with longer needle length.
KA2 was 22.8% higher in study 201120 (ECLAIR) than other studies after adjusting for all covariates.
Apparent central clearance (CL/F) was 16.4% higher in current smokers.

Based on the observed PK data from the study discussed in Example 9, this PopPK model was modified by setting the typical value of half-life (t ½) to 25 weeks for 533 mg/ml formulations and twice that if APRETUDE® for 400 mg/mL formulations to perform simulations for the cabotegravir formulations used in Example 9. It was assumed that:
t ½ following IM gluteal injections of the formulations in Example 9 with a concentration of 533 mg/ml of cabotegravir is 25 weeks,
t ½ following IM gluteal injections of the formulations in Example 9 for formulations with a concentration of 400 mg/mL is twice the t ½ of APRETUDE®,
effects of body weight, BMI and smoking status on PK, all variabilities (e.g., inter-individual variability and residual variability), and bioavailability are the same as for the 200 mg/mL IM gluteal injections,
PK following split IM gluteal injections of the formulations in Example 9 on both sides of the body is identical to PK following a single IM gluteal injection of the formulations in Example 9.

The simulations were performed as follows: covariates including body weight, BMI and smoking status were resampled 5000 times from the distribution of these covariates for males and females, generating 5000 virtual male subjects and 5000 virtual female subjects. Individual P K parameters of the virtual subjects were calculated using subject-specific covariates and using the population parameter estimates, subject-specific NONMEM inter-individual errors (ETAs) sampled from the distributions that are decided by the estimated variance-covariate matrix of between-subject variability from the final population PK model. Concentration-versus-time profiles of the virtual subjects were calculated using the individual PK parameters. Residual variability (EPS) was included in the simulation. Median and 10th and 90th percentiles (80% prediction interval) of the simulated concentration-versus-time profiles of the virtual males and females were calculated.

An adequate dose was defined as the dose that would meet these criteria:
1. maintain the median and 10th percentile of trough CAB plasma concentrations (Ctau) higher than those from the approved Q2M regimen of 200 mg/mL CAB IM gluteal injections in both people assigned male at birth and people assigned female at birth or
2. maintain Ctau higher than the sex-specific PrEP benchmarks in more than 90% of participants. The sex-specific PrEP benchmarks are the 10th percentile of Ctau observed in the Phase 3 studies 201738 (HPTN 083) for people assigned male at birth (1.05 μg/mL) and 201739 (HPTN 084) for people assigned female at birth (1.39 μg/mL).

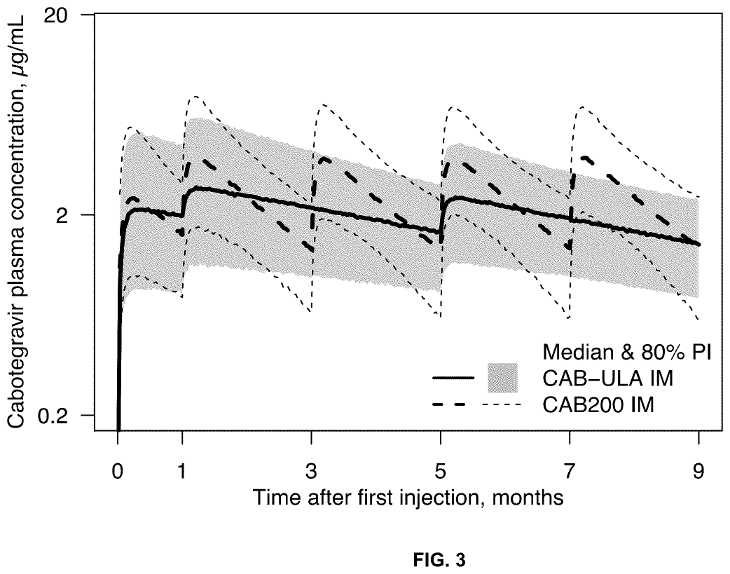

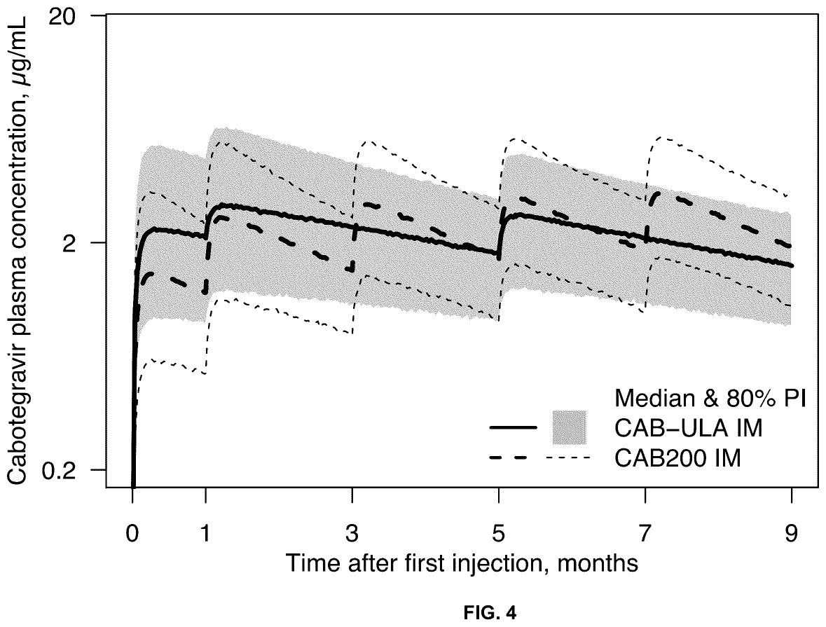

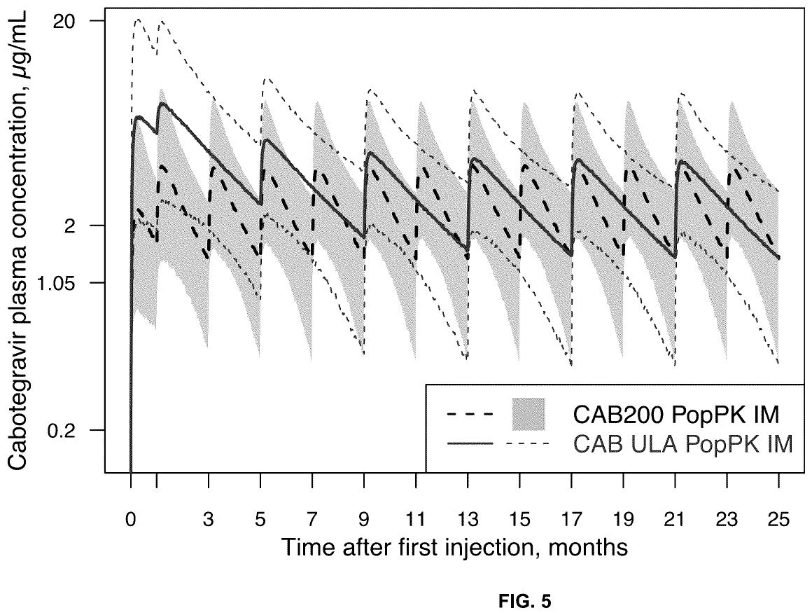

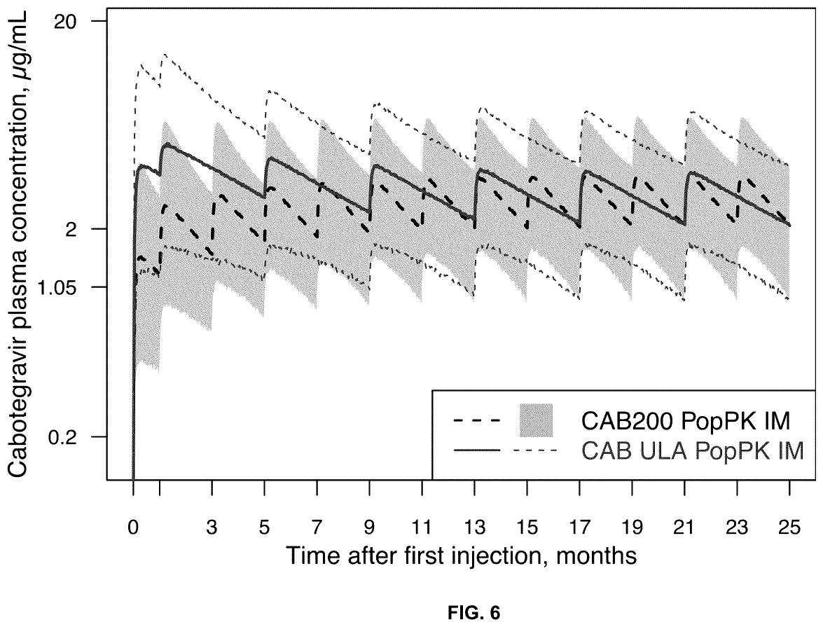

The simulations show that a Q4M regimen of IM gluteal injections with a maintenance dose of 1.5 mL of 533 mg/ml of cabotegravir (799.5 mg cabotegravir) (FIGS. 3 and 4) or 3 mL of 400 mg/mL cabotegravir (1200 mg cabotegravir) (FIGS. 5 and 6) would not maintain CAB plasma concentrations higher than the approved Q2M regimen of CAB 200 mg/mL IM gluteal injections in people assigned male or female at birth.

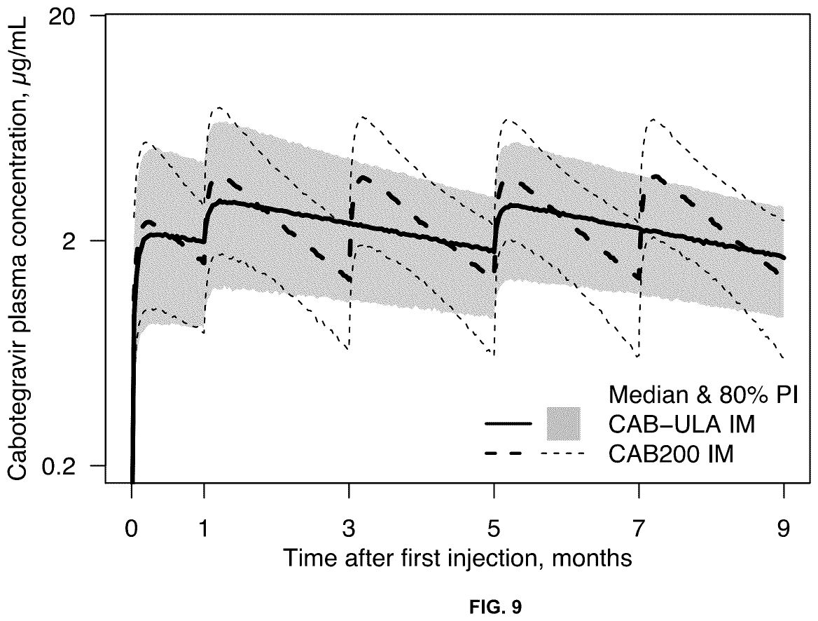

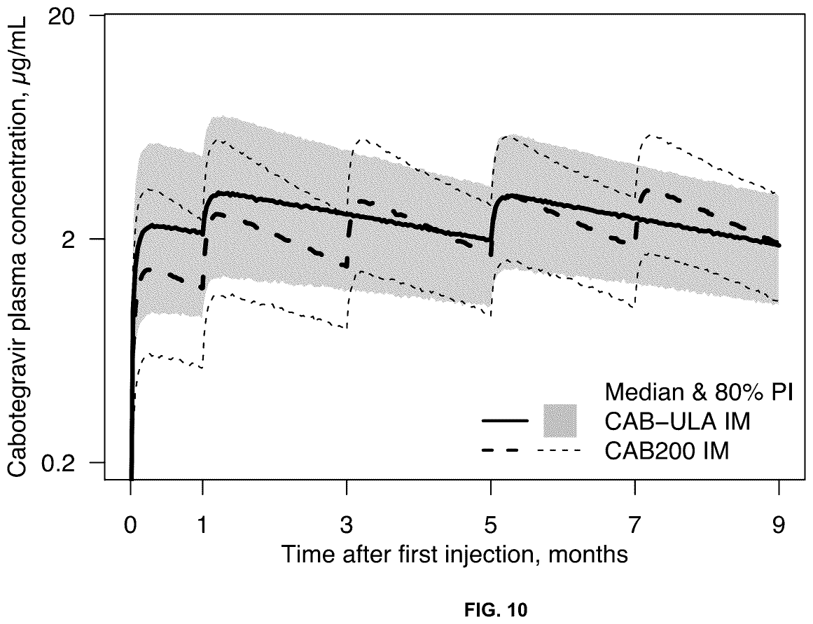

A Q4M regimen of IM gluteal injections with a loading dose of at least 2132 mg (4 mL) and a maintenance dose of at least 1066 mg (2 mL) administered Q4M starting 1 month after the loading dose would meet criterion 1 i.e. would maintain CAB plasma concentrations higher than the approved Q2M regimen of CAB200 IM gluteal injections in both people assigned male at birth (FIG. 9) and people assigned female at birth (FIG. 10).

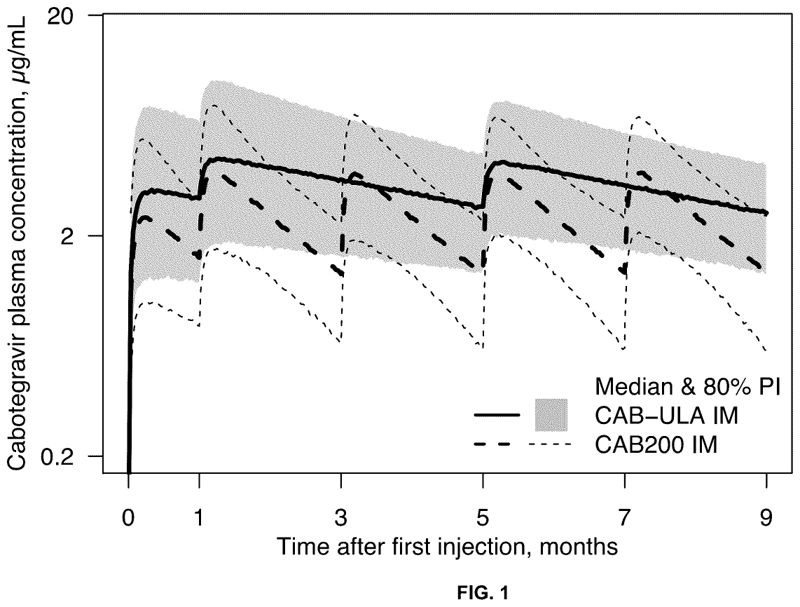

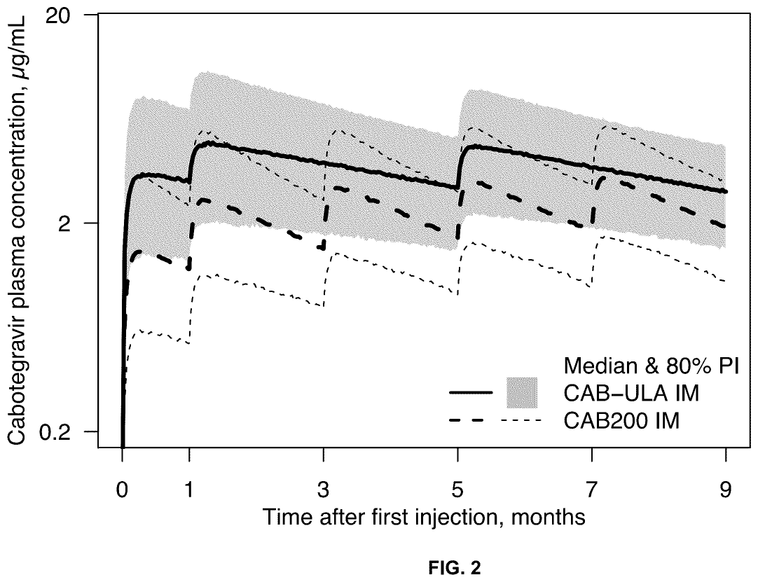

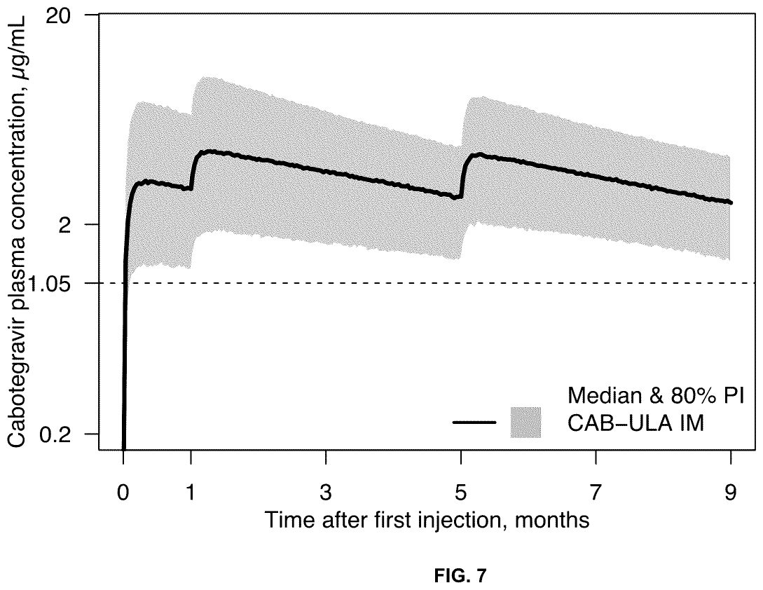

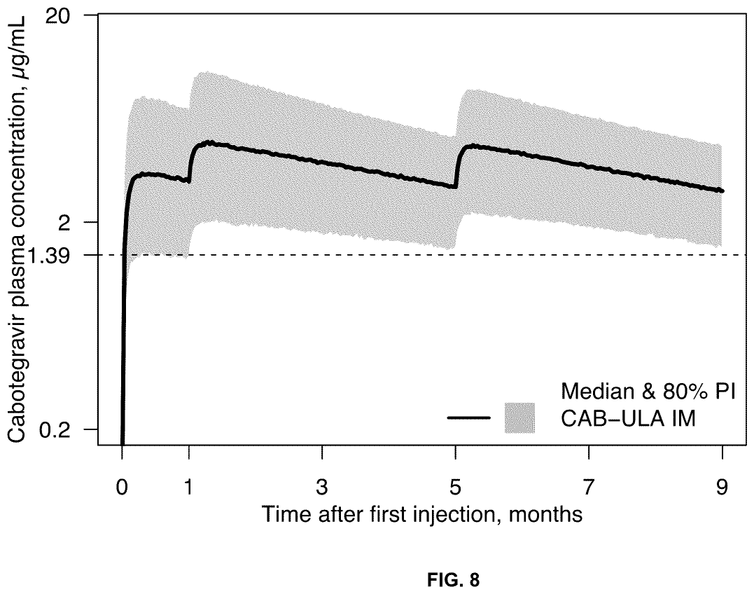

A Q4M regimen of IM gluteal injections with a loading dose of at least 3200 mg (3 mL+3 mL) and a maintenance dose of at least 1600 mg (3 mL) that is administered Q4M starting at 1 month after the loading dose would meet both criteria:
maintain CAB plasma concentrations higher than the approved Q2M regimen of CAB200 IM gluteal injections in both people assigned male at birth (FIG. 1) and people assigned female at birth (FIG. 2).
maintain Ctau higher than 1.05 μg/mL and 1.39 μg/mL in more than 90% of people assigned male at birth (FIG. 7) and people assigned female at birth (FIG. 8), respectively. As shown in FIGS. 7 and 8, the lower boundary of the grey band (simulated 10th percentile of CAB-ULA) is higher than the horizontal dashed line (benchmark).

In a second set of simulations, a cabotegravir population pharmacokinetic (PopPK) model for CAB LA (APRETUDER) was built and updated based on PK data following CAB 200 mg/mL IM injections collected in 19 historical studies. The CAB LA model was used as a starting point for CAB ULA (Cab 533 mg/mL Q4M) model development. The systemic cabotegravir parameters, which are independent of formulation (clearance and volume), and their inter-individual variabilities (IIV), were fixed to the CAB LA PopPK model parameter estimates. Parameters with the potential to be influenced by formulation, such as absorption parameters and their IIVs, were re-estimated based on CAB ULA plasma concentration data from the study shown in Example 9 following the administration of single doses of CAB ULA via the intramuscular (gluteus medius) route, from 800 to 3200 mg, in a total of 48 healthy participants.

The CAB ULA PopPK model was used to simulate concentration-time profiles for virtual subjects as follows: covariates including body weight, BMI and age were resampled 5000 times from the distribution of these covariates for males and females, generating 5000 virtual male subjects and 5000 virtual female subjects. Individual P K parameters of the virtual subjects were calculated using subject-specific covariates and using the population parameter estimates, subject-specific NONMEM inter-individual errors (ETAs) sampled from the distributions that are decided by the estimated variance-covariate matrix of between-subject variability from the final population PK model. Concentration-versus-time profiles of the virtual subjects were calculated using the individual PK parameters. Residual variability (EPS) was included in the simulation. Median and corresponding 80% and/or 90% prediction intervals of the simulated concentration-versus-time profiles of the virtual males and females were calculated, as was the percentage of trough concentrations above (1) the PrEP PK benchmark of 1.39 μg/mL (Study HPTN 084) for females, the PrEP PK benchmark of 1.05 μg/mL (Study HPTN 083) for males, and the Phase 3 benchmark for treatment in both males and females of 0.45 μg/mL (Studies 201584 [FLAIR], 201585 [ATLAS] and 207966 [ATLAS-2M]).

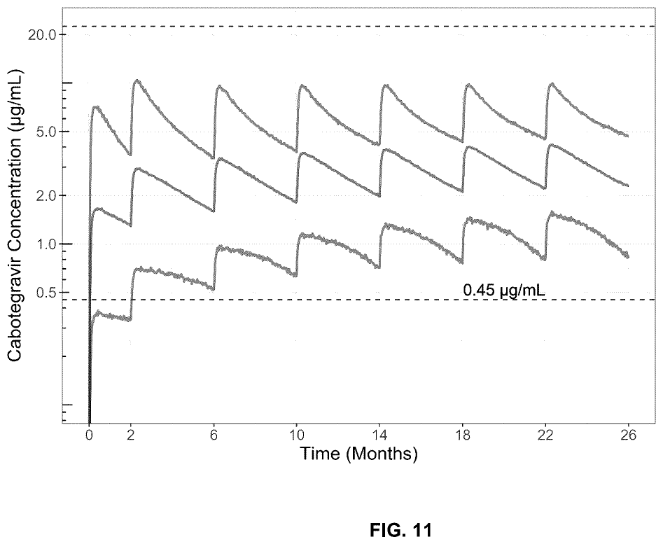

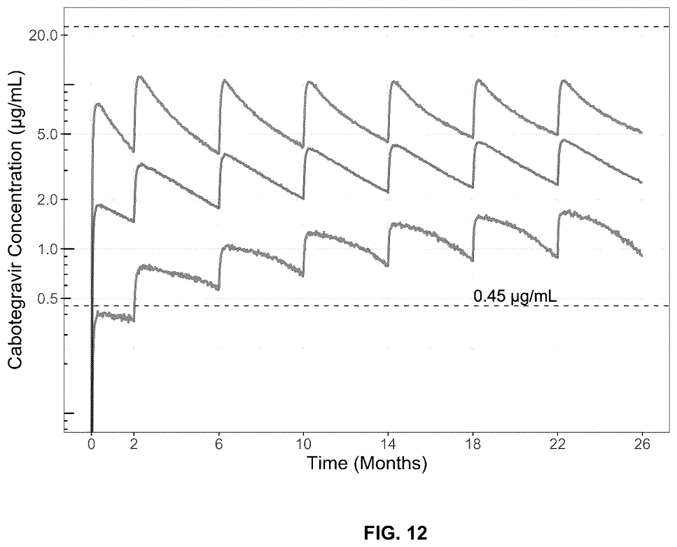

Updated simulations from the second set of simulations based on the CAB ULA PopPK model suggest that a 1600 mg CAB ULA (3 mL of 533 mg/mL cabotegravir) IM gluteal injection (loading dose) followed by a maintenance regimen of 1600 mg CAB ULA (3 mL of 533 mg/mL cabotegravir) IM gluteal injections administered Q4M starting 2 months after the loading dose would maintain CAB plasma concentrations higher than the approved Q2M regimen of CAB200 IM gluteal injections in both people assigned male at birth (FIG. 11) and people assigned female at birth (FIG. 12), and above relevant PK benchmarks for treatment and pre-exposure prophylaxis.

It will be understood that the present invention has been described purely by way of example, and modification of detail can be made within the scope of the invention. Each feature disclosed in the description, and where appropriate the claims and drawings, may be provided independently or in any appropriate combination.

1. A method of preventing HIV in a human, comprising administering to said human a pharmaceutical composition comprising cabotegravir once every 15 to 24 weeks.

2. A method of treating HIV in a human in need thereof, comprising administering to said human a pharmaceutical composition comprising cabotegravir once every 15 to 24 weeks.

3. The method according to claim 1, wherein the cabotegravir provides Ctau of at least 1 μg/mL.

4. The method according to claim 3, wherein the human was assigned female at birth and the cabotegravir provides a Ctau of at least about 1.39 μg/mL; or wherein the human was assigned male at birth and the cabotegravir provides a Ctau of at least about 1.05 μg/mL.

5. The method according to claim 1, wherein the cabotegravir provides a Ctau higher than the Ctau achieved in the dosing regimen of APRETUDE®.

6. The method according to claim 1, wherein the method comprises:
a. first administering a loading dose of the pharmaceutical composition to the human;
b. 2 to 6 weeks after administering the loading dose, administering a maintenance dose of the pharmaceutical composition to the human; and then
c. administering the maintenance dose every 15 to 24 weeks thereafter.

7. The method according to claim 6, wherein the loading dose of the pharmaceutical composition comprises about 600 mg to about 4000 mg of cabotegravir.

8. The method according to claim 6, wherein the maintenance dose of the pharmaceutical composition comprises about 1300 to about 1700 mg of cabotegravir.

9. The method according to claim 1, wherein the pharmaceutical composition further comprises polysorbate 80, sodium carboxymethylcellulose, and mannitol.

10. The method according to claim 1, wherein the cabotegravir in the pharmaceutical composition is present in the form of particles with an X50 value of between (and including) 2.5 μm and 10 μm.

11. The method according to claim 1, wherein a concentration of cabotegravir in the pharmaceutical composition is in a range of from about 300 mg/mL to about 650 mg/mL.

12. The method according to claim 1, wherein the pharmaceutical composition has been reconstituted from a lyophilized powder with a suitable liquid.

13.-20. (canceled)

21. The method according to claim 6, wherein the maintenance dose is administered once every 15 to 20 weeks.

22.-27. (canceled)

28. The method according to claim 2, wherein the cabotegravir provides Ctau of at least 1 μg/mL.

29. The method according to claim 28, wherein the human was assigned female at birth and the cabotegravir provides a Ctau of at least about 1.39 μg/mL; or wherein the human was assigned male at birth and the cabotegravir provides a Ctau of at least about 1.05 μg/mL.

30. The method according to claim 2, wherein the cabotegravir provides a Ctau higher than the Ctau achieved in the dosing regimen of APRETUDE®.

31. The method according to claim 2, wherein the method comprises:
a. first administering a loading dose of the pharmaceutical composition to the human;
b. 2 to 6 weeks after administering the loading dose, administering a maintenance dose of the pharmaceutical composition to the human; and then
c. administering the maintenance dose every 15 to 24 weeks thereafter.

32. The method according to claim 31, wherein the loading dose of the pharmaceutical composition comprises about 600 mg to about 4000 mg of cabotegravir.

33. The method according to claim 31, wherein the maintenance dose of the pharmaceutical composition comprises about 1300 to about 1700 mg of cabotegravir.

34. The method according to claim 2, wherein the pharmaceutical composition further comprises polysorbate 80, sodium carboxymethylcellulose, and mannitol.

35. The method according to claim 2, wherein the cabotegravir in the pharmaceutical composition is present in the form of particles with an X50 value of between (and including) 2.5 μm and 10 μm.

36. The method according to claim 2, wherein a concentration of cabotegravir in the pharmaceutical composition is in a range of from about 300 mg/mL to about 650 mg/mL.

37. The method according to claim 2, wherein the pharmaceutical composition has been reconstituted from a lyophilized powder with a suitable liquid.

38. The method according to claim 31, wherein the maintenance dose is administered once every 15 to 20 weeks.
